(19) United States
(12) Patent Application Publication
(10) Pub. No.: US 2016/0221965 A1
(43) Pub. Date: Aug. 4, 2016

(54) DISUBSTITUTED TRIFLUOROMETHYL PYRIMIDINONES AND THEIR USE

(71) Applicant: BAYER PHARMA AKTIENGESELLSCHAFT, Berlin (DE)

(72) Inventors: Alexander STRAUB, Wuppertal (DE); Marie-Pierre COLLIN, Wuppertal (DE); Michael KOCH, Schwelm (DE); Jutta MEYER, Solingen (DE); Karl-Heinz SCHLEMMER, Wuppertal (DE); Carl Friedrich NISING, Berlin (DE); Nicole BIBER, Wuppertal (DE); Sonja ANLAUF, Wermelskirchen (DE); Matthias Beat WITTWER, Wuppertal (DE)

(73) Assignee: Bayer Pharma Aktiengesellschaft, Berlin (DE)

(21) Appl. No.: 15/021,372

(22) PCT Filed: Sep. 12, 2014
(86) PCT No.: PCT/EP2014/069540

§ 371 Date: Mar. 11, 2016

(30) Foreign Application Priority Data
Sep. 16, 2013 (EP) 13184481.3

(51) Int. Cl. C07D 239/47; A61K 45/06; C07D 401/12; C07D 239/56; C07D 239/36; C07D 401/06; A61K 31/513; C07D 239/52

(52) U.S. Cl. CPC C07D 239/47; A61K 31/513; A61K 45/06; C07D 239/52; C07D 239/56; C07D 239/36; C07D 401/06; C07D 401/12

(57) ABSTRACT

The present application relates to novel 2,5-disubstituted 6-(trifluoromethyl)pyrimidin-4(3H)-one derivatives, to processes for their preparation, to their use alone or in combinations for the treatment and/or prevention of diseases, and to their use for preparing medicaments for the treatment and/or prevention of diseases, in particular for treatment and/or prevention of cardiovascular, renal, inflammatory and fibrotic diseases.

The present application relates to novel 2,5-disubstituted 6-(trifluoromethyl)pyrimidin-4(3H)-one derivatives, to processes for their preparation, to their use alone or in combinations for the treatment and/or prevention of diseases, and to their use for preparing medicaments for the treatment and/or prevention of diseases, in particular for treatment and/or prevention of cardiovascular, renal, inflammatory and fibrotic diseases.

### BACKGROUND OF THE INVENTION

Chemotactic cytokines or chemokines can be produced in most tissues, such as heart, kidney and lung, but also vessels, in the context of the immune response to tissue injury or inflammatory stimuli, for example bacterial toxins. They are essential for the recruitment of specific leukocyte subpopulations (such as neutrophiles, monocytes, basophiles, eosinophiles, effector-T-cells, dendritic cells) to the site of an inflammation [Mackay, Nature Immunol. 2 (2), 95-101 (2001)]. Binding to glycosaminoglycans of the extracellular matrix and the endothelium results in a local chemokine concentration gradient which allows chemotactic leukocyte migration to the inflammation or infection site in the body [Tanaka et al., Nature 361, 79-82 (1993); Luster, N. Engl. 0.1 Med. 338 (7), 436-445 (1998)]. By virtue of the recruitment of inflammatory cells, chemokines therefore play a central role in the genesis and progression of numerous inflammatory disorders [Schall, Cytokine 3, 165-183 (1991); Schall et al., Curr. Opin. Immunol. 6, 865-873 (1994)]. In addition to the chemotactic action chemokines are also involved in the regulation of haematopoiesis, cell proliferation, angiogenesis or tumour growth, inter alia.

According to organization and position of conserved cysteine residues, the chemokines are classified into four different sub-groups (CXC, CC, C and CX3C) [Bacon et al., J. Interferon Cytokine Res. 22 (10), 1067-1068 (2002)]. The largest family are the CC chemokines, which also include the classic inflammatory chemokines such as the MCPs (monocyte chemoattractant proteins) whose expression is induced in most tissues in the case of tissue damage or infection via proinflammatory cytokines such as IL-1, TNF-α or IFN-γ [Rollins, in: Cytokine Reference, Oppenheim et al., Ed., Academic Press, London, 1145-1160 (2000)]. The 48 chemokines hitherto identified in man bind to specific chemokine receptors which belong to the family of the G-protein-coupled receptors.

The CC chemokine receptor CCR2 is expressed inter alia on the surface of macrophages, monocytes, B cells, activated T cells, dendritic cells, epithelial cells and activated endothelial cells and binds the inflammatory chemokines MCP-1 (CCL2), MCP-2 (CCL8), MCP-3 (CCL7) and MCP-4 (CCL13). As the only ligand, MCP-1 appears to bind selectively to CCR2 [Struthers and Pasternak, Current Topics in Medicinal Chemistry 10 (13), 1278-1298 (2010)]. MCP-1 is expressed inter alia by cardiomyocytes, mesangial cells, alveolar cells, T lymphocytes, macrophages and monocytes [Deshmane et al., J. Interferon Cytokine Res. 29, 313-326 (2009)]. The CC chemokine receptor CCR2 is also the only high affinity receptor for MCP-1 characterized [Struthers and Pasternak, Current Topics in Medicinal Chemistry 10 (13), 1278-1298 (2010)]. In man, CCR2 is expressed on most blood monocytes [Tacke and Randolph, Immunobiology 211, 609-618 (2006)]. The activation of CCR2 by MCP-1 plays an important role in the infiltration and activation of monocytes [Dobaczewski and Frangogiannis, Frontiers in Bioscience S1, 391-405 (2009); Charo and Ransohoff, N Engl. J. Med. 354 (6), 610-621 (2006)] in the context of the cellular immune response and in chronic inflammatory processes, for example in the heart and the kidney. This infiltration of monocytes and their differentiation in macrophages also represents a second source of pro-inflammatory modulators such as TNF-α, IL-8, IL-12 and matrix metalloproteases (MMPs), inter alia.

Furthermore, CCR2 mediates the migration of monocytes from the bone marrow and their subsequent invasion of inflammatory regions [Carter, Expert Opin. Ther. Patents 23 (5), 549-568 (2013)]. In addition, it appears that fibrocytes may also be formed from the population of the CCR2+ monocytes [Dobaczewski and Frangogiannis, Frontiers in Bioscience S1, 391-405 (2009)], which implies a role of CCR2 in fibrosis (for example of the lung or the liver). The CCR2-mediated invasion of monocytes is also one of the first steps of the formation of atherosclerosis [Gu et al., Mol. Cell 2 (2), 275-281 (1998)].

Experiments with animal models have shown that inhibition of the interaction of MCP-1 and CCR2—by inhibiting the activation of CCR2 using specific antagonists or MCP-1-selective antibodies or by genetic deletion (knock-out) of MCP-1 or CCR2—can reduce an inflammatory response in various disorders and monocyte-infiltration into inflamed lesions can be reduced (arthritis, asthma). CCR2/MCP-1-mediated cellular responses are involved in numerous disorders such as cardiomyopathies, myocardial infarction, myocarditis, chronic heart failure, diabetic renal disease, acute kidney damage, rheumatoid arthritis, multiple sclerosis, chronic-obstructive pulmonary disease (COPD), asthma, atherosclerosis, inflammatory bowel diseases (IBD), diabetes, neuropathic pain, macular degeneration, angiogenesis and cancer [Struthers and Pasternak, Current Topics in Medicinal Chemistry 10 (13), 1278-1298 (2010); Carter, Expert Opin. Ther. Pat. 23 (5), 549-568 (2013); Higgins et al., in: Chemokine Research, Basic Research and Clinical Application, Vol. II, Birkhauser-Verlag, 115-123 (2007)].

### CCR2 and Heart Failure/Cardioprotection:

In myocardial infarction, neutrophiles accumulate in the first hours after ischaemia, with maximum accumulation after one day. Various experimental studies on animals have confirmed that subsequently, in the first two weeks after infarction, monocytes and macrophages dominate the cell infiltrate [Nahrendorf et al., Circulation 121, 2437-2445 (2010)]. This is accompanied by upregulation of MCP-1 [Hayasaki et al., Circ. J. 70 (3), 342-351 (2006)]. Neutrophiles and also monocytes and macrophages produce local proteolytic enzymes and reactive oxygen species (ROS), thus damaging the cardiomyocytes which have survived the ischaemic period. Preclinical studies have shown that the infarct size can be reduced by anti-inflammatory treatment. It is expected that such a protection will also occur in patients suffering from acute myocardial infarction, which may reduce the infarct size and prevent a worsening of the cardiac function after the infarct.

CCR2-deficient mice show a reduction of the infarct size and reduced remodelling after myocardial infarction [Hayasaki et al., Circ. J. 70 (3), 342-351 (2006)]. Likewise, MCP-1-deficient mice have reduced remodelling after myocardial infarction [Dewald et al., Circ. Res. 96 (8), 881-889 (2005)]. In particular, ApoE^{-/-} mice also show significantly improved infarct healing if the CCR2 receptor is blocked [Majmudar et al., Circulation 127, 2038-2046 (2013)]. In addition, it has been described that, compared to healthy controls, monocytes in patients suffering from heart failure release more MCP-1 [Aukrust et al., Circulation 97, 1136-1143 (1998); Aukrust et al., Arterioscler. Thromb. Vasc. Biol. 28, 1909-1919 (2008)], and increased MCP-1 plasma levels were also detected in patients with atrial fibrillation [Li et al., Heart Rhythm 7, 438-444 (2010)].

### CCR2 and Kidney Function/Nephroprotection:

Immunological and inflammatory mechanisms play a crucial role in the development and progression of diabetic nephropathy. Here, monocytes and/or macrophages have a substantial effect in the pathogenesis [Chow et al., Kidney Int. 65, 116-128 (2004); Chow et al., Kidney Int. 69, 73-80 (2006)]. Deletion of CCR2 or blocking of the MCP-1 signal path reduces macrophage infiltration and reduces kidney damage both in Type 1 and in Type 2 diabetes in mice. In leptin receptor-deficient db/db mice, a murine model of Type 2 diabetes, treatment with CCR2-blocking substances leads to reduced albuminuria [Okamoto et al., Biol. Pharm. Bull. 35 (11), 2069-2074 (2012); Sayyed et al., Kidney Int. 80, 68-78 (2011)]. In humans, too, accumulation of macrophages can be observed in diabetic nephropathy, and this correlates strongly with the progression of renal dysfunction [Kelly et al., Am. J. Nephrol. 32, 469-475 (2010); Nguyen et al., Nephrology 11, 226-231 (2006)]. Furthermore, the urine and plasma concentrations of MCP-1 in patients correlate with renal function and the stage of the chronic kidney disease [Eardley et al., Kidney Int. 69, 1189-1197 (2006); Stinghen et al., Nephron Clin. Pract. 111, c117-c126 (2009)], which suggests a critical role of macrophages in the pathogenesis of diabetic nephropathy.

Experimental data additionally confirm a reduction of reperfusion damage after renal ischaemia/reperfusion and reduced fibrosis in the unilateral ureteral obstruction (UUO) model in CCR2 knock-out animals [Furuichi et al., J. Am. Soc. Nephroi. 14, 2503-2515 (2003); Kitagawa et al., Am. J. Pathol. 165 (1), 237-246 (2004)].

It was therefore an object of the present invention to identify and provide novel substances which act as potent antagonists of the CCR2 receptor and are suitable as such for treatment and/or prevention of disorders, in particular cardiovascular, renal, inflammatory and fibrotic disorders.

The patent applications U.S. Pat. No. 2,628,236, EP 0 248 349-A2, EP 0 326 389-A2, WO 90/06918-A1, EP 0 407 342-A2, EP 0 514 192-A1, WO 93/08169-A1 and DE 4 493 151-T1 and the publications E. A. Falco et al., J. Am. Chem. Soc. 1951, 73, 3753-3758, ibid., 3758-3762 and V. V. Dovlatyan et al., Hayastani Kimiakan Handes 2003, 56 (1-2), 102-108 [Chem. Abstr. 140: 217586] disclose various 5-benzyl- and 5-phenoxypyrimidin-4-one derivatives as intermediates of preparation processes inter alia for pharmaceutically active compounds or active compounds for crop protection.

DE 1 695 270-A describes 2-amino-4-hydroxypyrimidines having fungicidal action. Hydroxypyrimidine and pyrimidinone derivatives having pharmacological activity which can be used for treating various disorders are disclosed, inter alia, in JP 06-220022-A [Chem. Abstr. 122:10058], WO 95/11235-A1, WO 2005/026148-A1, WO 2005/095381-A1, WO 2005/099688-A2, WO 2006/137840-A2, WO 2011/022440-A2, WO 2011/026835-A1 and WO 2014/058747-A1.

WO 2011/114148-A1 and WO 2012/041817-A1 recently described bicyclic pyrimidine derivatives as antagonists of the CCR2 receptor.

The present invention provides compounds of the general formula (I)

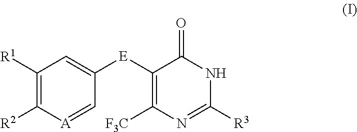

in which
A represents C—H, C—F or N,
E represents CH₂, CH(CH₃), O, S, S(═O) or S(═O)₂,
R¹ and R² independently of one another represent hydrogen, fluorine, chlorine, methyl, trifluoromethyl or trifluoromethoxy,
   where at least one of the two radicals R¹ and R² represents fluorine, chlorine, trifluoromethyl or trifluoromethoxy,
and
R³ represents (C₁-C₄)-alkyl which may be substituted by hydroxy, represents cyclopropyl or cyclobutyl or represents a group of the formula —NR^{4A}R^{4B}, —NH—C(═O)—R⁵, —NH—C(═O)—NH₂ or —CH₂—C(═O)—NH₂ in which
   R^{4A}, R^{4B} and R⁵ independently of one another represent hydrogen or (C₁-C₄)-alkyl,
and their salts, solvates and solvates of the salts.

Compounds according to the invention are the compounds of the formula (I) and their salts, solvates and solvates of the salts, the compounds encompassed by formula (I) of the formulae mentioned below and their salts, solvates and solvates of the salts and the compounds encompassed by formula (I) and mentioned below as working examples, and their salts, solvates and solvates of the salts, if the compounds encompassed by formula (I) and mentioned below are not already salts, solvates and solvates of the salts.

In the context of the present invention, preferred salts are physiologically acceptable salts of the inventive compounds. Also encompassed are salts which are not themselves suitable for pharmaceutical applications but can be used, for example, for the isolation, purification or storage of the compounds according to the invention.

Physiologically acceptable salts of the compounds according to the invention include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, benzenesulphonic acid, toluenesulphonic acid, naphthalenedisulphonic acid, formic acid, acetic acid, trifluoroacetic acid, propionic acid, succinic acid, fumaric acid, maleic acid, lactic acid, tartaric acid, malic acid, citric acid, gluconic acid, benzoic acid and embonic acid.

In the context of the invention, solvates refer to those forms of the inventive compounds which, in the solid or liquid state, form a complex by coordination with solvent molecules. Hydrates are a specific form of the solvates in which the coordination is with water. Solvates preferred in the context of the present invention are hydrates.

The inventive compounds may, depending on their structure, exist in different stereoisomeric forms, i.e. in the form of configurational isomers or else optionally as conformational isomers (enantiomers and/or diastereomers, including those in the case of atropisomers). The present invention therefore encompasses the enantiomers and diastereomers, and the respective mixtures thereof. The stereoisomerically homogeneous constituents can be isolated from such mixtures of enantiomers and/or diastereomers in a known manner; chromatography processes are preferably used for this purpose, especially HPLC chromatography on an achiral or chiral phase.

If the inventive compounds can occur in tautomeric forms, the present invention encompasses all the tautomeric forms.

In particular, the 6-(trifluoromethyl)pyrimidin-4(3H)-one derivatives of the formula (I) according to the invention may also be present in the tautomeric pyrimidin-4(1H)-one form (I′) or 4-hydroxypyrimidine form (I″) (see Scheme 1 below); these tautomeric forms are expressly embraced by the present invention.

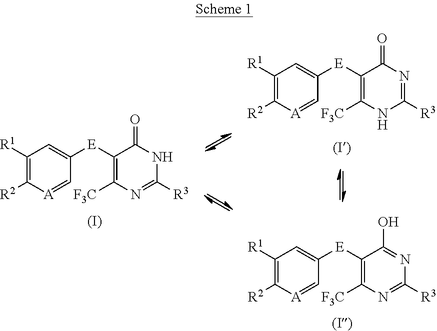

The present invention also encompasses all suitable isotopic variants of the inventive compounds. An isotopic variant of an inventive compound is understood here as meaning a compound in which at least one atom within the inventive compound has been exchanged for another atom of the same atomic number, but with a different atomic mass than the atomic mass which usually or predominantly occurs in nature. Examples of isotopes which can be incorporated into an inventive compound are those of hydrogen, carbon, nitrogen, oxygen, phosphorus, sulphur, fluorine, chlorine, bromine and iodine, such as ²H (deuterium), ³H (tritium), ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I. Particular isotopic variants of an inventive compound, especially those in which one or more radioactive isotopes have been incorporated, may be beneficial, for example, for the examination of the mechanism of action or of the active ingredient distribution in the body; due to comparatively easy preparability and detectability, especially compounds labelled with ³H or ¹⁴C isotopes are suitable for this purpose. In addition, the incorporation of isotopes, for example of deuterium, can lead to particular therapeutic benefits as a consequence of greater metabolic stability of the compound, for example to an extension of the half-life in the body or to a reduction in the active dose required; such modifications of the compounds according to the invention may therefore in some cases also constitute a preferred embodiment of the present invention. Isotopic variants of the compounds according to the invention can be prepared by generally customary processes known to those skilled in the art, for example by the methods described below and the procedures reported in the working examples, by using corresponding isotopic modifications of the particular reagents and/or starting compounds therein.

In addition, the present invention also encompasses prodrugs of the inventive compounds. The term “prodrugs” refers here to compounds which may themselves be biologically active or inactive, but are converted while present in the body, for example by a metabolic or hydrolytic route, to compounds according to the invention.

In the context of the present invention, unless specified otherwise, the substituents are defined as follows:

In the context of the invention, (C₁-C₄)-alkyl represents a straight-chain or branched alkyl radical having 1 to 4 carbon atoms. Preferred examples include: methyl, ethyl, n-propyl, isopropyl, n-butyl, isobutyl, sec-butyl and tert-butyl.

In the context of the present invention, it is the case that for all radicals which occur more than once, their meaning is independent of the others. When radicals in the compounds according to the invention are substituted, the radicals may be mono- or polysubstituted, unless specified otherwise.

Substitution by one or two identical or different substituents is preferred. Particular preference is given to substitution by one substituent.

In a particular embodiment, the present invention encompasses compounds of the formula (I) in which
A represents C—H, C—F or N,
E represents CH₂, O or S,
R¹ and R² independently of one another represent hydrogen, fluorine, chlorine, methyl or trifluoromethyl,
   where at least one of the two radicals R¹ and R² represents fluorine, chlorine or trifluoromethyl,
and
R³ represents (C₁-C₄)-alkyl which may be substituted by hydroxy, represents cyclopropyl or cyclobutyl or represents a group of the formula —NR^{4A}R^{4B}, —NH—C(═O)—R⁵, —NH—C(═O)—NH₂ or —CH₂—C(═O)—NH₂ in which
   R_{4A}, R^{4B} and R⁵ independently of one another represent hydrogen or (C₁-C₄)-alkyl,
and their salts, solvates and solvates of the salts.

Preference is given in the context of the present invention to compounds of the formula (I) in which
A represents C—H or C—F,
E represents CH₂, O or S,
R¹ represents fluorine, chlorine or trifluoromethyl,
R² represents hydrogen, fluorine, chlorine, methyl or trifluoromethyl
and
R³ represents (C₁-C₄)-alkyl which may be substituted by hydroxy, represents cyclopropyl or represents a group of the formula —NR^{4A}R^{4B} or —CH₂—C(═O)—NH₂ in which
   R^{4A} and R^{4B} each independently of one another represent hydrogen, methyl or ethyl,
and their salts, solvates and solvates of the salts.

In the context of the present invention, particular preference is given to compounds of the formula (I) in which
A represents C—H,
E represents CH₂ or O,
R¹ represents fluorine, chlorine or trifluoromethyl,
R² represents fluorine or chlorine
and
R³ represents methyl, hydroxymethyl, ethyl, n-propyl, cyclopropyl or a group of the formula —NR^{4A}R^{4B} or —CH₂—C(═O)—NH₂ in which

R^{4A} and R^{4B} both represent hydrogen, and their salts, solvates and solvates of the salts.

A particular embodiment of the present invention comprises compounds of the formula (I) in which
A represents C—H,
and their salts, solvates and solvates of the salts.

A further particular embodiment of the present invention comprises compounds of the formula (I) in which
E represents CH₂,
and their salts, solvates and solvates of the salts.

A further particular embodiment of the present invention comprises compounds of the formula (I) in which
E represents O,
and their salts, solvates and solvates of the salts.

A further particular embodiment of the present invention comprises compounds of the formula (I) in which
R¹ and R² each represent chlorine,
and their salts, solvates and solvates of the salts.

A further particular embodiment of the present invention comprises compounds of the formula (I) in which
R¹ represents trifluoromethyl
and
R² represents chlorine,
and their salts, solvates and solvates of the salts.

A further particular embodiment of the present invention comprises compounds of the formula (I) in which
R³ represents ethyl,
and their salts, solvates and solvates of the salts.

A further particular embodiment of the present invention comprises compounds of the formula (I) in which
R³ is cyclopropyl,
and their salts, solvates and solvates of the salts.

A further particular embodiment of the present invention comprises compounds of the formula (I) in which
R³ represents a group of the formula —NR^{4A}R^{4B} in which
   R^{4A} and R^{4B} both represent hydrogen,
and their salts, solvates and solvates of the salts.

A further particular embodiment of the present invention comprises compounds of the formula (I) in which
R³ represents a group of the formula —CH₂—C(═O)—NH₂,
and their salts, solvates and solvates of the salts.

The individual radical definitions specified in the particular combinations or preferred combinations of radicals are, independently of the particular combinations of the radicals specified, also replaced as desired by radical definitions of other combinations.

Very particular preference is given to combinations of two or more of the abovementioned preferred ranges.

The invention further provides a process for preparing the compounds according to the invention of the formula (I), characterized in that
[A] a compound of the formula (II)

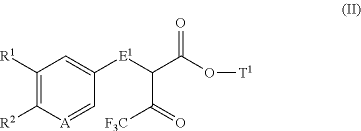

in which A, R¹ and R² have the meanings given above, represents CH₂ or O
and
T¹ represents methyl, ethyl, n-propyl or n-butyl
is condensed with a compound of the formula (III)

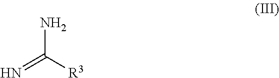

in which R³ has the meaning given above,
or a salt thereof to give a compound of the formula (I-A) according to the invention

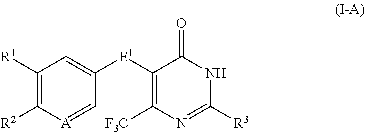

in which A, E¹, R¹, R² and R³ have the meanings given above
or
[B] a compound of the formula (IV)

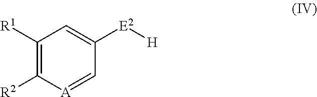

in which A, R¹ and R² have the meanings given above
and
E² represents O or S
is reacted in the form of an alkali metal salt or in the presence of a base with a compound of the formula (V)

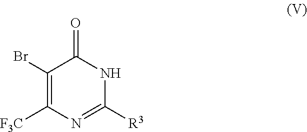

in which R³ has the meaning given above,
to give a compound of the formula (I-B) according to the invention

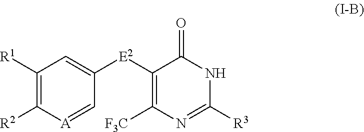

in which A, E², R¹, R² and R³ have the meanings given above
   and the resulting compounds of the formulae (I-A) and (I-B) are optionally converted with the appropriate (i) solvents and/or (ii) acids into their solvates, salts and/or solvates of the salts.

Suitable inert solvents for the process step (II)+(III)→(I-A) are, for example, alcohols such as methanol, ethanol, n-propanol, isopropanol, n-butanol or tert-butanol, ethers such as diethyl ether, diisopropyl ether, methyl tert-butyl ether, tetrahydrofuran, 1,4-dioxane, 1,2-dimethoxyethane or bis(2-methoxyethyl) ether, hydrocarbons or chlorinated hydrocarbons such as benzene, toluene, xylene or chlorobenzene, or dipolar aprotic solvents such as acetonitrile, butyronitrile, N,N-dimethylformamide (DMF), N,N-dimethylacetamide (DMA), dimethyl sulphoxide (DMSO), N,N′-dimethylpropyleneurea (DMPU) or N-methylpyrrolidinone (NMP). It is also possible to use mixtures of these solvents. Preference is given to using methanol, ethanol, 1,4-dioxane or N,N-dimethylformamide.

The compound of the formula (III) is preferably employed in the form of a salt, for example as hydrochloride, where in this case the reaction is carried out in the presence of an auxiliary base. Bases suitable for this purpose are in particular alkali metal hydroxides such as lithium hydroxide, sodium hydroxide or potassium hydroxide, alkali metal bicarbonates such as sodium bicarbonate or potassium bicarbonate, alkali metal carbonates such as lithium carbonate, sodium carbonate, potassium carbonate or caesium carbonate, alkali metal alkoxides such as sodium methoxide or potassium methoxide, sodium ethoxide or potassium ethoxide or sodium tert-butoxide or potassium tert-butoxide, or customary tertiary amine bases such as triethylamine, N-methylmorpholine, N-methylpiperidine, N,N-diisopropylethylamine, pyridine or 4-N,N-dimethylaminopyridine. The base used is preferably potassium carbonate, sodium methoxide or N,N-diisopropylethylamine.

The reaction (II)+(III)→(I-A) is generally carried out in a temperature range of from +20° C. to +150° C., preferably at from +60° C. to +120° C.

The process step (IV)+(V)→(I-B) is generally carried out in a temperature range of from +80° C. to +150° C. in a corresponding high-boiling inert solvent such as ethylene glycol, bis(2-methoxyethyl) ether, N,N-dimethylformamide (DMF), N,N-dimethylacetamide (DMA), dimethyl sulphoxide (DMSO), N,N′-dimethylpropyleneurea (DMPU) or N-methylpyrrolidinone (NMP). Preference is given to using ethylene glycol.

Suitable bases for this reaction are in particular alkali metal hydroxides such as lithium hydroxide, sodium hydroxide or potassium hydroxide, alkali metal carbonates such as lithium carbonate, sodium carbonate, potassium carbonate or caesium carbonate, alkali metal alkoxides such as sodium methoxide or potassium methoxide, sodium ethoxide or potassium ethoxide or sodium tert-butoxide or potassium tert-butoxide, or alkali metal hydrides such as sodium hydride or potassium hydride. Preference is given to using caesium carbonate.

The process steps described above can be carried out at atmospheric, elevated or reduced pressure (for example in the range from 0.5 to 5 bar); in general, the reactions are each carried out at atmospheric pressure.

For their part, the compounds of the formula (II) can be prepared by
[A-1] alkylating a trifluoroacetoacetic ester of the formula (VI)

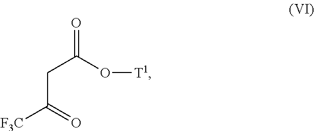

in which T¹ has the meaning given above,
in the presence of a base with a compound of the formula (VII)

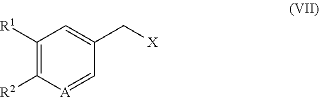

in which A, R¹ and R² have the meanings given above
and
X represents a leaving group, for example chlorine, bromine, iodine, mesylate, triflate or tosylate,
to give a compound of the formula (II-A)

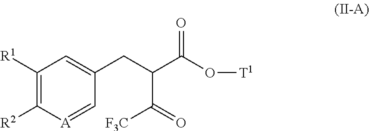

in which A, T¹, R¹ and R² have the meanings given above,
or
[A-2] acylating an aryloxyacetic ester of the formula (VIII)

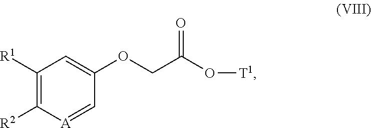

in which A, T¹, R¹ and R² have the meanings given above,
in the presence of a base with a trifluoroacetic ester of the formula (IX)

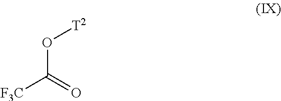

in which
T² represents methyl or ethyl,
to give a compound of the formula (II-B)

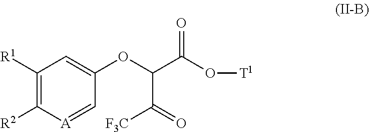

in which A, T¹, R¹ and R² have the meanings given above.

Inert solvents for the process step (VI)+(VII)→(II-A) are, for example, ethers such as diethyl ether, diisopropyl ether, methyl tert-butyl ether, tetrahydrofuran, 1,4-dioxane, 1,2-dimethoxyethane or bis(2-methoxyethyl) ether, or dipolar aprotic solvents such as acetone, methyl ethyl ketone, ethyl acetate, acetonitrile, butyronitrile, N,N-dimethylformamide (DMF), N,N-dimethylacetamide (DMA), dimethyl sulphoxide (DMSO), N-methylpyrrolidinone (NMP) or N,N′-dimethylpropyleneurea (DMPU). It is also possible to use mixtures of such solvents. Preference is given to using tetrahydrofuran.

Suitable bases for this reaction are in particular alkali metal carbonates such as sodium carbonate, potassium carbonate or caesium carbonate, alkali metal alkoxides such as sodium methoxide or potassium methoxide, sodium ethoxide or potassium ethoxide or sodium tert-butoxide or potassium tert-butoxide, alkali metal hydrides such as sodium hydride or potassium hydride, amides such as lithium bis(trimethylsilyl)amide or potassium bis(trimethylsilyl)amide or lithium diisopropylamide, or tertiary amine bases such as triethylamine, N-methylmorpholine, N-methylpiperidine, N,N-diisopropylethylamine, pyridine or 4-N,N-dimethylaminopyridine. The base used is preferably N,N-diisopropylethylamine

The reaction (VI)+(VII)→(II-A) is generally carried out in a temperature range of from 0° C. to +150° C., preferably from +20° C. to +100° C. Addition of an alkylation catalyst such as lithium chloride or lithium bromide, sodium iodide or potassium iodide, tetra-n-butylammonium bromide or benzyltriethylammonium chloride may optionally be advantageous.

Suitable inert solvents for the process step (VIII)+(IX)→(II-B) are, for example, alcohols such as methanol, ethanol, n-propanol, isopropanol, n-butanol or tert-butanol, ethers such as diethyl ether, diisopropyl ether, methyl tert-butyl ether, tetrahydrofuran, 1,4-dioxane, 1,2-dimethoxyethane or bis(2-methoxyethyl) ether, hydrocarbons or chlorinated hydrocarbons such as benzene, toluene, xylene or chlorobenzene, or dipolar aprotic solvents such as acetonitrile, butyronitrile, N,N-dimethylformamide (DMF), N,N-dimethylacetamide (DMA), dimethyl sulphoxide (DMSO), N,N′-dimethylpropyleneurea (DMPU) or N-methylpyrrolidinone (NMP). It is also possible to use mixtures of such solvents. Here, preference is given to using toluene.

Preferred bases for this reaction are alkali metal alkoxides such as sodium methoxide or potassium methoxide, sodium ethoxide or potassium ethoxide or sodium tert-butoxide or potassium tert-butoxide, alkali metal hydrides such as sodium hydride or potassium hydride, or amides such as lithium bis(trimethylsilyl)amide or potassium bis(trimethylsilyl)amide or lithium diisopropylamide. Preference is given to using sodium hydride.

The reaction (VIII)+(IX)→(II-B) is generally carried out in a temperature range of from 0° C. to +120° C.

The compounds of the formula (V) can be prepared by condensing, analogously to process [A], a trifluoroacetoacetic ester of the formula (VI)

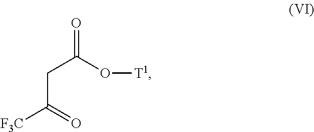

in which T¹ has the meaning given above,
with a compound of the formula (III)

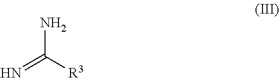

in which R³ has the meaning given above,
or a salt thereof to give a compound of the formula (X)

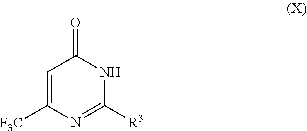

in which R³ has the meaning given above,
and then brominating the latter to give the compound of the formula (V).

The condensation reaction (VI)+(III)→(X) is carried out in a manner analogous to the reaction (II)+(III)→(I-A) described above in process [A]. Subsequent bromination of (X) to the compound (V) is preferably carried out with the aid of elemental bromine, N-bromosuccinimide (NBS) or 1,3-dibromo-5,5-dimethylhydantoin in an inert solvent such as dichloromethane, chloroform, tetrahydrofuran, acetonitrile, N,N-dimethylformamide (DMF) or acetic acid, within a temperature range of from −78° C. to +50° C.

The compounds of the formulae (III), (IV), (VI), (VII), (VIII) and (IX) are either commercially available or described as such in the literature, or they can be prepared from other commercially available compounds by generally customary methods known from the literature. Numerous detailed procedures and further literature references can also be found in the Experimental Part, in the section on the preparation of the starting compounds and intermediates.

The preparation of the compounds according to the invention can be illustrated in an exemplary manner by the Reaction Schemes 2-4 below:

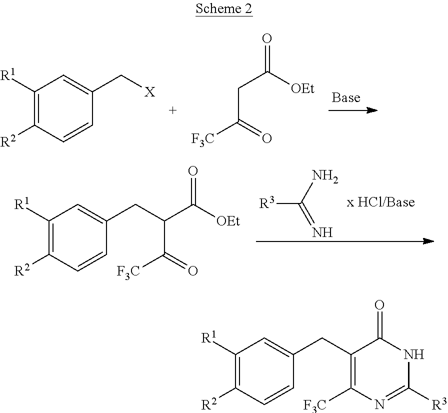

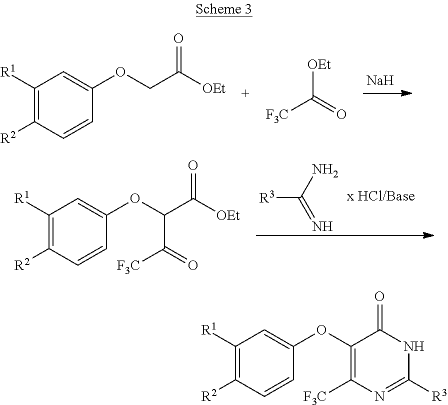

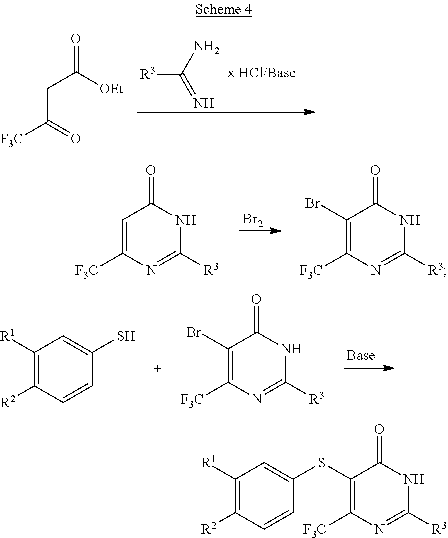

The compounds according to the invention have valuable pharmacological properties and can be used for prevention and treatment of diseases in humans and animals.

The compounds according to the invention are potent antagonists of the CCR2 receptor and are therefore particularly suitable for the treatment and/or prevention of disorders, in particular cardiovascular, renal, inflammatory, allergic and/or fibrotic disorders.

In the context of the present invention, cardiovascular disorders are understood to mean, for example, the following disorders: acute and chronic heart failure, arterial hypertension, coronary heart disease, acute coronary syndrome, myocardial infarction (STEMI, NSTEMI), acute myocardial infarction, stable and unstable angina pectoris, myocardial ischaemia, autoimmune heart disorders (pericarditis, endocarditis, valvolitis, aortitis, cardiomyopathies), shock, atherosclerosis, cardiac hypertrophy, cardiac fibrosis, atrial and ventricular arrhythmias, transitory and ischaemic attacks, stroke, pre-eclampsia, inflammatory cardiovascular disorders, peripheral and cardiac vascular disorders, peripheral perfusion disorders, arterial pulmonary hypertension, spasms of the coronary arteries and peripheral arteries, arterial and venous thromboses, thromboembolic disorders, oedema development, for example pulmonary oedema, cerebral oedema, renal oedema or heart failure-related oedema, restenoses, for example after thrombolysis treatments, percutaneous transluminal angioplasty (PTA), transluminal coronary angioplasty (PTCA), heart transplants and bypass operations, micro- and macrovascular damage (vasculitis), reperfusion damage, microalbuminuria, myocardial insufficiency, endothelial dysfunction, and also for the reduction in size of the myocardial region affected by myocardial infarction, and for the prevention of secondary infarctions.

In the context of the present invention, the term “heart failure” encompasses both acute and chronic forms of heart failure, and also more specific or related disease types thereof, such as acute decompensated heart failure, right heart failure, left heart failure, global failure, ischaemic cardiomyopathy, dilated cardiomyopathy, hypertrophic cardiomyopathy, idiopathic cardiomyopathy, congenital heart defects, heart valve defects, heart failure associated with heart valve defects, mitral valve stenosis, mitral valve insufficiency, aortic valve stenosis, aortic valve insufficiency, tricuspid valve stenosis, tricuspid valve insufficiency, pulmonary valve stenosis, pulmonary valve insufficiency, combined heart valve defects, myocardial inflammation (myocarditis), chronic myocarditis, acute myocarditis, viral myocarditis, diabetic heart failure, alcoholic cardiomyopathy, cardiac storage disorders, diastolic heart failure, systolic heart failure, and acute phases of worsening of existing heart failure (worsening heart failure).

In addition, the compounds according to the invention are suitable for treatment and/or prevention of renal disorders, especially of acute and chronic renal insufficiency, and of acute and chronic kidney failure.

In the context of the present invention, the term “acute renal insufficiency” encompasses acute manifestations of kidney disease, of kidney failure and/or renal insufficiency with and without the need for dialysis, and also underlying or related renal disorders such as renal hypoperfusion, ischaemic kidney disorders (AKI), intradialytic hypotension, volume deficiency (e.g. owing to dehydration or blood loss), shock, acute glomerulonephritis, haemolytic-uraemic syndrome (HUS), vascular catastrophe (arterial or venous thrombosis or embolism), cholesterol embolism, acute Bence-Jones kidney in the event of plasmacytoma, acute supravesicular or subvesicular efflux obstructions, immunological renal disorders such as kidney transplant rejection and immune complex-induced renal disorders, tubular dilatation, hyperphosphataemia, furthermore acute renal disorders which may be characterized by the need for dialysis, including in the case of partial resections of the kidney, dehydration through forced diuresis, uncontrolled blood pressure rise with malignant hypertension, urinary tract obstruction, urinary tract infection and amyloidosis, moreover systemic disorders with glomerular factors, such as rheumatological-immunological systemic disorders (e.g. lupus erythematodes), renal artery thrombosis, renal vein thrombosis, analgesic nephropathy and renal tubular acidosis, and X-ray contrast agent- or medicament-induced acute interstitial renal disorders.

In the context of the present invention, the term “chronic renal insufficiency” (CKD) encompasses chronic manifestations of kidney disease, of kidney failure and/or renal insufficiency with and without the need for dialysis, and also underlying or related renal disorders such as renal hypoperfusion, intradialytic hypotension, obstructive uropathy, glomerulopathy, glomerular and tubular proteinuria, renal oedema, haematuria, primary, secondary and chronic glomerulonephritis, membranous and membranoproliferative glomerulonephritis, Alport syndrome, glomerulosclerosis, tubulointerstitial disorders, nephropathic disorders such as primary and congenital kidney disease, renal inflammation, immunological renal disorders such as kidney transplant rejection, immune complex-induced renal disorders, diabetic and non-diabetic nephropathy, pyelonephritis, renal cysts, nephrosclerosis, hypertensive nephrosclerosis and nephrotic syndrome, which can be characterized diagnostically, for example, by abnormally reduced creatinine and/or water excretion, abnormally elevated blood concentrations of urea, nitrogen, potassium and/or creatinine, altered activity of renal enzymes, for example glutamyl synthetase, altered urine osmolarity or urine volume, elevated microalbuminuria, macroalbuminuria, glomerular and arteriolar lesions, tubular dilatation, hyperphosphataemia and/or the need for dialysis, and chronic renal disorders in the event of renal cell carcinoma, after partial resections of the kidney, in cases of dehydration through forced diuresis, uncontrolled blood pressure rise with malignant hypertension, urinary tract obstruction, urinary tract infection and amyloidosis, furthermore systemic disorders with glomerular factors, such as rheumatological-immunological systemic disorders (e.g. lupus erythematodes), renal artery stenosis, renal artery thrombosis, renal vein thrombosis, analgesic nephropathy, renal tubular acidosis, X-ray contrast agent- or medicament-induced chronic interstitial renal disorders and also in metabolic syndrome.

The present invention also comprises the use of the compounds according to the invention for the treatment and/or prevention of sequelae of renal insufficiency, for example pulmonary oedema, heart failure, uraemia, anaemia, electrolyte disturbances (for example hyperkalaemia, hyponatraemia) and disturbances in bone and carbohydrate metabolism.

The compounds according to the invention are further suitable for the treatment and/or prevention of polycystic kidney disease (PCKD) and of the syndrome of inappropriate ADH secretion (SIADH).

In addition, the compounds according to the invention are also suitable for treatment and/or prevention of pulmonary arterial hypertension (PAH) and other forms of pulmonary hypertension (PH), of chronic obstructive pulmonary disease (COPD), of acute respiratory distress syndrome (ARDS), of acute lung injury (ALI), pulmonary fibrosis, pulmonary emphysema (for example pulmonary emphysema caused by cigarette smoke), cystic fibrosis (CF), cardiogenic shock, aneurysms, sepsis (SIRS), multiple organ failure (MODS, MOF), inflammatory disorders of the kidney, chronic intestinal disorders (IBD, Crohn's Disease, ulcerative colitis), pancreatitis, peritonitis, rheumatoid disorders, inflammatory skin disorders and inflammatory eye disorders.

The compounds according to the invention can additionally be used for treatment and/or prevention of asthmatic disorders of varying severity with intermittent or persistent characteristics (refractive asthma, bronchial asthma, allergic asthma, intrinsic asthma, extrinsic asthma, medicament- or dust-induced asthma), of various forms of bronchitis (chronic bronchitis, infectious bronchitis, eosinophilic bronchitis), of Bronchiolitis obliterans, bronchiectasis, pneumonia, idiopathic interstitial pneumonia, farmer's lung and related disorders, of coughs and colds (chronic inflammatory cough, iatrogenic cough), inflammation of the nasal mucosa (including medicament-related rhinitis, vasomotoric rhinitis and seasonal allergic rhinitis, for example hay fever) and of polyps.

Furthermore, the compounds according to the invention are suitable for treatment and/or prevention of fibrotic disorders of the internal organs, for example the lung, the heart, the kidney, the bone marrow and in particular the liver, and also dermatological fibroses and fibrotic eye disorders. In the context of the present invention, the term “fibrotic disorders” encompasses particularly the following disorders: hepatic fibrosis, cirrhosis of the liver, pulmonary fibrosis, endomyocardial fibrosis, cardiomyopathy, nephropathy, glomerulonephritis, interstitial renal fibrosis, fibrotic damage resulting from diabetes, bone marrow fibrosis, peritoneal fibrosis and similar fibrotic disorders, scleroderma, amyotrophic lateral sclerosis (ALS), morphoea, keloids, hypertrophic scarring (also following surgical procedures), diabetic retinopathy and proliferative vitroretinopathy.

The compounds according to the invention can also be used for the treatment and/or prevention of metabolic disorders such as obesity and Type 2 diabetes, which are also accompanied by chronic inflammation, furthermore for the treatment and/or prevention of neurodegenerative disorders including Alzheimer's disease, multiple sclerosis and ischaemic brain damage, and also for pain, in particular neuropathic pain.

In addition, the compounds according to the invention can also be used for treatment and/or prevention of cancers (skin cancer, brain tumours, breast cancer, bone marrow tumours, leukaemias, liposarcomas, carcinoma of the gastrointestinal tract, of the liver, pancreas, lung, kidney, urinary tract, prostate and genital tract, and also malignant tumours in the lymphoproliferative system, for example Hodgkin's and non-Hodgkin's lymphoma), of disorders of the gastrointestinal tract and of the abdomen (glossitis, gingivitis, periodontitis, oesophagitis, eosinophilic gastroenteritis, mastocytosis, Crohn's disease, colitis, proctitis, pruritus ani, diarrhoea, coeliac disease, hepatitis, chronic hepatitis, hepatic fibrosis, cirrhosis of the liver, pancreatitis and cholecystitis), of skin disorders (allergic skin disorders, psoriasis, acne, eczema, neurodermitis, various forms of dermatitis, and also keratitis, bullosis, vasculitis, cellulitis, panniculitis, lupus erythematodes, erythema, lymphoma, skin cancer), of disorders of the skeletal bone and of the joints, and also of the skeletal muscle (various forms of arthritis and of arthropathies), and of further disorders with an inflammatory or immunological component, for example paraneoplastic syndrome, in the event of rejection reactions after organ transplants and for wound healing and angiogenesis, especially in the case of impaired wound healing and chronic wounds, for example diabetic foot ulcers and chronic venous leg ulcers.

The compounds according to the invention are additionally suitable for treatment and/or prevention of ophthalmologic disorders, for example glaucoma, age-related macular degeneration (AMD), of dry (non-exudative) AMD, wet (exudative, neovascular) AMD, choroidal neovascularization (CNV), diabetic retinopathy, atrophic changes to the retinal pigment epithelium (RPE), hypertrophic changes to the retinal pigment epithelium, macular oedema, diabetic macular oedema, retinal vein occlusion, choroidal retinal vein occlusion, macular oedema due to retinal vein occlusion, angiogenesis at the front of the eye, for example corneal angiogenesis, for example following keratitis, cornea transplant or keratoplasty, corneal angiogenesis due to hypoxia (as a result of extensive wearing of contact lenses), pterygium conjunctiva, subretinal oedema and intraretinal oedema. The compounds according to the invention are furthermore suitable for the treatment and/or prevention of elevated and high intraocular pressure as a result of traumatic hyphaema, periorbital oedema, postoperative viscoelastic retention or intraocular inflammation.

By virtue of their property profile, the compounds according to the invention are suitable in particular for the treatment and/or prevention of acute coronary syndrome, myocardial infarction, acute and chronic heart failure, acute and chronic kidney failure and acute lung damage.

The above-mentioned, well-characterized diseases in humans can also occur with a comparable aetiology in other mammals and can likewise be treated there with the compounds of the present invention.

In the context of the present invention, the term “treatment” or “treating” includes inhibition, retardation, checking, alleviating, attenuating, restricting, reducing, suppressing, repelling or healing of a disease, a condition, a disorder, an injury or a health problem, or the development, the course or the progression of such states and/or the symptoms of such states. The term “therapy” is understood here to be synonymous with the term “treatment”.

The terms “prevention”, “prophylaxis” or “preclusion” are used synonymously in the context of the present invention and refer to the avoidance or reduction of the risk of contracting, experiencing, suffering from or having a disease, a condition, a disorder, an injury or a health problem, or a development or advancement of such states and/or the symptoms of such states.

The treatment or prevention of a disease, a condition, a disorder, an injury or a health problem may be partial or complete.

The present invention thus further provides for the use of the compounds according to the invention for the treatment and/or prevention of disorders, in particular the disorders mentioned above.

The present invention further provides for the use of the compounds according to the invention for producing a medicament for the treatment and/or prevention of disorders, in particular the disorders mentioned above.

The present invention further provides a medicament comprising at least one of the compounds according to the invention, for the treatment and/or prevention of disorders, in particular the disorders mentioned above.

The present invention furthermore provides for the use of the compounds according to the invention in a method for treatment and/or prevention of disorders, in particular the disorders mentioned above.

The present invention further provides a method for treatment and/or prevention of disorders, in particular the disorders mentioned above, using an effective amount of at least one of the compounds according to the invention.

The compounds according to the invention can be used alone or, if required, in combination with one or more other pharmacologically active substances, provided that this combination does not lead to undesirable and unacceptable side effects. The present invention furthermore therefore provides medicaments containing at least one of the compounds according to the invention and one or more further active compounds, in particular for treatment and/or prevention of the abovementioned disorders. Preferred examples of active compounds suitable for combinations include:
compounds which inhibit the signal transduction cascade, by way of example and with preference from the group of the kinase inhibitors, especially from the group of the tyrosine kinase and/or serine/threonine kinase inhibitors;
compounds which inhibit the degradation and alteration of the extracellular matrix, by way of example and with preference inhibitors of the matrix metalloproteases (MMPs), especially inhibitors of stromelysin, collagenases, gelatinases and aggrecanases (in this context particularly of MMP-1, MMP-3, MMP-8, MMP-9, MMP-10, MMP-11 and MMP-13) and of metalloelastase (MMP-12);
compounds which block the binding of serotonin to its receptors, by way of example and with preference antagonists of the 5-HT2B receptor such as PRX-08066;
organic nitrates and NO donors, for example sodium nitroprusside, nitroglycerin, isosorbide mononitrate, isosorbide dinitrate, molsidomine or SIN-1, and inhaled NO;
NO-independent but haem-dependent stimulators of guanylate cyclase, such as especially riociguat and the compounds described in WO 00/06568, WO 00/06569, WO 02/42301, WO 03/095451, WO 2011/147809, WO 2012/004258, WO 2012/028647 and WO 2012/059549;
NO- and haem-independent activators of soluble guanylate cyclase, such as especially the compounds described in WO 01/19355, WO 01/19776, WO 01/19778, WO 01/19780, WO 02/070462 and WO 02/070510;
compounds which inhibit the degradation of cyclic guanosine monophosphate (cGMP) and/or cyclic adenosine monophosphate (cAMP), for example inhibitors of phosphodiesterases (PDE) 1, 2, 3, 4 and/or 5, especially PDE 5 inhibitors such as sildenafil, vardenafil, tadalafil, udenafil, dasantafil, avanafil, mirodenafil or lodenafil;
prostacyclin analogues and IP receptor agonists, by way of example and with preference iloprost, beraprost, treprostinil, epoprostenol or NS-304;
bronchodilatory agents, by way of example and with preference from the group of the beta-adrenergic receptor agonists, such as especially albuterol, isoproterenol, metaproterenol, terbutalin, fenoterol, formoterol, reproterol, salbutamol or salmeterol, and from the group of the anticholinergics, such as especially ipratropium bromide, tiotropium bromide or oxitropium bromide;
anti-inflammatory agents, by way of example and with preference from the group of the glucocorticoids, such as especially prednisone, prednisolone, methylprednisolone, triamcinolone, dexamethasone, beclomethasone, betamethasone, flunisolide, budesonide or fluticasone;
compounds which inhibit soluble epoxide hydrolase (sEH), for example N,N′-dicyclohexylurea, 12-(3-adamantan-1-ylureido)dodecanoic acid or 1-adamantan-1-yl-3-{5-[2-(2-ethoxyethoxy)ethoxy]pentyl}urea;
compounds which influence the energy metabolism of the heart, by way of example and with preference etomoxir, dichloroacetate, ranolazine or trimetazidine;
vasopressin receptor antagonists, for example and with preference conivaptan, tolvaptan, lixivaptan, mozavaptan, satavaptan, SR-121463, RWJ-676070 or BAY 86-8050;
antihyperglycaemic agents (antidiabetics), by way of example and with preference from the group of the biguanides such as metformin, of the sulphonylureas, such as glibenclamide or glimepiride, of the glinides, such as repaglinide or nateglinide, of the DPP IV inhibitors, such as sitagliptin, vildagliptin or saxagliptin, of the glucosidase inhibitors, such as acarbose or miglitol, and of the amyline analogues, such as pramlintide;
hypotensive active ingredients, for example and with preference from the group of calcium antagonists, angiotensin AII antagonists, ACE inhibitors, vasopeptidase inhibitors, endothelin antagonists, renin inhibitors, alpha-receptor blockers, beta-receptor blockers, mineralocorticoid receptor antagonists, and rho kinase inhibitors and the diuretics;
agents having antithrombotic activity, for example and with preference from the group of the platelet aggregation inhibitors, the anticoagulants and the profibrinolytic substances; and/or
active compounds which alter lipid metabolism, for example and with preference from the group of thyroid receptor agonists, cholesterol synthesis inhibitors, preferred examples being HMG-CoA reductase inhibitors or squalene synthesis inhibitors, of ACAT inhibitors, CETP inhibitors, MTP inhibitors, PPAR-alpha, PPAR-gamma and/or PPAR-delta agonists, cholesterol absorption inhibitors, lipase inhibitors, polymeric bile acid adsorbents, bile acid reabsorption inhibitors and lipoprotein(a) antagonists.

In a preferred embodiment of the invention, the compounds according to the invention are employed in combination with a kinase inhibitor, by way of example and with preference nintedanib, dasatinib, nilotinib, bosutinib, regorafenib, sorafenib, sunitinib, cediranib, axitinib, telatinib, imatinib, brivanib, pazopanib, vatalanib, gefitinib, erlotinib, lapatinib, canertinib, lestaurtinib, lonafarnib, pelitinib, semaxanib, tandutinib or tipifarnib.

Hypotensive agents are preferably understood to mean compounds from the group of calcium antagonists, angiotensin AII antagonists, ACE inhibitors, endothelin antagonists, renin inhibitors, alpha-receptor blockers, beta-receptor blockers, mineralocorticoid receptor antagonists, rho kinase inhibitors, and the diuretics.

In a preferred embodiment of the invention, the compounds according to the invention are administered in combination with a calcium antagonist, by way of example and with preference nifedipine, amlodipine, verapamil or diltiazem.

In a preferred embodiment of the invention, the compounds according to the invention are administered in combination with an alpha-1-receptor blocker, by way of example and with preference prazosin.

In a preferred embodiment of the invention, the compounds according to the invention are administered in combination with a beta-receptor blocker, by way of example and with preference propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the compounds according to the invention are administered in combination with an angiotensin AII antagonist, by way of example and with preference losartan, candesartan, valsartan, telmisartan or embusartan.

In a preferred embodiment of the invention, the compounds according to the invention are administered in combination with an ACE inhibitor, by way of example and with preference enalapril, captopril, lisinopril, ramipril, delapril, fosinopril, quinopril, perindopril or trandopril.

In a preferred embodiment of the invention, the compounds according to the invention are administered in combination with an endothelin antagonist, by way of example and with preference bosentan, darusentan, ambrisentan or sitaxsentan.

In a preferred embodiment of the invention, the compounds according to the invention are administered in combination with a renin inhibitor, by way of example and with preference aliskiren, SPP-600 or SPP-800.

In a preferred embodiment of the invention, the compounds according to the invention are administered in combination with a mineralocorticoid receptor antagonist, by way of example and with preference spironolactone or eplerenone.

In a preferred embodiment of the invention, the compounds according to the invention are administered in combination with a rho kinase inhibitor, by way of example and with preference fasudil, Y-27632, SLx-2119, BF-66851, BF-66852, BF-66853, KI-23095, SB-772077, GSK-269962A or BA-1049.

In a preferred embodiment of the invention, the compounds according to the invention are administered in combination with a diuretic, preferred examples being furosemide, bumetanide, torsemide, bendroflumethiazide, chlorthiazide, hydrochlorthiazide, hydroflumethiazide, methyclothiazide, polythiazide, trichlormethiazide, chlorthalidone, indapamide, metolazone, quinethazone, acetazolamide, dichlorophenamide, methazolamide, glycerol, isosorbide, mannitol, amiloride or triamterene.

Antithrombotic agents are preferably understood to mean compounds from the group of the platelet aggregation inhibitors, the anticoagulants and the profibrinolytic substances.

In a preferred embodiment of the invention, the compounds according to the invention are administered in combination with a platelet aggregation inhibitor, by way of example and with preference aspirin, clopidogrel, ticlopidin or dipyridamole.

In a preferred embodiment of the invention, the compounds according to the invention are administered in combination with a thrombin inhibitor, by way of example and with preference ximelagatran, melagatran, dabigatran, bivalirudin or clexane.

In a preferred embodiment of the invention, the compounds according to the invention are administered in combination with a GPIIb/IIIa antagonist such as, by way of example and with preference, tirofiban or abciximab.

In a preferred embodiment of the invention, the compounds according to the invention are administered in combination with a factor Xa inhibitor, by way of example and with preference rivaroxaban, apixaban, edoxaban, razaxaban, fondaparinux, idraparinux, DU-176b, PMD-3112, YM-150, KFA-1982, EMD-503982, MCM-17, MLN-1021, DPC 906, JTV 803, SSR-126512 or SSR-128428.

In a preferred embodiment of the invention, the compounds according to the invention are administered in combination with heparin or with a low molecular weight (LMW) heparin derivative.

In a preferred embodiment of the invention, the compounds according to the invention are administered in combination with a vitamin K antagonist, by way of example and with preference coumarin.

Lipid metabolism modifiers are preferably understood to mean compounds from the group of the CETP inhibitors, thyroid receptor agonists, cholesterol synthesis inhibitors such as HMG-CoA reductase inhibitors or squalene synthesis inhibitors, the ACAT inhibitors, MTP inhibitors, PPAR-alpha, PPAR-gamma and/or PPAR-delta agonists, cholesterol absorption inhibitors, polymeric bile acid adsorbents, bile acid reabsorption inhibitors, lipase inhibitors and the lipoprotein(a) antagonists.

In a preferred embodiment of the invention, the compounds according to the invention are administered in combination with a CETP inhibitor, by way of example and with preference torcetrapib (CP-529 414), JJT-705 or CETP vaccine (Avant).

In a preferred embodiment of the invention, the compounds according to the invention are administered in combination with a thyroid receptor agonist, by way of example and with preference D-thyroxin, 3,5,3′-triiodothyronin (T3), CGS 23425 or axitirome (CGS 26214).

In a preferred embodiment of the invention, the compounds according to the invention are administered in combination with an HMG-CoA reductase inhibitor from the class of statins, by way of example and with preference lovastatin, simvastatin, pravastatin, fluvastatin, atorvastatin, rosuvastatin or pitavastatin.

In a preferred embodiment of the invention, the compounds according to the invention are administered in combination with a squalene synthesis inhibitor, by way of example and with preference BMS-188494 or TAK-475.

In a preferred embodiment of the invention, the compounds according to the invention are administered in combination with an ACAT inhibitor, by way of example and with preference avasimibe, melinamide, pactimibe, eflucimibe or SMP-797.

In a preferred embodiment of the invention, the compounds according to the invention are administered in combination with an MTP inhibitor, by way of example and with preference implitapide, BMS-201038, R-103757 or ITT-130.

In a preferred embodiment of the invention, the compounds according to the invention are administered in combination with a PPAR-gamma agonist, by way of example and with preference pioglitazone or rosiglitazone.

In a preferred embodiment of the invention, the compounds according to the invention are administered in combination with a PPAR-delta agonist, by way of example and with preference GW 501516 or BAY 68-5042.

In a preferred embodiment of the invention, the compounds according to the invention are administered in combination with a cholesterol absorption inhibitor, by way of example and with preference ezetimibe, tiqueside or pamaqueside.

In a preferred embodiment of the invention, the compounds according to the invention are administered in combination with a lipase inhibitor, by way of example and with preference orlistat.

In a preferred embodiment of the invention, the compounds according to the invention are administered in combination with a polymeric bile acid adsorbent, by way of example and with preference cholestyramine, colestipol, colesolvam, CholestaGel or colestimide.

In a preferred embodiment of the invention, the compounds according to the invention are administered in combination with a bile acid reabsorption inhibitor, by way of example and with preference ASBT (═IBAT) inhibitors, for example AZD-7806, S-8921, AK-105, BARI-1741, SC-435 or SC-635.

In a preferred embodiment of the invention, the compounds according to the invention are administered in combination with a lipoprotein(a) antagonist, by way of example and with preference gemcabene calcium (CI-1027) or nicotinic acid.

Particular preference is given to combinations of the compounds according to the invention with one or more further active compounds selected from the group of the antihyperglycaemic agents (antidiabetics), the hypotensive agents, the platelet aggregation inhibitors, the anticoagulants and the HMG-CoA reductase inhibitors (statins).

The present invention further provides medicaments which comprise at least one compound according to the invention, typically together with one or more inert, nontoxic, pharmaceutically suitable excipients, and the use thereof for the aforementioned purposes.

The inventive compounds may act systemically and/or locally. For this purpose, they can be administered in a suitable manner, for example by the oral, parenteral, pulmonal, nasal, sublingual, lingual, buccal, rectal, dermal, transdermal, conjunctival or otic route, or as an implant or stent.

The inventive compounds can be administered in suitable administration forms for these administration routes.

Suitable administration forms for oral administration are those which work according to the prior art and release the compounds according to the invention rapidly and/or in a modified manner and which contain the compounds according to the invention in crystalline and/or amorphized and/or dissolved form, for example tablets (uncoated or coated tablets, for example with gastric juice-resistant or retarded-dissolution or insoluble coatings which control the release of the compound according to the invention), tablets or films/oblates which disintegrate rapidly in the oral cavity, films/lyophilizates, capsules (for example hard or soft gelatin capsules), sugar-coated tablets, granules, pellets, powders, emulsions, suspensions, aerosols or solutions.

Parenteral administration can bypass an absorption step (e.g. intravenously, intraarterially, intracardially, intraspinally or intralumbally) or include an absorption (e.g. inhalatively, intramuscularly, subcutaneously, intracutaneously, percutaneously or intraperitoneally). Suitable administration forms for parenteral administration include injection and infusion formulations in the form of solutions, suspensions, emulsions, lyophilizates or sterile powders.

For the other administration routes, suitable examples are inhalable medicament forms (including powder inhalers, nebulizers, metered aerosols), nasal drops, solutions or sprays, tablets, films/oblates or capsules for lingual, sublingual or buccal administration, suppositories, ear or eye preparations, vaginal capsules, aqueous suspensions (lotions, shaking mixtures), lipophilic suspensions, ointments, creams, transdermal therapeutic systems (e.g. patches), milk, pastes, foams, sprinkling powders, implants or stents.

Preference is given to oral and intravenous administration.

The inventive compounds can be converted to the administration forms mentioned. This can be accomplished in a manner known per se by mixing with inert, non-toxic, pharmaceutically suitable excipients. These excipients include carriers (for example microcrystalline cellulose, lactose, mannitol), solvents (e.g. liquid polyethylene glycols), emulsifiers and dispersing or wetting agents (for example sodium dodecylsulphate, polyoxysorbitan oleate), binders (for example polyvinylpyrrolidone), synthetic and natural polymers (for example albumin), stabilizers (e.g. antioxidants, for example ascorbic acid), colorants (e.g. inorganic pigments, for example iron oxides) and flavour and/or odour correctants.

In general, it has been found to be advantageous in the case of parenteral administration to administer amounts of from about 0.001 to 5 mg/kg, preferably about 0.01 to 3 mg/kg, of body weight to achieve effective results. In the case of oral administration the dosage is about 0.01 to 100 mg/kg, preferably about 0.01 to 50 mg/kg and most preferably 0.1 to 30 mg/kg of body weight. In the case of intrapulmonary administration, the amount is generally about 0.1 to 50 mg per inhalation.

It may nevertheless be necessary where appropriate to deviate from the stated amounts, specifically as a function of the body weight, route of administration, individual response to the active ingredient, nature of the preparation and time or interval over which administration takes place. Thus, in some cases less than the abovementioned minimum amount may be sufficient, while in other cases the upper limit mentioned must be exceeded. In the case of administration of greater amounts, it may be advisable to divide them into several individual doses over the day.

The working examples which follow illustrate the invention. The invention is not restricted to the examples.

### A. EXAMPLES

### Abbreviations and Acronyms

abs. absolute
Ac acetyl
aq. aqueous, aqueous solution
br. broad (in NMR signal)
Ex. Example
Bu butyl
c concentration
cat. catalytic
CI chemical ionization (in MS)
d doublet (in NMR)
d day(s)
TLC thin-layer chromatography
DCI direct chemical ionization (in MS)
dd doublet of doublets (in NMR)
DIPEA N,N-diisopropylethylamine
DMAP 4-N,N-dimethylaminopyridine
DME 1,2-dimethoxy ethane
DMF N,N-dimethylformamide
DMSO dimethyl sulphoxide
dt doublet of triplets (in NMR)
ee enantiomeric excess
EI electron impact ionization (in MS)
ent enantiomerically pure, enantiomer
eq. equivalent(s)
ES electrospray ionization (in MS)
Et ethyl
GC gas chromatography
GC-MS gas chromatography-coupled mass spectrometry
h hour(s)
HPLC high-pressure high-performance liquid chromatography
iPr isopropyl
conc. concentrated (in the case of a solution)
LC liquid chromatography
LC-MS liquid chromatography-coupled mass spectrometry
lit. literature (reference)
m multiplet (in NMR)
Me methyl
min minute(s)
MPLC medium-pressure liquid chromatography (on silica gel; also referred to as flash chromatography)
Ms methanesulphonyl (mesyl)
MS mass spectrometry
NMP N-methyl-2-pyrrolidinone
NMR nuclear magnetic resonance spectrometry
Pd/C palladium on activated carbon
PEG polyethylene glycol
Pr propyl
prep. preparative
q (or quart) quartet (in NMR)
qd quartet of doublets (in NMR)
quant. quantitative (in chemical yield)
quint quintet (in NMR)
rac racemic, racemate
Rf retention index (in TLC)
RP reversed phase (in HPLC)
RT room temperature
R_{f} retention time (in HPLC, LC/MS)
s singlet (in NMR)
sept septet (in NMR)
t triplet (in NMR)
tBu tert-butyl
td triplet of doublets (in NMR)
Tf trifluoromethylsulphonyl (triflyl)
TFA trifluoroacetic acid
THF tetrahydrofuran
Ts para-tolylsulphonyl (tosyl)
UV ultraviolet spectrometry
v/v ratio by volume (of a solution)
tog. together
   LC-MS methods:

### Method 1 (LC-MS):

Instrument: Waters ACQUITY SQD UPLC System; column: Waters Acquity UPLC HSS T3 1.8μ, 50×1 mm; mobile phase A: 1 l of water+0.25 ml of 99% formic acid, eluent B: 1 l of acetonitrile+0.25 ml of 99% formic acid; gradient: 0.0 min 90% A→1.2 min 5% A→2.0 min 5% A; oven: 50° C.; flow rate: 0.40 ml/min; UV detection: 208-400 nm.

### Method 2 (LC-MS):

Instrument: Waters ACQUITY SQD UPLC System; column: Waters Acquity UPLC HSS T3 1.8μ, 50×1 mm; mobile phase A: 1 l of water+0.25 ml of 99% formic acid, eluent B: 1 l of acetonitrile+0.25 ml of 99% formic acid; gradient: 0.0 min 95% A→6.0 min 5% A→7.5 min 5% A; oven: 50° C.; flow rate: 0.35 ml/min; UV detection: 210-400 nm.

### Method 3 (LC-MS):

Instrument: Micromass Quattro Premier with Waters UPLC Acquity; column: Thermo Hypersil GOLD 1.9 50×1 mm; mobile phase A: 1 l of water+0.5 ml of 50% strength formic acid, mobile phase B: 1 l of acetonitrile+0.5 ml of 50% formic acid; gradient: 0.0 min 97% A→0.5 min 97% A→3.2 min 5% A→4.0 min 5% A; oven: 50° C.; flow rate: 0.3 ml/min; UV detection: 210 nm.

### Method 4 (LC-MS):

MS instrument: Waters Micromass QM; HPLC instrument: Agilent 1100 series; column: Agilent ZORBAX Extend-C18 3.5μ, 3.0×50 mm; mobile phase A: 1 l of water+0.01 mol of ammonium carbonate, mobile phase B: 1 l of acetonitrile; gradient: 0.0 min 98% A→0.2 min 98% A→3.0 min 5% A→4.5 min 5% A; oven: 40° C.; flow rate: 1.75 ml/min; UV detection: 210 nm.

### Method 5 (LC-MS):

MS instrument: Waters Micromass ZQ; HPLC instrument: Agilent 1100 series; column: Agilent ZORBAX Extend-C18 3.5 3.0×50 mm; mobile phase A: 1 l of water+0.01 mol of ammonium carbonate, mobile phase B: 1 l of acetonitrile; gradient: 0.0 min 98% A→0.2 min 98% A→3.0 min 5% A→4.5 min 5% A; oven: 40° C.; flow rate: 1.75 ml/min; UV detection: 210 nm.

### Method 6 (LC-MS):

Instrument: Agilent MS Quad 6150; HPLC: Agilent 1290; column: Waters Acquity UPLC HSS T3 1.8μ, 50×2.1 mm; mobile phase A: 1 l of water+0.25 ml of 99% formic acid, eluent B: 1 l of acetonitrile+0.25 ml of 99% formic acid; gradient: 0.0 min 90% A→0.3 min 90% A→1.7 min 5% A→3.0 min 5% A; oven: 50° C.; flow rate: 1.20 ml/min; UV detection: 205-305 nm.

### Further Details:

The percentages in the example and test descriptions which follow are, unless indicated otherwise, percentages by weight; parts are parts by weight. Solvent ratios, dilution ratios and concentration data for the liquid/liquid solutions are in each case based on volume.

Purities are generally based on corresponding peak integrations in the LC/MS chromatogram, but they may additionally have been determined with the aid of the ¹H-NMR spectrum. If no purity is indicated, the purity is generally 100% according to automated peak integration in the LC/MS chromatogram, or the purity has not been determined explicitly.

Stated yields in % of theory are generally corrected for purity if a purity of <100% is indicated. In solvent-containing or impure batches, the formal yield may be “>100%”; in these cases the yield is not corrected for solvent or purity.

When compounds according to the invention are purified by preparative HPLC, where the mobile phases contain additives such as, for example, trifluoroacetic acid, formic acid or ammonia, the compounds according to the invention may be obtained in salt form, for example as trifluoroacetate, formate or ammonium salt, if the compounds according to the invention have a sufficiently basic or acidic functionality. Such a salt can be converted to the corresponding free base or acid by various methods known to the person skilled in the art.

Some of the descriptions below of the coupling patterns of ¹H-NMR signals were taken directly from the suggestions of the ACD SpecManager (ACD/Labs Release 12.00, Product version 12.5) and have not necessarily been rigorously checked. In some cases, the suggestions of the SpecManager were adjusted manually Manually adjusted or assigned descriptions are generally based on the optical appearance of the signals in question and do not necessarily correspond to a strict, physically correct interpretation. In general, the stated chemical shift refers to the centre of the signal in question. In the case of broad multiplets, an interval is given. Signals obscured by solvent or water were either tentatively assigned or have not been listed.

Melting points and melting-point ranges, if stated, are uncorrected.

All reactants or reagents whose preparation is not described explicitly hereinafter were purchased commercially from generally accessible sources. For all other reactants or reagents whose preparation likewise is not described hereinafter and which were not commercially obtainable or were obtained from sources which are not generally accessible, a reference is given to the published literature in which their preparation is described.

### Starting Materials and Intermediates

### Example 1A

### Ethyl 2-[4-chloro-3-(trifluoromethyl)phenoxy]acetate

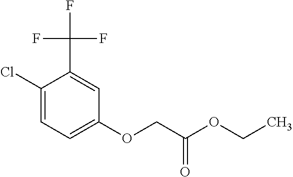 [SMILES: CCOC(=O)COc1ccc(Cl)c(C(F)(F)F)c1]

At 23° C. (cooling !), 25 g (127.2 mmol) of 4-chloro-3-(trifluoromethyl)phenol in 50 ml of THF were added dropwise to a suspension of 5.6 g (140 mmol) of sodium hydride (60% in paraffin) in 125 ml of THF, with evolution of hydrogen in an exothermic reaction. After 30 min of stirring, 23.4 g (140 mmol) of ethyl bromoacetate in 50 ml of THF were added dropwise, and the mixture was stirred at 23° C. for 2 h. Another 2.34 g of ethyl bromoacetate were added, and the mixture was stirred at 23° C. for a further 2 h. The mixture was then diluted with ethyl acetate and washed with water, and the aqueous phase was re-extracted with ethyl acetate. The combined organic phases were washed with water and dried over sodium sulphate. After removal of the drying agent by filtration, the mixture was concentrated under reduced pressure. Drying under high vacuum gave 38.3 g (96% of theory, purity 90%) of the target compound. The product could be converted further without further purification.

LC-MS (Method 1): Rₜ=1.15 min; MS (ESneg): not ionizable

¹H NMR (400 MHz, DMSO-d₆): δ=1.21 (t, 3H), 4.17 (q, 2H), 4.94 (s, 2H), 7.29 (dd, 1H), 7.37 (d, 1H), 7.64 (d, 1H).

### Example 2A

### Ethyl 2-[4-fluoro-3-(trifluoromethyl)phenoxy]acetate

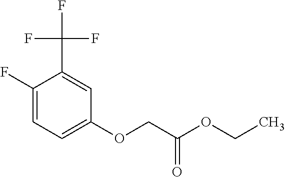 [SMILES: CCOC(=O)COc1ccc(F)c(C(F)(F)F)c1]

At 23° C., 2 g (11.1 mmol) of 4-fluoro-3-(trifluoromethyl)phenol were added dropwise to a suspension of 0.49 g (12.2 mmol) of sodium hydride (60% in paraffin) in 25 ml of THF, with evolution of hydrogen in an exothermic reaction. After 30 min of stirring, 1.86 g (11.1 mmol) of ethyl bromoacetate were added, and the mixture was stirred at 23° C. for 18 h. The mixture was then diluted with ethyl acetate and washed with water, and the organic phase was dried over magnesium sulphate. After removal of the drying agent by filtration, the mixture was concentrated under reduced pressure. Drying under high vacuum gave 2.43 g (78% of theory, purity 95%) of the target compound.

LC-MS (Method 3): Rₜ=2.42 min; MS (ESpos): m/z=267 (M+H)⁺.

The following compounds are known from the literature, commercially available or can be prepared analogously to Example 2A:

TABLE 1

| Example | | |
|---|---|---|
| No. | IUPAC name/structure | CAS number; literature |
| 3A | ethyl (4-chloro-3-fluorophenoxy)acetate 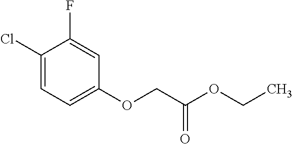 | CAS 1096703-33-1; preparation described in WO 2012/041817 (Intermediate 87) |
| 4A | ethyl (3-chloro-4-fluorophenoxy)acetate 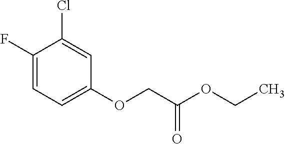 | CAS 667437-18-5; preparation described in Tetrahedron 2004, 60 (52), 12231- 12237 |
| 5A | ethyl (3,4-difluorophenoxy)acetate 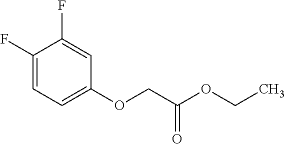 | CAS 1094524-83-0 |
| 6A | ethyl (3-chlorophenoxy)acetate 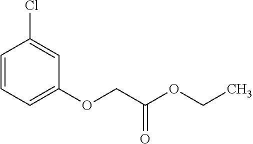 | CAS 52094-98-1; commercially available |
| 7A | ethyl 2-[(5-chloropyridin-3-yl)oxy]acetate 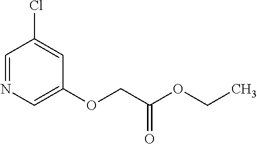 | CAS 53233-36-6; commercially available |
| 8A | ethyl (3,4-dichlorophenoxy)acetate 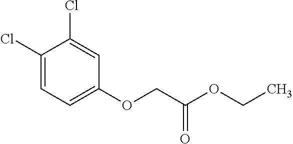 | CAS 62855-72-5; preparation described in 2012/041817 (Intermediate 88) |

### Example 9A

### Ethyl 2-14-chloro-3-(trifluoromethyl)phenoxyl-4,4,4-trifluoro-3-oxobutanoate

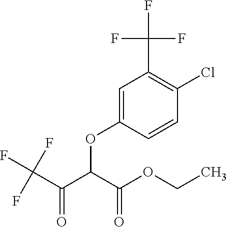 [SMILES: CCOC(=O)C(Oc1ccc(Cl)c(C(F)(F)F)c1)C(=O)C(F)(F)F]

Initially 26 g (182.8 mmol) of ethyl trifluoroacetate and then 38.3 g (121.9 mmol, purity 90%) of ethyl [4-chloro-3-(trifluoromethyl)phenoxy]acetate were added dropwise to a suspension of 12.19 g (304.7 mmol) of sodium hydride (60% in paraffin) in 150 ml of toluene. The mixture was heated to reflux, resulting in a noticeable evolution of gas, and boiled for one hour. The cooled reaction was then acidified with 1 N hydrochloric acid. The organic phase was separated off, washed with dilute brine, dried over sodium sulphate and filtered, and the filtrate was concentrated. Drying under high vacuum gave 50.6 g (76% of theory, purity 69%) of the target compound. The product was converted further without further purification.

LC-MS (Method 3): Rₜ=2.51 min; MS (ESneg): m/z=377 (M−H)⁻.

The following synthesis intermediates were prepared analogously to Example 9A:

TABLE 2

| Example | IUPAC name/structure | |
|---|---|---|
| No. | (yield; reaction time) | Analytical data |
| 10A | ethyl 2-(4-chloro-3-fluorophenoxy)-4,4,4- trifluoro-3-oxobutanoate 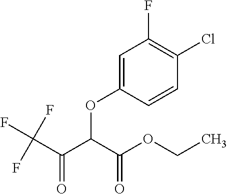 [SMILES: CCOC(=O)C(Oc1ccc(Cl)c(F)c1)C(=O)C(F)(F)F] (66% of theory) | LC-MS (Method 1): Rₜ = 1.01 min; MS (ESneg): m/z = 326.9 (M − H)⁻ |
| 11A | ethyl 2-(3-chloro-4-fluorophenoxy)-4,4,4- trifluoro-3-oxobutanoate 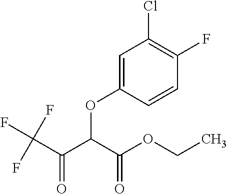 [SMILES: CCOC(=O)C(Oc1ccc(F)c(Cl)c1)C(=O)C(F)(F)F] (74% of theory) | LC-MS (Method 1): Rₜ = 1.00 min; MS (ESneg): m/z = 326.9 (M − H)⁻ |
| 12A | ethyl 4,4,4-trifluoro-2-[4-fluoro-3- (trifluoromethyl)phenoxy]-3-oxobutanoate 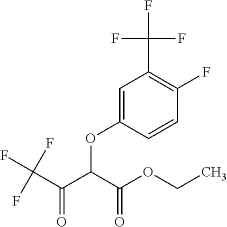 [SMILES: CCOC(=O)C(Oc1ccc(F)c(C(F)(F)F)c1)C(=O)C(F)(F)F] (61% of theory; 16 h) | LC-MS (Method 3): Rₜ = 2.35 min; MS (ESneg): m/z = 361.0 (M − H)⁻ |
| 13A | ethyl 2-(3-chlorophenoxy)-4,4,4-trifluoro-3- oxobutanoate 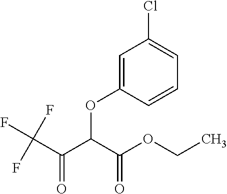 [SMILES: CCOC(=O)C(Oc1cccc(Cl)c1)C(=O)C(F)(F)F] (48% of theory; 3 h) | LC-MS (Method 1): Rₜ = 0.98-1.00 min; MS (ESneg): m/z = 309.0 (M − H)⁻ |
| 14A | ethyl 2-[(5-chloropyridin-3-yl)oxy]-4,4,4- trifluoro-3-oxobutanoate 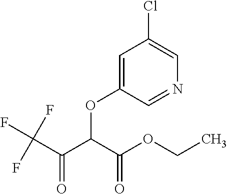 [SMILES: CCOC(=O)C(Oc1cncc(Cl)c1)C(=O)C(F)(F)F] (17% of theory; 16 h) | LC-MS (Method 1): Rₜ = 0.83-0.86 min; MS (ESneg): m/z = 309.9 (M − H)⁻ |
| 15A | ethyl 2-(3,4-difluorophenoxy)-4,4,4-trifluoro- 3-oxobutanoate 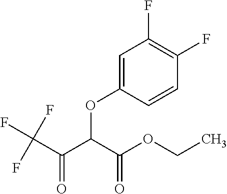 [SMILES: CCOC(=O)C(Oc1ccc(F)c(F)c1)C(=O)C(F)(F)F] (66% of theory; 3 h) | LC-MS (Method 1): Rₜ = 0.95 min; MS (ESneg): m/z = 311.0 (M − H)⁻ |
| 16A | ethyl 2-(3,4-dichlorophenoxy)-4,4,4-trifluoro- 3-oxobutanoate 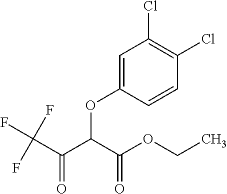 [SMILES: CCOC(=O)C(Oc1ccc(Cl)c(Cl)c1)C(=O)C(F)(F)F] (89% of theory; 3 h) | LC-MS (Method 3): Rₜ = 2.31 min; MS (ESneg): m/z = 343.0 (M − H)⁻ |

### Example 17A

### Ethyl 4,4,4-trifluoro-3-oxo-2-[3-(trifluoromethyl)benzyl]butanoate

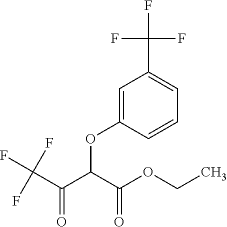 [SMILES: CCOC(=O)C(Oc1cccc(C(F)(F)F)c1)C(=O)C(F)(F)F]

10.8 g (83.7 mmol) of N,N-diisopropylethylamine and 1.77 g (41.8 mmol) of lithium chloride were added to 10 g (41.8 mmol) of 3-(bromomethyl)benzotrifluoride and 11.6 g (62.75 mmol) of ethyl trifluoroacetate in 51.6 ml of THF. The mixture was stirred at 67° C. for 18 h. The reaction was then concentrated under reduced pressure and the residue was taken up in ethyl acetate. The solution was washed with 1 N hydrochloric acid and the organic phase was dried over sodium sulphate, filtered and concentrated. The yellow oil (9.56 g, 27% of theory), which was obtained in a purity of 40% (HPLC), was used without further purification for the next step.

LC-MS (Method 1): Rₜ=1.12 min; MS (ESneg): m/z=341 (M−H)⁻.

Analogously to Example 17A, the following compound was prepared from the corresponding benzyl halide:

TABLE 3

| Example | IUPAC name/structure | |
|---|---|---|
| No. | (yield) | Analytical data |
| 18A | ethyl 2-(3-chlorobenzyl)-4,4,4-trifluoro-3- oxobutanoate 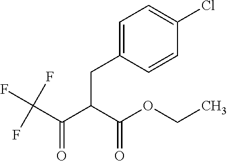 [SMILES: CCOC(=O)C(Cc1ccc(Cl)cc1)C(=O)C(F)(F)F] (42% of theory) | LC-MS (Method 1): Rₜ = 1.09 min; MS (ESneg): m/z = 307.1 (M − H)⁻ |

The following synthesis intermediates were prepared analogously to the method described in WO 2011/114148 (Methode XX) from the corresponding benzyl halides:

TABLE 4

| Example | IUPAC name/structure | |
|---|---|---|
| No. | (yield) | Analytical data |
| 19A | ethyl 4,4,4-trifluoro-2-[3-fluoro-5- (trifluoromethyl)benzyl]-3-oxobutanoate 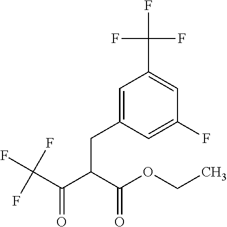 [SMILES: CCOC(=O)C(Cc1cc(F)cc(C(F)(F)F)c1)C(=O)C(F)(F)F] (62% of theory) | LC-MS (Method 1): Rₜ = 1.10 min and 1.37 min; MS (ESneg): m/z = 359.1 (M − H)⁻ |
| 20A | ethyl 2-(4-chloro-3-fluorobenzyl)-4,4,4- trifluoro-3-oxobutanoate 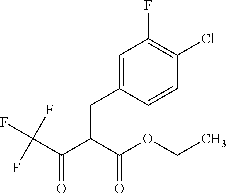 [SMILES: CCOC(=O)C(Cc1ccc(Cl)c(F)c1)C(=O)C(F)(F)F] (43% of theory) | LC-MS (Method 1): Rₜ = 1.07 min; MS (ESneg): m/z = 325.0 (M − H)⁻ |
| 21A | ethyl 2-[4-chloro-3-(trifluoromethyl)benzyl]- 4,4,4-trifluoro-3-oxobutanoate 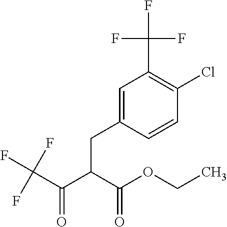 [SMILES: CCOC(=O)C(Cc1ccc(Cl)c(C(F)(F)F)c1)C(=O)C(F)(F)F] (44% of theory) | LC-MS (Method 1): Rₜ = 1.14 min; MS (ESneg): m/z = 374.9 (M − H)⁻ |
| 22A | ethyl 2-(3-chloro-4-methylbenzyl)-4,4,4- trifluoro-3-oxobutanoate 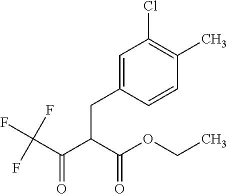 [SMILES: CCOC(=O)C(Cc1ccc(C)c(Cl)c1)C(=O)C(F)(F)F] (34% of theory) | LC-MS (Method 1): Rₜ = 1.12 min; MS (ESneg): m/z = 321.1 (M − H)⁻ |
| 23A | ethyl 2-(3-chloro-4-fluorobenzyl)-4,4,4- trifluoro-3-oxobutanoate 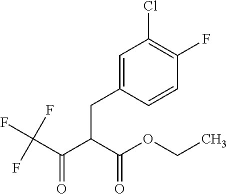 [SMILES: CCOC(=O)C(Cc1ccc(F)c(Cl)c1)C(=O)C(F)(F)F] (38% of theory) | LC-MS (Method 1): Rₜ = 1.06 min; MS (ESneg): m/z = 325.1 (M − H)⁻ |
| 24A | ethyl 2-[3-chloro-4-(trifluoromethyl)benzyl]- 4,4,4-trifluoro-3-oxobutanoate 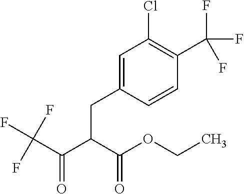 [SMILES: CCOC(=O)C(Cc1ccc(C(F)(F)F)c(Cl)c1)C(=O)C(F)(F)F] (27% of theory) | LC-MS (Method 1): Rₜ = 1.14 min; MS (ESneg): m/z = 375.1 (M − H)⁻ |

### Example 25A

### Methyl [5-(3,4-dichlorobenzyl)-6-oxo-4-(trifluoromethyl)-1,6-dihydropyrimidin-2-yl]acetate

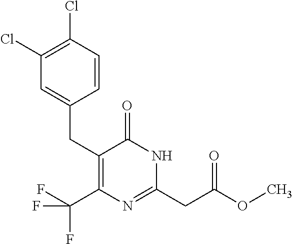 [SMILES: COC(=O)Cc1nc(C(F)(F)F)c(Cc2ccc(Cl)c(Cl)c2)c(=O)[nH]1]

Under argon and at 23° C., 1.13 g (20.89 mmol) of sodium methoxide were added to a solution of 3 g (19.66 mmol) of methyl 3-amino-3-iminopropanoate hydrochloride in 5 ml of methanol. The mixture was stirred at 23° C. for 15 min, and 0.84 g (2.46 mmol) of ethyl 2-(3,4-dichlorobenzyl)-4,4,4-trifluoro-3-oxobutanoate [CAS 179110-12-4; WO 2012/041817, Intermediate 56], dissolved in 5 ml of methanol, was then added. The mixture was stirred initially at 23° C. for 30 min and then under reflux for 16 h. The mixture was then applied to kieselguhr and purified directly by flash chromatography (40 g of silica gel, mobile phase cyclohexane/ethyl acetate). This gave 302 mg (26% of theory; purity 84%) of the title compound.

LC-MS (Method 1): Rₜ=1.13 min; MS (ESpos): m/z=395.0 (M+H)⁺

¹H NMR (400 MHz, DMSO-d₆): δ=3.67 (s, 3H), 3.79 (s, 2H), 3.92 (s, 2H), 7.13 (dd, 1H), 7.43 (d, 1H), 7.54 (d, 1H), 13.32 (br. s, 1H).

The following compounds are known from the literature, commercially available or can be prepared analogously to Example 2A:

TABLE 5

| Example | | |
|---|---|---|
| No. | IUPAC name/structure | Analytical data or CAS number |
| 26A | ethyl [4-chloro-3- (trifluoromethoxy)phenoxy]acetate 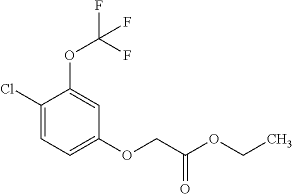 [SMILES: CCOC(=O)COc1ccc(Cl)c(OC(F)(F)F)c1] | LC-MS (Method 1): Rₜ = 1.18 min; MS (ESneg): m/z = 297.1 (M − H)⁻ |
| 27A | ethyl (3-chloro-4-methylphenoxy)acetate 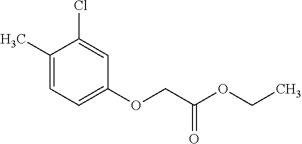 | LC-MS (Method 1): Rₜ = 2.38 min; MS (ESpos): m/z = 229.2 (M + H)⁺ |
| 28A | ethyl (4-chloro-3-methylphenoxy)acetate 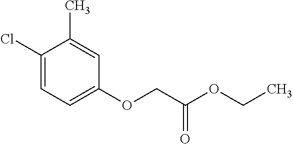 | CAS 30406-61-2 |
| 29A | ethyl (4-chlorophenoxy)acetate 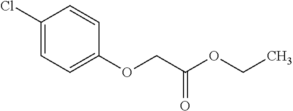 | CAS 14426-42-7 |
| 30A | ethyl [4-(trifluoromethyl)phenoxy]acetate 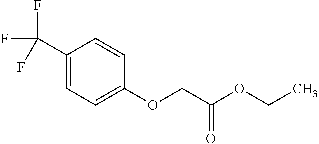 [SMILES: CCOC(=O)COc1ccc(C(F)(F)F)cc1] | CAS 442125-30-6 |
| 31A | ethyl [3-(trifluoromethyl)phenoxy]acetate 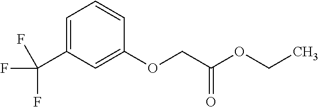 [SMILES: CCOC(=O)COc1cccc(C(F)(F)F)c1] | CAS 22897-99-0 |

The following synthesis intermediates were prepared analogously to Example 9A:

TABLE 6

| Example | IUPAC name/structure | |
|---|---|---|
| No. | (yield; reaction time) | Analytical data |
| 32A | ethyl 2-[4-chloro-3- (trifluoromethoxy)phenoxy]-4,4,4-trifluoro-3- oxobutanoate 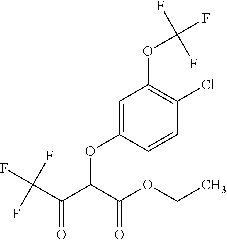 [SMILES: CCOC(=O)C(Oc1ccc(Cl)c(OC(F)(F)F)c1)C(=O)C(F)(F)F] (94% of theory; 3 h) | LC-MS (Method 1): Rₜ = 1.07 min; MS (ESneg): m/z = 393.0 (M − H)⁻ |
| 33A | ethyl 2-(3-chloro-4-methylphenoxy)-4,4,4- trifluoro-3-oxobutanoate 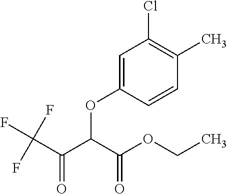 [SMILES: CCOC(=O)C(Oc1ccc(C)c(Cl)c1)C(=O)C(F)(F)F] (29% of theory; 16 h) | LC-MS (Method 3): Rₜ = 2.28 min; MS (ESneg): m/z = 323.0 (M − H)⁻ |
| 34A | ethyl 2-(4-chloro-3-methylphenoxy)-4,4,4- trifluoro-3-oxobutanoate 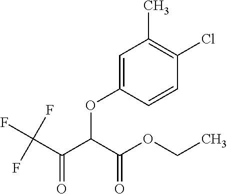 [SMILES: CCOC(=O)C(Oc1ccc(Cl)c(C)c1)C(=O)C(F)(F)F] (37% of theory; 16 h) | LC-MS (Method 3): Rₜ = 2.28 min; MS (ESneg): m/z = 323.0 (M − H)⁻ |
| 35A | ethyl 2-[4-chlorophenoxy]-4,4,4-trifluoro-3- oxobutanoate 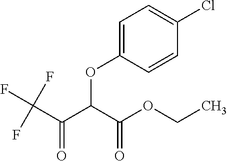 [SMILES: CCOC(=O)C(Oc1ccc(Cl)cc1)C(=O)C(F)(F)F] (78% of theory) | LC-MS (Method 1): Rₜ = 0.93 min; MS (ESneg): m/z = 309 (M − H)⁻ |
| 36A | ethyl 4,4,4-trifluoro-3-oxo-2-[4- (trifluoromethyl)phenoxy]butanoate 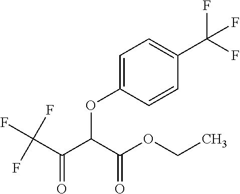 [SMILES: CCOC(=O)C(Oc1ccc(C(F)(F)F)cc1)C(=O)C(F)(F)F] (42% of theory) | LC-MS (Method 3): Rₜ = 2.24 min; MS (ESneg): m/z = 343.0 (M − H)⁻ |
| 37A | ethyl 4,4,4-trifluoro-3-oxo-2-[3- (trifluoromethyl)phenoxy]butanoate 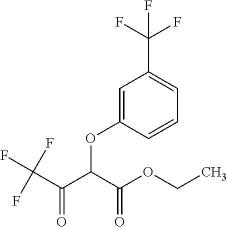 [SMILES: CCOC(=O)C(Oc1cccc(C(F)(F)F)c1)C(=O)C(F)(F)F] (55% of theory) | ¹H NMR (400 MHz, DMSO-d₆): δ = 1.12-1.18 (m, 3H), 4.11-4.23 (m, 2H), 4.98 (s, 1H), 7.15-7.23 (m, 2H), 7.37 (dd, 1H), 7.53-7.59 (m, 1H). |

The following synthesis intermediates were prepared analogously to the method described in WO 2011/114148 (Method XX) from the corresponding pyridylmethyl halides:

TABLE 7

| Example | IUPAC name/structure | |
|---|---|---|
| No. | (yield) | Analytical data |
| 38A | ethyl 2-[(6-chloropyridin-3-yl)methyl]-4,4,4- trifluoro-3-oxobutanoate 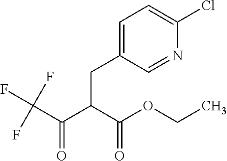 [SMILES: CCOC(=O)C(Cc1ccc(Cl)nc1)C(=O)C(F)(F)F] (51% of theory) | LC-MS (Method 3): Rₜ = 1.91 min; MS (ESneg): m/z = 308.2 (M − H)⁻ |
| 39A | ethyl 2-[(5,6-dichloropyridin-3-yl)methyl]- 4,4,4-trifluoro-3-oxobutanoate 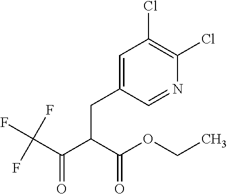 [SMILES: CCOC(=O)C(Cc1cnc(Cl)c(Cl)c1)C(=O)C(F)(F)F] (57% of theory) | LC-MS (Method 3): Rₜ = 2.15 min; MS (ESneg): m/z = 342.1 (M − H)⁻ |

### Example 40A

### 5-(3,4-Dichlorophenoxy)-2-(methylsulphanyl)-6-(trifluoromethyl)pyrimidin-4(3H)-one

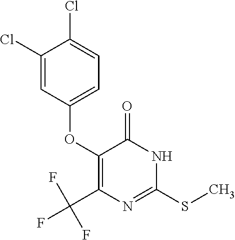 [SMILES: CSc1nc(C(F)(F)F)c(Oc2ccc(Cl)c(Cl)c2)c(=O)[nH]1]

A mixture of 8.65 g (63 mmol) of potassium carbonate, 6.77 g (75 mmol) of S-methylisothiourea hemisulphate and 8 g (12.5 mmol; purity 54%) of ethyl 2-(3,4-dichlorophenoxy)-4,4,4-trifluoro-3-oxobutanoate in 101 ml of dioxane was stirred at 95° C. for 2 h. 1 ml of 1 N hydrochloric acid was then added, the mixture was concentrated under reduced pressure and 300 ml of water were added to the residue. The precipitated solid was filtered off with suction and washed successively with water, petroleum ether and diethyl ether. Drying under high vacuum gave 5.85 g (91% of theory) of the title compound in a purity of 72% (HPLC).

LC-MS (Method 1): Rₜ=1.13 min; MS (ESpos): m/z=371.0 (M+H)⁺

¹H NMR (400 MHz, DMSO-d₆): δ=2.56 (s, 3H), 7.13 (dd, 1H), 7.48 (d, 1H), 7.56 (d, 1H), 13.72 (br. s, 1H).

### Example 41A

### 5-(3,4-Dichlorophenoxy)-2-(methylsulphonyl)-6-(trifluoromethyl)pyrimidin-4(3H)-one

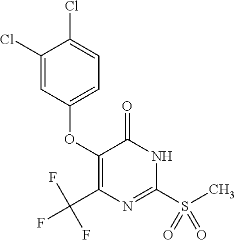 [SMILES: CS(=O)(=O)c1nc(C(F)(F)F)c(Oc2ccc(Cl)c(Cl)c2)c(=O)[nH]1]

A mixture of 4 g (7.8 mmol) of 5-(3,4-dichlorophenoxy)-2-(methylsulphanyl)-6-(trifluoromethyl)pyrimidin-4(3H)-one (purity 72%), 14.37 g (23.4 mmol) of Oxone™ and 4.07 g (23.4 mmol) of dipotassium phosphate was stirred in 68 ml of dioxane and 32 ml of water at 22° C. for 18 h. The reaction mixture was subsequently stirred with 1 litre of water and the resulting white crystals were filtered off with suction. After washing with 100 ml of water and 50 ml of petroleum ether, the solid was dried under high vacuum. This gave 2.46 g (75% of theory) of the title compound.

LC-MS (Method 1): Rₜ=0.95 min; MS (ESneg): m/z=400.9 (M−H)⁻.

### Example 42A

### Ethyl 2-[1-(3,4-dichlorophenyl)ethyl]-4,4,4-trifluoro-3-oxobutanoate

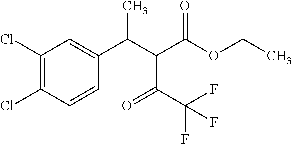 [SMILES: CCOC(=O)C(C(=O)C(F)(F)F)C(C)c1ccc(Cl)c(Cl)c1]

95 mg (0.5 mmol) of copper(I) iodide were suspended in 5 ml of THF and the mixture was cooled to −78° C. At this temperature, 0.33 ml (1.0 mmol) of methylmagnesium bromide (3 M solution in diethyl ether) and 0.21 ml (1.0 mmol) of trimethylsilyl chloride were added dropwise and the mixture was stirred at −78° C. for another 10 min. 123 mg (0.5 mmol) of ethyl (2E)-3-(3,4-dichlorophenyl)acrylate [lit. e.g.: Y. Liu and J. Zhou, Chem. Commun. 49 (39), 4421-4423 (2013)], dissolved in 5 ml of THF, were then added dropwise. The reaction mixture was warmed to RT over a period of 4 h and then once more cooled to −78° C. 0.2 ml (1.5 mmol) of trifluoroacetic anhydride were added and the reaction mixture was then stirred at RT for 1 h. A 1:1 mixture of saturated aqueous ammonium chloride solution and 1 N hydrochloric acid was then added to the reaction mixture, and the reaction mixture was extracted three times with ethyl acetate. The combined organic phases were washed with a 1:1 mixture of saturated aqueous ammonium chloride solution and 25% strength aqueous ammonia until the colour of the aqueous phase was no longer blue and the organic phase was colourless. The organic phase was washed with saturated aqueous sodium chloride solution and dried over sodium sulphate. After removal of the drying agent by filtration, the mixture was concentrated under reduced pressure. Drying of the residue under high vacuum thus gave 178 mg (85% of theory, purity 85%) of the title compound. The product was able to be employed for further reactions without further purification.

LC-MS (Method 6): Rₜ=1.46 min, MS (ESneg): m/z=355.0 (M−H)⁻; Rₜ=1.50 min, MS (ESneg): m/z=355.0 (M−H)⁻; Rₜ=1.66 min, MS (ESneg): m/z=355.0 (M−H)⁻ [mixture of diastereomers and keto-enol tautomers].

### WORKING EXAMPLES

### Example 1

### 2-Amino-5-(3,4-dichlorobenzyl)-6-(trifluoromethyl)pyrimidin-4(3H)-one

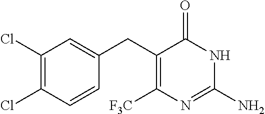 [SMILES: Nc1nc(C(F)(F)F)c(Cc2ccc(Cl)c(Cl)c2)c(=O)[nH]1]

A mixture of 110 mg (0.8 mmol) of potassium carbonate, 76 mg (0.8 mmol) of guanidine hydrochloride and 400 mg (0.8 mmol) of ethyl 2-(3,4-dichlorobenzyl)-4,4,4-trifluoro-3-oxobutanoate (purity 68%; CAS 179110-12-4; WO 2012/041817, Intermediate 56) in 4 ml of ethanol was heated under reflux for 6 h. The solution was then concentrated under reduced pressure and the residue was purified by preparative HPLC (mobile phase: acetonitrile/water gradient with 0.1% of formic acid). This gave 78 mg (29% of theory) of the title compound.

LC-MS (Method 1): Rₜ=1.04 min; MS (ESpos): m/z=338.1 (M+H)⁺

¹H NMR (400 MHz, DMSO-d₆): δ=3.74 (s, 2H), 6.98 (br. s, 2H), 7.11 (dd, 1H), 7.38 (d, 1H), 7.51 (d, 1H), 11.53 (br. s, 1H).

### Example 2

### 2-Amino-5-[4-chloro-3-(trifluoromethyl)phenoxy]-6-(trifluoromethyl)pyrimidin-4(3H)-one

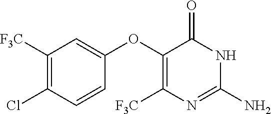 [SMILES: Nc1nc(C(F)(F)F)c(Oc2ccc(Cl)c(C(F)(F)F)c2)c(=O)[nH]1]

A mixture of 20.15 g (146 mmol) of potassium carbonate, 10.5 g (109 mmol) of guanidine hydrochloride and 20 g (36.5 mmol, purity 69%) of ethyl 2-[4-chloro-3-(trifluoromethyl)phenoxy]-4,4,4-trifluoro-3-oxobutanoate (Example 9A) in 150 ml of dioxane was heated under reflux for 1 h. The reaction mixture was then added to 1.8 litres of water and neutralized with 1 N hydrochloric acid. The precipitated solid was filtered off with suction, washed with water and taken up in a little ethyl acetate, and the resulting solution was added dropwise with stirring to 1 litre of petroleum ether. The resulting precipitate was filtered off with suction, taken up in 100 ml of 0.5 N sulphuric acid and 100 ml of acetonitrile, stirred for 30 min and then added to 1 litre of water. After 15 min of stirring, the mixture was once more filtered off with suction and the precipitate was washed with water. The product was taken up in ethyl acetate and, together with silica gel, reconcentrated under reduced pressure. This material was chromatographed on silica gel using a mixture of cyclohexane and ethyl acetate (1:1). The product-containing fractions were concentrated and the residue was dried under reduced pressure. This gave 10.5 g (77% of theory) of the title compound in a purity of 99% (HPLC).

LC-MS (Method 1): Rₜ=1.02 min; MS (ESpos): m/z=374.0 (M+H)⁺

¹H NMR (400 MHz, DMSO-d₆): δ=7.07 (br. s, 2H), 7.31 (dd, 1H), 7.42 (d, 1H), 7.62 (d, 1H), 11.86 (br. s, 1H).

### Example 3

### 2-Amino-5-(3,4-dichlorophenoxy)-6-(trifluoromethyl)pyrimidin-4(3H)-one

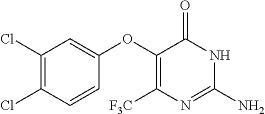 [SMILES: Nc1nc(C(F)(F)F)c(Oc2ccc(Cl)c(Cl)c2)c(=O)[nH]1]

A mixture of 5.53 g (40 mmol) of potassium carbonate, 2.87 g (30 mmol) of guanidine hydrochloride and 6.70 g (10 mmol, purity 52%) of ethyl 2-(3,4-dichlorophenoxy)-4,4,4-trifluoro-3-oxobutanoate (Example 16A) in 33 ml of dioxane was stirred at 90° C. for 1 h. The reaction mixture was then added to 0.8 litre of water and neutralized with 1 N hydrochloric acid. The precipitated solid was filtered off with suction and washed with 100 ml of water and 200 ml of petroleum ether. The residue was chromatographed on silica gel using a mixture of cyclohexane and ethyl acetate (initially 1:1, then 0:1). The product-containing fractions were concentrated and the residue was dried under reduced pressure. This gave 3.04 g (87% of theory) of the title compound in a purity of 97% (HPLC).

LC-MS (Method 1): Rₜ=0.99 min; MS (ESpos): m/z=340.0 (M+H)⁺

¹H NMR (400 MHz, DMSO-d₆): δ=7.00-7.18 (br. s, 2H), 7.01 (dd, 1H), 7.33 (d, 1H), 7.52 (d, 1H), 11.80 (br. s, 1H).

The exemplary compounds listed in Table 8 were prepared analogously to Example 1 by reacting guanidine hydrochloride with the appropriate benzyl- or phenoxy-substituted trifluoromethyl keto esters:

TABLE 8

| Example | IUPAC name/structure | |
|---|---|---|
| No. | (yield, reaction conditions) | Analytical data |
| 4 | 2-amino-5-(3-chloro-4-fluorophenoxy)-6- (trifluoromethyl)pyrimidin-4(3H)-one 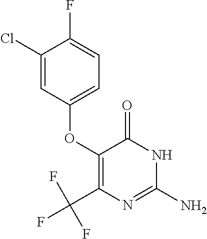 [SMILES: Nc1nc(C(F)(F)F)c(Oc2ccc(F)c(Cl)c2)c(=O)[nH]1] (85% of theory; reaction time: 18 h; solvent: dioxane; 5 eq. of potassium carbonate) | LC-MS (Method 1): Rₜ = 0.94 min; MS (ESpos): m/z = 324 (M + H)⁺ ¹H NMR (400 MHz, DMSO-d₆): δ = 6.88-7.14 (m, 3H), 7.25 (dd, 1H), 7.32 (t, 1H), 11.79 (br. s, 1H). |
| 5 | 2-amino-5-(4-chloro-3-fluorophenoxy)-6- (trifluoromethyl)pyrimidin-4(3H)-one 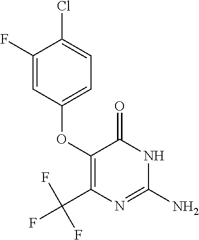 [SMILES: Nc1nc(C(F)(F)F)c(Oc2ccc(Cl)c(F)c2)c(=O)[nH]1] (80% of theory; reaction time: 18 h; solvent: dioxane; 5 eq. of potassium carbonate) | LC-MS (Method 1): Rₜ = 0.96 min; MS (ESpos): m/z = 324 (M + H)⁺ ¹H NMR (400 MHz, DMSO-d₆): δ = 6.87 (dd, 1H), 7.03 (br. s, 2H), 7.18 (dd, 1H), 7.47 (t, 1H), 11.82 (br. s, 1H). |
| 6 | 2-amino-5-[4-fluoro-3- (trifluoromethyl)phenoxy]-6- (trifluoromethyl)pyrimidin-4(3H)-one 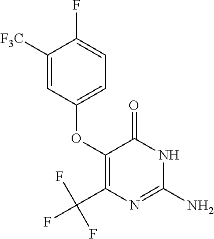 [SMILES: Nc1nc(C(F)(F)F)c(Oc2ccc(F)c(C(F)(F)F)c2)c(=O)[nH]1] (62% of theory; reaction time: 16 h; solvent: dioxane, 4 eq. of potassium carbonate) | LC-MS (Method 1): Rₜ = 0.98 min; MS (ESpos): m/z = 358.1 (M + H)⁺ ¹H NMR (400 MHz, DMSO-d₆): δ = 7.05 (br. s, 2H), 7.30-7.39 (m, 2H), 7.39-7.48 (m, 1H), 11.81 (br. s, 1H). |
| 7 | 2-amino-5-(3-chloro-4-fluorobenzyl)-6- (trifluoromethyl)pyrimidin-4(3H)-one 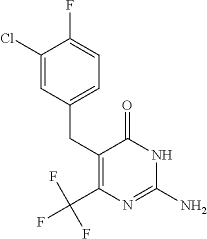 [SMILES: Nc1nc(C(F)(F)F)c(Cc2ccc(F)c(Cl)c2)c(=O)[nH]1] (19% of theory; reaction time: 18 h) | LC-MS (Method 1): Rₜ = 0.95 min; MS (ESpos): m/z = 322.2 (M + H)⁺ ¹H NMR (400 MHz, DMSO-d₆): δ = 3.74 (s, 2H), 6.80-7.38 (m, 5H), 11.52 (br. s, 1H). |
| 8 | 2-amino-5-[4-chloro-3-(trifluoromethyl)benzyl]- 6-(trifluoromethyl)pyrimidin-4(3H)-one 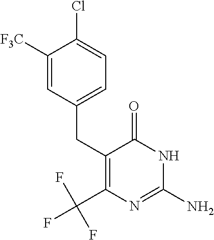 [SMILES: Nc1nc(C(F)(F)F)c(Cc2ccc(Cl)c(C(F)(F)F)c2)c(=O)[nH]1] (35% of theory; reaction time: 16 h; solvent: dioxane, 4 eq. of potassium carbonate) | LC-MS (Method 1): Rₜ = 1.05 min; MS (ESpos): m/z = 372.1 (M + H)⁺ ¹H NMR (400 MHz, DMSO-d₆): δ = 3.83 (s, 2H), 7.42 (dd, 1H), 7.63-7.65 (m, 1H), 11.56 (br. s, 1H). |
| 9 | 2-amino-5-(3-chlorophenoxy)-6- (trifluoromethyl)pyrimidin-4(3H)-one 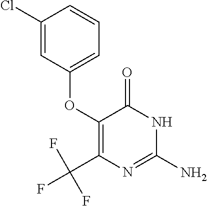 [SMILES: Nc1nc(C(F)(F)F)c(Oc2cccc(Cl)c2)c(=O)[nH]1] (99% of theory; reaction time: 18 h; solvent: dioxane; 5 eq. of potassium carbonate) | LC-MS (Method 1): Rₜ = 0.91 min; MS (ESpos): m/z = 306.1 (M + H)⁺ ¹H NMR (400 MHz, DMSO-d₆): δ = 6.93 (dd, 1H), 6.97-7.11 (m, 3H), 7.31 (t, 1H), 11.79 (br. s, 1H). |
| 10 | 2-amino-5-(3,4-difluorophenoxy)-6- (trifluoromethyl)pyrimidin-4(3H)-one 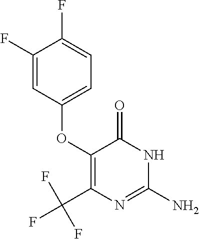 [SMILES: Nc1nc(C(F)(F)F)c(Oc2ccc(F)c(F)c2)c(=O)[nH]1] (73% of theory; reaction time: 18 h; solvent: dioxane; 5 eq. of potassium carbonate) | LC-MS (Method 1): Rₜ = 0.89 min; MS (ESpos): m/z = 308.1 (M + H)⁺ ¹H NMR (400 MHz, DMSO-d₆): δ = 6.77-6.84 (m, 1H), 6.88-7.12 (m, 2H), 7.28-7.40 (m, 1H), 11.79 (br. s, 1H). |
| 11 | 2-amino-5-(3-chloro-4-methylbenzyl)-6- (trifluoromethyl)pyrimidin-4(3H)-one 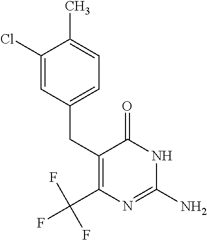 [SMILES: Cc1ccc(Cc2c(C(F)(F)F)nc(N)[nH]c2=O)cc1Cl] (9% of theory; reaction time: 18 h) | conditions of prep. HPLC purification: column: Daicel Chiracel OD-H 5 μm, 250 × 20 mm; flow rate: 20 ml/min; run time: 9 min; detection: 230 nm; mobile phase: isohexane/ethanol 80:20. LC-MS (Method 1): Rₜ = 1.02 min; MS (ESpos): m/z = 318.0 (M + H)⁺ ¹H NMR (400 MHz, DMSO-d₆): δ = 2.26 (s, 3H), 3.71 (br. s, 2H), 6.81- 7.34 (m, 5H), 11.49 (br. s, 1H). |
| 12 | 2-amino-5-[(5-chloropyridin-3-yl)oxy]-6- (trifluoromethyl)pyrimidin-4(3H)-one 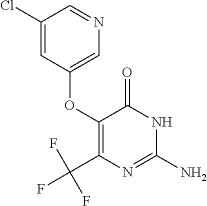 [SMILES: Nc1nc(C(F)(F)F)c(Oc2cncc(Cl)c2)c(=O)[nH]1] (3% of theory; reaction time: 16 h; solvent: dioxane; 4 eq. of potassium carbonate) | LC-MS (Method 1): Rₜ = 0.80 min; MS (ESpos): m/z = 349.0 (M + H)⁺ ¹H NMR (400 MHz, DMSO-d₆): δ = 7.08 (br. s, 2H), 7.72 (t, 2H), 8.31 (d, 1H), 8.36 (d, 1H), 11.90 (br. s, 1H). |
| 13 | 2-amino-5-(4-chloro-3-fluorobenzyl)-6- (trifluoromethyl)pyrimidin-4(3H)-one 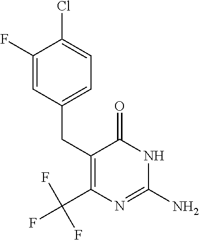 [SMILES: Nc1nc(C(F)(F)F)c(Cc2ccc(Cl)c(F)c2)c(=O)[nH]1] (45% of theory; reaction time: 16 h; solvent: dioxane; 4 eq. of potassium carbonate) | LC-MS (Method 1): Rₜ = 0.98 min; MS (ESpos): m/z = 322.1 (M + H)⁺ ¹H NMR (400 MHz, DMSO-d₆): δ = 3.76 (s, 2H), 6.80-7.10 (m, 3H), 7.16 (d, 1H), 7.45 (t, 1H), 11.50 (br. s, 1H). |
| 14 | 2-amino-5-[3-chloro-4-(trifluoromethyl)benzyl]- 6-(trifluoromethyl)pyrimidin-4(3H)-one 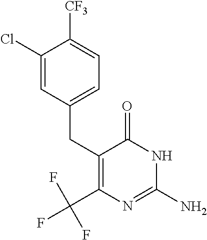 [SMILES: Nc1nc(C(F)(F)F)c(Cc2ccc(C(F)(F)F)c(Cl)c2)c(=O)[nH]1] (16% of theory; reaction time: 18 h) | LC-MS (Method 1): Rₜ = 1.05 min; MS (ESpos): m/z = 372.1 (M + H)⁺ ¹H NMR (400 MHz, DMSO-d₆): δ = 3.84 (br. s, 2H), 6.63-8.02 (m, 5H), 11.56 (br. s, 1H). |

### Example 15

### 2-Amino-5-[(3,4-dichlorophenyl)sulphanyl]-6-(trifluoromethyl)pyrimidin-4(3H)-one

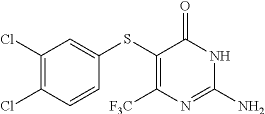 [SMILES: Nc1nc(C(F)(F)F)c(Sc2ccc(Cl)c(Cl)c2)c(=O)[nH]1]

A mixture of 258 mg (1 mmol) of 2-amino-5-bromo-6-(trifluoromethyl)pyrimidin-4(3H)-one [CAS 1583-00-2; preparation analogously to WO 2011/114148, Method XIX], 326 mg (1 mmol) of caesium carbonate and 179 mg (1 mmol) of 3,4-dichlorothiophenol in 5 ml of ethylene glycol was stirred at 110° C. for 6 h. The mixture was then concentrated. The residue was purified by preparative HPLC (mobile phase: acetonitrile/water gradient with 0.1% of formic acid). The product-containing fractions were concentrated and the residue was dried under reduced pressure. This gave 81 mg (23% of theory) of the title compound in a purity of 100% (HPLC).

LC-MS (Method 1): Rₜ=1.01 min; MS (ESpos): m/z=356.0 (M+H)⁺

¹H NMR (400 MHz, DMSO-d₆): δ=6.15-8.95 (br. s, 2H), 7.09 (dd, 1H), 7.36 (d, 1H), 7.49 (d, 1H), 11.80 (br. s, 1H).

The following exemplary compounds were prepared in an analogous manner:

TABLE 9

| Example | IUPAC name/structure | |
|---|---|---|
| No. | (yield, reaction conditions) | Analytical data |
| 16 | 2-amino-5-[(4-chlorophenyl)sulphanyl]-6- (trifluoromethyl)pyrimidin-4(3H)-one 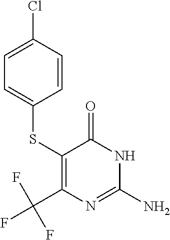 [SMILES: Nc1nc(C(F)(F)F)c(Sc2ccc(Cl)cc2)c(=O)[nH]1] (27% of theory; reaction time: 6 h, 150° C.; solvent: ethylene glycol; 3 eq. of 4-chlorothiophenol, 1 eq. caseium carbonate) | LC-MS (Method 1): Rₜ = 0.95 min; MS (ESpos): m/z = 322.1 (M + H)⁺ ¹H NMR (400 MHz, DMSO-d₆): δ = 6.56-8.62 (br. s, 2H), 7.12 (d, 2H), 7.31 (d, 2H), 11.76 (br. s, 1H). |
| 17 | 2-amino-5-{[4-chloro-3- (trifluoromethyl)phenyl]sulphanyl}-6- (trifluoromethyl)pyrimidin-4(3H)-one 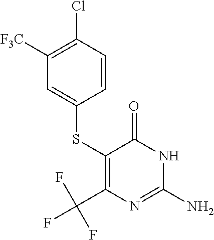 [SMILES: Nc1nc(C(F)(F)F)c(Sc2ccc(Cl)c(C(F)(F)F)c2)c(=O)[nH]1] (7% of theory; reaction time: 24 h, 150° C.; solvent: ethylene glycol; 3 eq. of 4-chloro-3- (trifluoromethyl)thiophenol, 1 eq. caseium carbonate) | LC-MS (Method 1): Rₜ = 1.06 min; MS (ESpos): m/z = 390.0 (M + H)⁺ ¹H NMR (400 MHz, DMSO-d₆): δ = 6.35-8.72 (br. s, 2H), 7.38 (dd, 1H), 7.55-7.62 (m, 2H), 11.85 (br. s, 1H). |

### Example 18

### 5-(3,4-Dichlorobenzyl)-2-methyl-6-(trifluoromethyl)pyrimidin-4(3H)-one

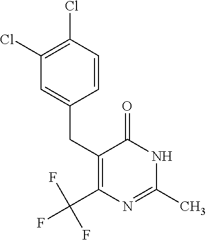 [SMILES: Cc1nc(C(F)(F)F)c(Cc2ccc(Cl)c(Cl)c2)c(=O)[nH]1]

175 mg (0.43 mmol) of methyl [5-(3,4-dichlorobenzyl)-6-oxo-4-(trifluoromethyl)-1,6-dihydropyrimidin-2-yl]acetate (Example 25A) were dissolved in 1.7 ml of THF, 1.72 ml of 1 N aqueous lithium hydroxide solution were added and the mixture was stirred at 23° C. for 18 h. The mixture was then neutralized with 1 N hydrochloric acid and purified directly by preparative HPLC [column: Chromatorex C18 10 μm, 250×30 mm; flow rate: 50 ml/min; run time: 45 min; detection: 210 nm; injection after 3 min of run time; mobile phase A: acetonitrile, mobile phase B: 0.1% aq. formic acid; gradient: 10% A (5.00 min)→95% A (35.00-40.00 min)→10% A (40.50-45.00 min)] Yield: 41% of theory.

LC-MS (Method 1): Rₜ=1.08 min; MS (ESpos): m/z=337.1 (M+H)⁺

¹H NMR (400 MHz, DMSO-d₆): δ=2.35 (s, 3H), 3.89 (s, 2H), 7.08-7.16 (m, 1H), 7.41-7.44 (m, 1H), 7.53 (d, 1H), 12.99-13.26 (m, 1H).

### Example 19

### 5-[3-Chloro-4-(trifluoromethyl)benzyl]-2-ethyl-6-(trifluoromethyl)pyrimidin-4(3H)-one

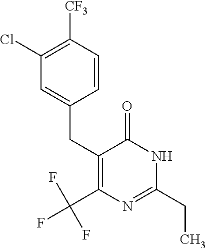 [SMILES: CCc1nc(C(F)(F)F)c(Cc2ccc(C(F)(F)F)c(Cl)c2)c(=O)[nH]1]

A mixture of 293 mg (2.1 mmol) of potassium carbonate, 172 mg (1.6 mmol) of propanimidamide hydrochloride and 200 mg (0.5 mmol) of ethyl 2-[3-chloro-4-(trifluoromethyl)benzyl]-4,4,4-trifluoro-3-oxobutanoate (Example 24A) in 2.3 ml of dioxane was heated under reflux for 18 h. The mixture was then filtered, the residue was washed with dioxane and the filtrate was purified by preparative HPLC (mobile phase: acetonitrile/water gradient with 0.1% of formic acid). This gave, from two reactions with, in total, 0.66 mmol of ethyl 2-[3-chloro-4-(trifluoromethyl)benzyl]-4,4,4-trifluoro-3-oxobutanoate, 48 mg (18% of theory) of the title compound.

LC-MS (Method 1): Rₜ=1.21 min; MS (ESpos): m/z=385.1 (M+H)⁺

¹H NMR (400 MHz, DMSO-d₆): δ=1.20 (t, J=7.5 Hz, 3H), 2.63 (q, J=7.5 Hz, 2H), 3.99 (s, 2H), 7.30 (d, J=8.1 Hz, 1H), 7.54 (s, 1H), 7.76 (d, J=8.2 Hz, 1H), 13.13 (br. s, 1H).

The exemplary compounds listed in Table 10 were prepared analogously to Example 19 or Example 25A by reacting the appropriate amidines (imidamides) or their salts with the appropriate benzyl- or phenoxy-substituted trifluoromethyl keto esters:

TABLE 10

| Example | IUPAC name/structure | |
|---|---|---|
| No. | (yield, reaction conditions) | Analytical data |
| 20 | 2-ethyl-6-(trifluoromethyl)-5-[3- (trifluoromethyl)benzyl]pyrimidin-4(3H)-one 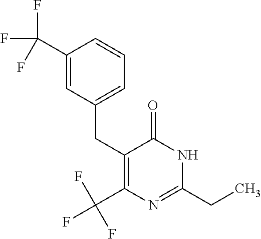 [SMILES: CCc1nc(C(F)(F)F)c(Cc2cccc(C(F)(F)F)c2)c(=O)[nH]1] (30% of theory; preparation analogous to Example 25A; base: sodium methoxide; solvent: methanol; reaction time: 10 h, 64° C.) | LC-MS (Method 1): Rₜ = 1.13 min; MS (ESpos): m/z = 351 (M + H)⁺ ¹H NMR (400 MHz, DMSO-d₆): δ = 1.20 (t, 3H), 2.62 (q, 2H), 4.00 (s, 2H), 7.41-7.46 (m, 1H), 7.48-7.54 (m, 1H), 7.55 (s, 2H), 12.88-13.20 (m, 1H). |
| 21 | 5-(3-chlorobenzyl)-2-cyclopropyl-6- (trifluoromethyl)pyrimidin-4(3H)-one 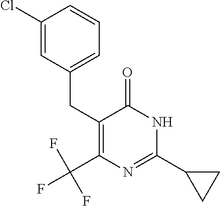 [SMILES: O=c1[nH]c(C2CC2)nc(C(F)(F)F)c1Cc1cccc(Cl)c1] (62% of theory; preparation analogous to Example 25A from ethyl 2-(3-chlorobenzyl)- 4,4,4-trifluoro-3-oxobutanoate (WO 2011/114148, Method XX); base: sodium methoxide; solvent: methanol; reaction time: 18 H, 64° C.) | LC-MS (Method 1): Rₜ = 1.19 min; MS (ESpos): m/z = 329 (M + H)⁺ ¹H NMR (400 MHz, DMSO-d₆): δ = 1.02-1.13 (m, 4H), 1.92-2.02 (m, 1H), 3.88 (s, 2H), 7.05-7.11 (m, 1H), 7.18-7.32 (m, 3H), 13.10-13.48 (m, 1H). |
| 22 | 2-butyl-5-(3,4-dichlorobenzyl)-6- (trifluoromethyl)pyrimidin-4(3H)-one 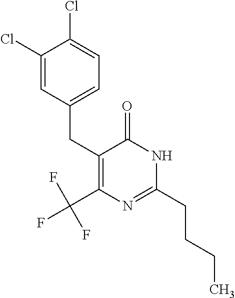 [SMILES: CCCCc1nc(C(F)(F)F)c(Cc2ccc(Cl)c(Cl)c2)c(=O)[nH]1] (82% of theory; reaction time: 16 h) | LC-MS (Method 1): Rₜ = 1.32 min; MS (ESpos): m/z = 379 (M + H)⁺ ¹H NMR (400 MHz, DMSO-d₆): δ = 0.90 (t, 3H), 1.29-1.38 (m, 2H), 1.61- 1.70 (m, 2H), 2.59 (t, 2H), 3.89 (s, 2H), 7.12 (dd, 1H), 7.43 (d, 1H), 7.53 (d, 1H), 13.02-13.15 (m, 1H). |
| 23 | 5-(4-chloro-3-fluorobenzyl)-2-cyclopropyl-6- (trifluoromethyl)pyrimidin-4(3H)-one 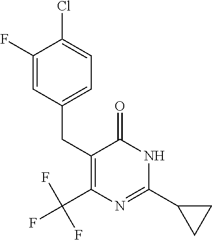 [SMILES: O=c1[nH]c(C2CC2)nc(C(F)(F)F)c1Cc1ccc(Cl)c(F)c1] (67% of theory; reaction time: 16 h) | LC-MS (Method 1): Rₜ = 1.19 min; MS (ESpos): m/z = 347 (M + H)⁺ ¹H NMR (400 MHz, DMSO-d₆): δ = 0.99-1.12 (m, 4H), 1.94-2.01 (m, 1H), 3.85-3.90 (m, 2H), 6.96-7.03 (m, 1H), 7.16-7.23 (m, 1H), 7.45 (t, 1H), 13.18-13.40 (m, 1H). |
| 24 | 5-[3-chloro-4-(trifluoromethyl)benzyl]-2- cyclopropyl-6-(trifluoromethyl)pyrimidin-4(3H)- one 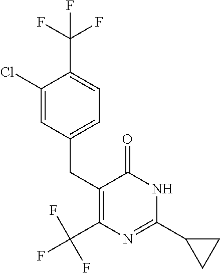 [SMILES: O=c1[nH]c(C2CC2)nc(C(F)(F)F)c1Cc1ccc(C(F)(F)F)c(Cl)c1] (32% of theory; 3 eq. of cyclopropane-1- carboximidamide hydrochloride; 4 eq. potassium carbonate; dioxane; reaction time: 18 h, reflux) | LC-MS (Method 1): Rₜ = 1.28 min; MS (ESpos): m/z = 397.2 (M + H)⁺ ¹H NMR (400 MHz, DMSO-d₆): δ = 0.99-1.21 (m, 4H), 1.91-2.10 (m, 1H), 3.96 (br. s, 2H), 7.29 (d, 1H), 7.54 (s, 1H), 7.75 (d, 1H), 13.35 (br. s, 1H). |
| 25 | 2-cyclopropyl-5-(3,4-dichlorophenoxy)-6- (trifluoromethyl)pyrimidin-4(3H)-one 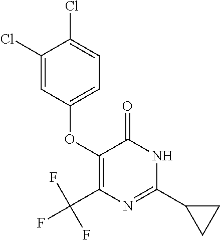 [SMILES: O=c1[nH]c(C2CC2)nc(C(F)(F)F)c1Oc1ccc(Cl)c(Cl)c1] (68% of theory; reaction time: 16 h) | LC-MS (Method 1): Rₜ = 1.20 min; MS (ESpos): m/z = 365 (M + H)⁺ ¹H NMR (400 MHz, DMSO-d₆): δ = 1.03-1.17 (m, 4H), 2.00 (d, 1H), 7.09 (dd, 1H), 7.43 (d, 1H), 7.55 (d, 1H), 13.58 (br. s, 1H). |
| 26 | 5-[4-chloro-3-(trifluoromethyl)benzyl]-2-ethyl-6- (trifluoromethyl)pyrimidin-4(3H)-one 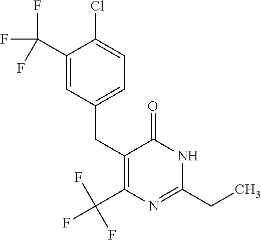 [SMILES: CCc1nc(C(F)(F)F)c(Cc2ccc(Cl)c(C(F)(F)F)c2)c(=O)[nH]1] (69% of theory; reaction time: 16 h) | LC-MS (Method 1): Rₜ = 1.21 min; MS (ESpos): m/z = 385 (M + H)⁺ ¹H NMR (400 MHz, DMSO-d₆): δ = 1.19 (t, 3H), 2.62 (q, 2H), 3.97 (s, 2H), 7.44 (dd, 1H), 7.62 (d, 1H), 7.71 (d, 1H), 13.02-13.24 (m, 1H). |
| 27 | 5-[4-chloro-3-(trifluoromethyl)benzyl]-2- cyclopropyl-6-(trifluoromethyl)pyrimidin-4(3H)- one 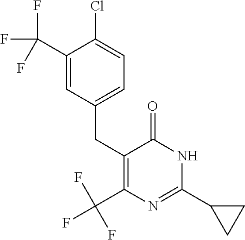 [SMILES: O=c1[nH]c(C2CC2)nc(C(F)(F)F)c1Cc1ccc(Cl)c(C(F)(F)F)c1] (49% of theory; reaction time: 16 h) | LC-MS (Method 1): Rₜ = 1.28 min; MS (ESpos): m/z = 397.2 (M + H)⁺ ¹H NMR (400 MHz, DMSO-d₆): δ = 1.02-1.14 (m, 4H), 1.94-2.02 (m, 1H), 3.94 (s, 2H), 7.43 (d, 1H), 7.61 (d, 1H), 7.70 (d, 1H), 13.34 (br. s, 1H). |
| 28 | 5-(3-chloro-4-fluorobenzyl)-2-ethyl-6- (trifluoromethyl)pyrimidin-4(3H)-one 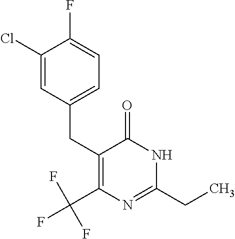 [SMILES: CCc1nc(C(F)(F)F)c(Cc2ccc(F)c(Cl)c2)c(=O)[nH]1] (32% of theory; 3 eq. of propanimidamide hydrochloride; 4 eq. potassium carbonate; dioxane; reaction time: 18 h, reflux) | LC-MS (Method 1): Rₜ = 1.11 min; MS (ESpos): m/z = 335.2 (M + H)⁺ ¹H NMR (400 MHz, DMSO-d₆): δ = 1.20 (t, 3H), 2.62 (q, 2H), 3.89 (s, 2H), 6.95-7.55 (m, 3H), 13.09 (br. s, 1H). |
| 29 | 5-(3-chloro-4-methylbenzyl)-2-cyclopropyl-6- (trifluoromethyl)pyrimidin-4(3H)-one 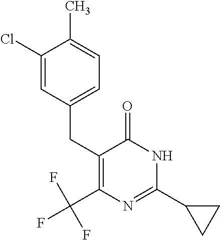 [SMILES: Cc1ccc(Cc2c(C(F)(F)F)nc(C3CC3)[nH]c2=O)cc1Cl] (32% of theory; 3 eq. of cyclopropane-1- carboximidamide hydrochloride; 4 eq. potassium carbonate; dioxane; reaction time: 18 h, reflux) | conditions of prep. HPLC purification: column: Daicel Chiracel OZ-H 5 μm, 250 × 20 mm; flow rate: 15 ml/min; run time: 12 min; detection: 220 nm; mobile phase: isohexane/ethanol 95:5. LC-MS (Method 1): Rₜ = 1.23 min; MS (ESpos): m/z = 343.1 (M + H)⁺ ¹H NMR (400 MHz, DMSO-d₆): δ = 0.97-1.15 (m, 4H), 1.91-2.04 (m, 1H), 3.82 (s, 2H), 6.91-6.98 (m, 1H), 7.11 (s, 1H), 7.28 (d, 1H), 13.27 (br. s, 1H). |
| 30 | 2-cyclopropyl-5-(3,4-dichlorobenzyl)-6- (trifluoromethyl)pyrimidin-4(3H)-one 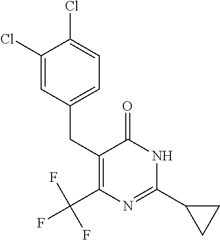 [SMILES: O=c1[nH]c(C2CC2)nc(C(F)(F)F)c1Cc1ccc(Cl)c(Cl)c1] (10% of theory; preparation analogous to Example 25A; base: sodium methoxide; solvent: methanol; reaction time: 10 h, 64° C.) | LC-MS (Method 1): Rₜ = 1.26 min; MS (ESpos): m/z = 363 (M + H)⁺ ¹H NMR (400 MHz, DMSO-d₆): δ = 1.02-1.11 (m, 4H), 1.93-2.02 (m, 1H), 3.86 (s, 2H), 7.12 (dd, 1H), 7.43 (d, 1H), 7.52 (d, 1H), 13.12-13.39 (m, 1H). |
| 31 | 5-(3,4-dichlorobenzyl)-2-ethyl-6- (trifluoromethyl)pyrimidin-4(3H)-one 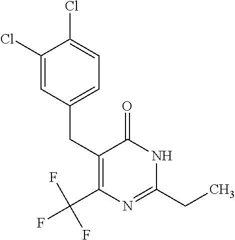 [SMILES: CCc1nc(C(F)(F)F)c(Cc2ccc(Cl)c(Cl)c2)c(=O)[nH]1] (64% of theory; preparation analogous to Example 25A; base: sodium methoxide; solvent: methanol; reaction time: 10 h, 64° C.) | LC-MS (Method 1): Rₜ = 1.18 min; MS (ESpos): m/z = 351 (M + H)⁺ ¹H NMR (400 MHz, DMSO-d₆): δ = 1.20 (t, 3H), 2.62 (q, 2H), 3.90 (s, 2H), 7.13 (dd, 1H), 7.43 (d, 1H), 7.53 (d, 1H), 13.10 (br. s, 1H). |
| 32 | 5-(4-chloro-3-fluorobenzyl)-2-ethyl-6- (trifluoromethyl)pyrimidin-4(3H)-one 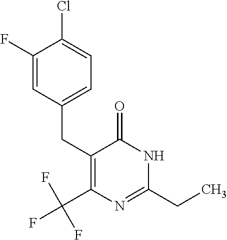 [SMILES: CCc1nc(C(F)(F)F)c(Cc2ccc(Cl)c(F)c2)c(=O)[nH]1] (56% of theory; reaction time: 16 h) | LC-MS (Method 1): Rₜ = 1.12 min; MS (ESpos): m/z = 335 (M + H)⁺ ¹H NMR (400 MHz, DMSO-d₆): δ = 1.20 (t, 3H), 2.62 (q, 2H), 3.91 (s, 2H), 7.02 (d, 1H), 7.21 (d, 1H), 7.48 (t, 1H), 13.04 (s, 1H). |
| 33 | 5-(3,4-dichlorophenoxy)-2-ethyl-6- (trifluoromethyl)pyrimidin-4(3H)-one 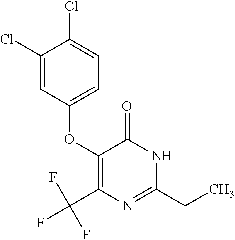 [SMILES: CCc1nc(C(F)(F)F)c(Oc2ccc(Cl)c(Cl)c2)c(=O)[nH]1] (54% of theory; reaction time: 16 h) | LC-MS (Method 1): Rₜ = 1.14 min; MS (ESpos): m/z = 353 (M + H)⁺ ¹H NMR (400 MHz, DMSO-d₆): δ = 1.23 (t, 3H), 2.64 (q, 2H), 7.09 (dd, 1H), 7.42 (d, 1H), 7.57 (d, 1H), 13.37 (br. s, 1H). |
| 34 | 5-(3-chloro-4-fluorobenzyl)-2-cyclopropyl-6- (trifluoromethyl)pyrimidin-4(3H)-one 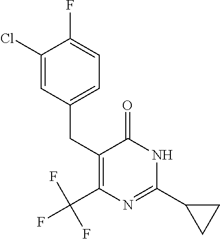 [SMILES: O=c1[nH]c(C2CC2)nc(C(F)(F)F)c1Cc1ccc(F)c(Cl)c1] (38% of theory; 3 eq. of cyclopropane-1- carboximidamide hydrochloride; 4 eq. potassium carbonate; dioxane; reaction time: 18 h, reflux) | LC-MS (Method 1): Rₜ = 1.19 min; MS (ESpos): m/z = 347.2 (M + H)⁺ ¹H NMR (400 MHz, DMSO-d₆): δ = 0.96-1.17 (m, 4H), 1.89-2.07 (m, 1H), 3.34 (s, 2H), 7.09-7.18 (m, 1H), 7.30 (t, 1H), 7.37 (dd, 1H), 13.31 (br. s, 1H). |

### Example 35

### 2-{5-[3-Chloro-4-(trifluoromethyl)benzyl]-6-oxo-4-(trifluoromethyl)-1,6-dihydropyrimidin-2-yl}acetamide

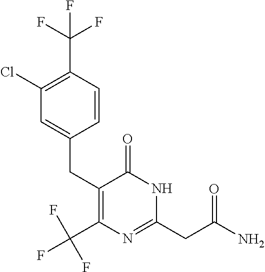 [SMILES: NC(=O)Cc1nc(C(F)(F)F)c(Cc2ccc(C(F)(F)F)c(Cl)c2)c(=O)[nH]1]

A mixture of 293 mg (2.1 mmol) of potassium carbonate, 219 mg (1.6 mmol) of 3,3-diaminoprop-2-enamide hydrochloride and 200 mg (0.5 mmol) of ethyl 2-[3-chloro-4-(trifluoromethyl)benzyl]-4,4,4-trifluoro-3-oxobutanoate (Example 24A) in 2.3 ml of dioxane was heated under reflux for 18 h. The mixture was then filtered, the residue was washed with dioxane and the filtrate was purified by preparative HPLC (mobile phase: acetonitrile/water gradient with 0.1% of formic acid). This gave, from two reactions with, in total, 0.66 mmol of ethyl 2-[3-chloro-4-(trifluoromethyl)benzyl]-4,4,4-trifluoro-3-oxobutanoate, 60 mg (20% of theory) of the title compound.

LC-MS (Method 1): Rₜ=1.01 min; MS (ESpos): m/z=414.1 (M+H)⁺

¹H NMR (400 MHz, DMSO-d₆): δ=3.54 (s, 2H), 4.00 (s, 2H), 7.22-7.34 (m, 2H), 7.54 (s, 1H), 7.65 (br. s, 1H), 7.78 (d, 1H), 13.21 (br. s, 1H).

The exemplary compounds listed in Table 11 were prepared analogously to Example 35 by reacting 3,3-diaminoprop-2-enamide hydrochloride with the appropriate benzyl- or phenoxy-substituted trifluoromethyl keto esters:

TABLE 11

| Example | IUPAC name/structure | |
|---|---|---|
| No. | (yield, reaction conditions) | Analytical data |
| 36 | 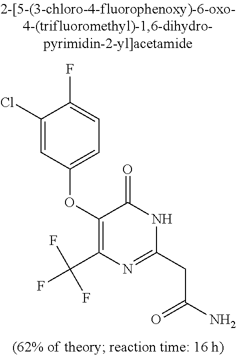 [SMILES: NC(=O)Cc1nc(C(F)(F)F)c(Oc2ccc(F)c(Cl)c2)c(=O)[nH]1] | LC-MS (Method 1): Rₜ = 0.90 min; MS (ESpos): m/z = 366 (M + H)⁺ ¹H NMR (400 MHz, DMSO-d₆): δ = 3.55 (s, 2H), 7.06 (dt, 1H), 7.29 (br. s, 1H), 7.34 (dd, 1H), 7.39 (t, 1H), 7.62 (br. s, 1H), 13.47 (s, 1H). |
| 37 | 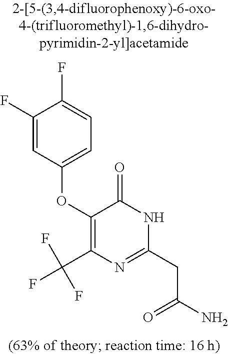 [SMILES: NC(=O)Cc1nc(C(F)(F)F)c(Oc2ccc(F)c(F)c2)c(=O)[nH]1] | LC-MS (Method 1): Rₜ = 0.86 min; MS (ESpos): m/z = 350 (M + H)⁺ ¹H NMR (400 MHz, DMSO-d₆): δ = 3.55 (s, 2H), 6.83-6.91 (m, 1H), 7.22- 7.34 (m, 2H), 7.40 (q, 1H), 7.62 (br. s, 1H), 13.47 (br. s, 1H). |
| 38 | 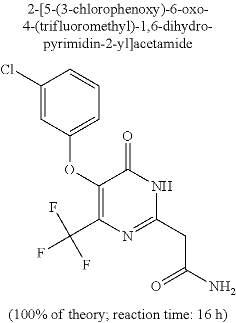 [SMILES: NC(=O)Cc1nc(C(F)(F)F)c(Oc2cccc(Cl)c2)c(=O)[nH]1] | LC-MS (Method 1): Rₜ = 0.88 min; MS (ESpos): m/z = 348 (M + H)⁺ ¹H NMR (400 MHz, DMSO-d₆): δ = 3.56 (s, 2H), 6.99 (dd, 1H), 7.10-7.19 (m, 2H), 7.29 (br. s, 1H), 7.36 (t, 1H), 7.63 (br. s, 1H), 13.47 (br. s, 1H). |
| 39 | 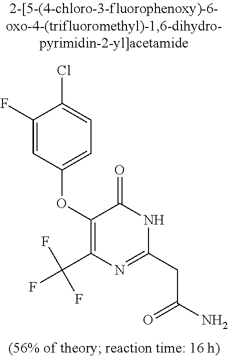 [SMILES: NC(=O)Cc1nc(C(F)(F)F)c(Oc2ccc(Cl)c(F)c2)c(=O)[nH]1] | LC-MS (Method 1): Rₜ = 0.92 min; MS (ESpos): m/z = 366 (M + H)⁺ ¹H NMR (400 MHz, DMSO-d₆): δ = 3.54 (s, 2H), 6.91 (dt, 1H), 7.25 (dd, 1H), 7.28 (br. s, 1H), 7.53 (t, 1H), 7.62 (br. s, 1H), 13.26-13.63 (m, 1H). |
| 40 | 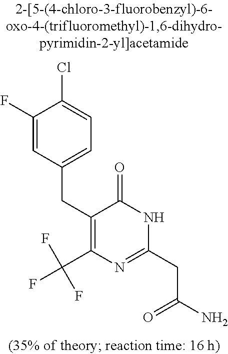 [SMILES: NC(=O)Cc1nc(C(F)(F)F)c(Cc2ccc(Cl)c(F)c2)c(=O)[nH]1] | LC-MS (Method 1): Rₜ = 0.94 min; MS (ESpos): m/z = 364 (M + H)⁺ ¹H NMR (400 MHz, DMSO-d₆): δ = 3.53 (s, 2H), 3.92 (s, 2H), 7.00 (d, 1H), 7.17-7.25 (m, 2H), 7.48 (t, 1H), 7.60-7.64 (m, 1H), 13.05-13.23 (m, 1H). |
| 41 | 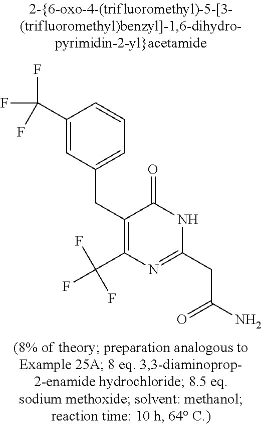 [SMILES: NC(=O)Cc1nc(C(F)(F)F)c(Cc2cccc(C(F)(F)F)c2)c(=O)[nH]1] | LC-MS (Method 1): Rₜ = 0.93 min; MS (ESpos): m/z = 380 (M + H)⁺ ¹H NMR (400 MHz, DMSO-d₆): δ = 3.53 (s, 2H), 4.00 (s, 2H), 7.24 (s, 1H), 7.41-7.48 (m, 1H), 7.48-7.59 (m, 3H), 7.65 (s, 1H), 12.90-13.29 (m, 1H). |
| 42 | 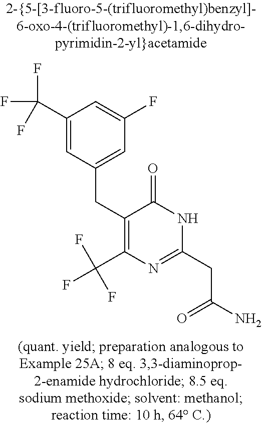 [SMILES: NC(=O)Cc1nc(C(F)(F)F)c(Cc2cc(F)cc(C(F)(F)F)c2)c(=O)[nH]1] | LC-MS (Method 1): Rₜ = 0.96 min; MS (ESpos): m/z = 398 (M + H)⁺ ¹H NMR (400 MHz, DMSO-d₆): δ = 3.53 (s, 2H), 4.02 (s, 2H), 7.26 (s, 1H), 7.32 (d, 1H), 7.42 (s, 1H), 7.53 (d, 1H), 7.65 (s, 1H), 13.20 (s, 1H). |
| 43 | 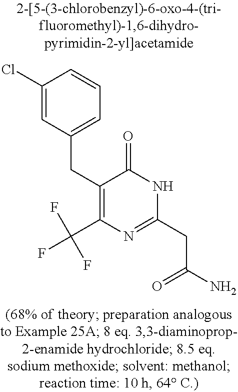 [SMILES: NC(=O)Cc1nc(C(F)(F)F)c(Cc2cccc(Cl)c2)c(=O)[nH]1] | LC-MS (Method 1): Rₜ = 0.91 min; MS (ESpos): m/z = 346 (M + H)⁺ ¹H NMR (400 MHz, DMSO-d₆): δ = 3.52 (s, 2H), 3.92 (s, 2H), 7.10 (d, 1H), 7.21-7.27 (m, 3H), 7.31 (q, 1H), 7.64 (s, 1H), 13.14 (s, 1H). |
| 44 | 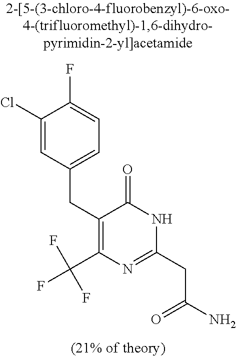 [SMILES: NC(=O)Cc1nc(C(F)(F)F)c(Cc2ccc(F)c(Cl)c2)c(=O)[nH]1] | LC-MS (Method 1): Rₜ = 0.91 min; MS (ESpos): m/z = 364.2 (M + H)⁺ ¹H NMR (400 MHz, DMSO-d₆): δ = 3.53 (s, 2H), 3.90 (s, 2H), 7.11-7.43 (m, 4H), 7.64 (br. s, 1H), 13.17 (br. s, 1H). |
| 45 | 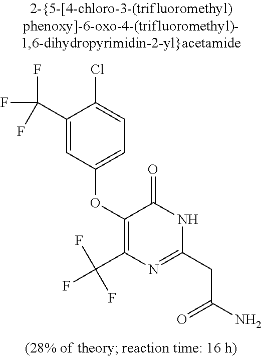 [SMILES: NC(=O)Cc1nc(C(F)(F)F)c(Oc2ccc(Cl)c(C(F)(F)F)c2)c(=O)[nH]1] | LC-MS (Method 1): Rₜ = 0.98 min; MS (ESpos): m/z = 416 (M + H)⁺ ¹H NMR (400 MHz, DMSO-d₆): δ = 3.57 (s, 2H), 7.30 (br. s, 1H), 7.36 (dd, 1H), 7.54 (d, 1H), 7.63 (br. s, 1H), 7.69 (d, 1H), 13.54 (br. s, 1H). |
| 46 | 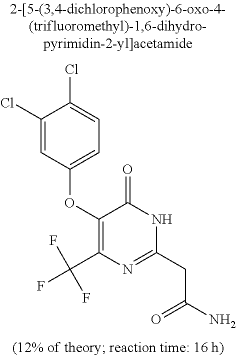 [SMILES: NC(=O)Cc1nc(C(F)(F)F)c(Oc2ccc(Cl)c(Cl)c2)c(=O)[nH]1] | LC-MS (Method 1): Rₜ = 0.96 min; MS (ESpos): m/z = 382 (M + H)⁺ ¹H NMR (400 MHz, DMSO-d₆): δ = 3.55 (s, 2H), 7.07 (dd, 1H), 7.30 (br. s, 1H), 7.41 (d, 1H), 7.59 (d, 1H), 7.62 (br. s, 1H), 13.50 (s, 1H). |
| 47 | 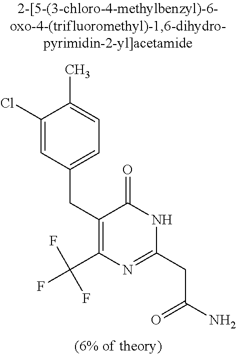 [SMILES: Cc1ccc(Cc2c(C(F)(F)F)nc(CC(N)=O)[nH]c2=O)cc1Cl] | conditions of prep. HPLC purification: column: Daicel Chirapak AS-H 5 μm, 250 x 20 mm; flow rate: 20 ml/min; run time: 7 min; detection: 285 nm; mobile phase: isohexane/(ethanol + 0.1% TFA) 50:50. LC-MS (Method 2): Rₜ = 2.77 min; MS (ESpos): m/z = 360.1 (M + H)⁺ ¹H NMR (400 MHz, DMSO-d₆): δ = 2.26 (s, 3H), 3.52 (s, 2H), 3.87 (s, 2H), 7.01 (d, 1H), 7.17-7.27 (m, 3H), 7.64 (br. s, 1H), 13.13 (br. s, 1H). |
| 48 | 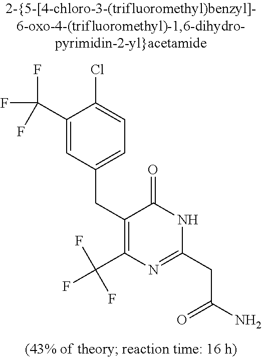 [SMILES: NC(=O)Cc1nc(C(F)(F)F)c(Cc2ccc(Cl)c(C(F)(F)F)c2)c(=O)[nH]1] | LC-MS (Method 1): Rₜ = 1.00 min; MS (ESpos): m/z = 414 (M + H)⁺ ¹H NMR (400 MHz, DMSO-d₆): δ = 3.52 (s, 2H), 3.98 (s, 2H), 7.25 (s, 1H), 7.43 (d, 1H), 7.62-7.66 (m, 2H), 7.70-7.72 (m, 1H), 13.19 (s, 1H). |
| 49 | 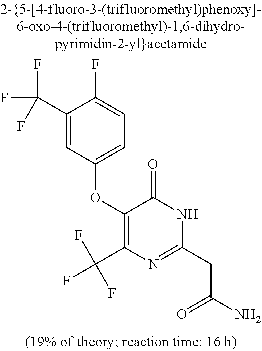 [SMILES: NC(=O)Cc1nc(C(F)(F)F)c(Oc2ccc(F)c(C(F)(F)F)c2)c(=O)[nH]1] | LC-MS (Method 1): Rₜ = 0.93 min; MS (ESpos): m/z = 400 (M + H)⁺ ¹H NMR (400 MHz, DMSO-d₆): δ = 3.56 (s, 2H), 7.29 (br. s, 1H), 7.38- 7.46 (m, 2H), 7.48 (t, 1H), 7.63 (br. s, 1H), 13.50 (br. s, 1H). |
| 50 | 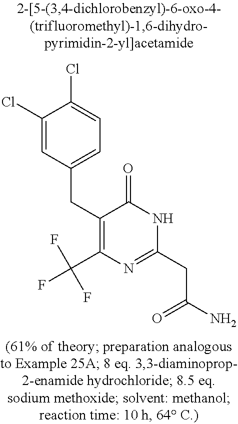 [SMILES: NC(=O)Cc1nc(C(F)(F)F)c(Cc2ccc(Cl)c(Cl)c2)c(=O)[nH]1] | LC-MS (Method 1): Rₜ = 0.96 min; MS (ESpos): m/z = 380 (M + H)⁺ ¹H NMR (400 MHz, DMSO-d₆): δ = 3.52 (s, 2H), 3.91 (s, 2H), 7.13 (dd, 1H), 7.25 (br. s, 1H), 7.43 (d, 1H), 7.54 (d, 1H), 7.65 (br. s, 1H), 13.14 (s, 1H). |

### Example 51

### 5-(3,4-Dichlorobenzyl)-2-(hydroxymethyl)-6-(trifluoromethyl)pyrimidin-4(3H)-one

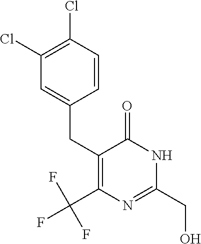 [SMILES: O=c1[nH]c(CO)nc(C(F)(F)F)c1Cc1ccc(Cl)c(Cl)c1]

A mixture of 25 g (75 mmol) of ethyl 2-(3,4-dichlorobenzyl)-4,4,4-trifluoro-3-oxobutanoate [CAS 179110-12-4; WO 2012/041817, Intermediate 56], 10 g (90 mmol) of 2-hydroxyethanimidamide hydrochloride and 19.7 ml (113 mmol) of N,N-diisopropylethylamine in 250 ml of DMF was stirred at 100° C. for 3 h. The mixture was then concentrated on a rotary evaporator to half of its original volume and then diluted with ethyl acetate and extracted with water. The organic phase was dried over magnesium sulphate. After filtration and concentration, the residue was purified chromatographically on silica gel (mobile phase cyclohexane/ethyl acetate 3:1→1:1). This gave 9.69 g (36% of theory) of the title compound.

LC-MS (Method 1): Rₜ=1.03 min; MS (ESpos): m/z=353.0 (M+H)⁺

¹H NMR (400 MHz, DMSO-d₆): δ=3.92 (s, 2H), 4.38 (d, 2H), 5.74 (t, 1H), 7.13 (dd, 1H), 7.35-7.62 (m, 2H), 12.96 (br. s, 1H).

The exemplary compounds listed in Table 12 were prepared analogously to Example 35 by reacting 2-hydroxyethanimidamide with the appropriate phenoxy-substituted trifluoromethyl keto esters:

TABLE 12

| Exam- | | |
|---|---|---|
| ple | IUPAC name/structure | |
| No. | (yield, reaction conditions) | Analytical data |
| 52 | 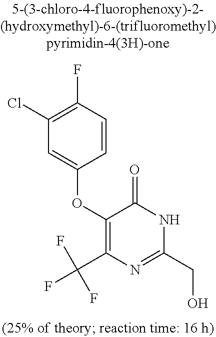 [SMILES: O=c1[nH]c(CO)nc(C(F)(F)F)c1Oc1ccc(F)c(Cl)c1] | LC-MS (Method 1): Rₜ = 0.95 min; MS (ESpos): m/z = 339 (M + H)⁺ ¹H NMR (400 MHz, DMSO-d₆): δ = 4.40 (d, 2H), 5.79 (t, 1H), 7.04-7.11 (m, 1H), 7.34-7.41 (m, 2H), 13.26 (s, 1H). |
| 53 | 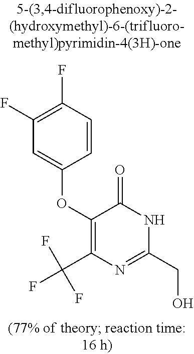 [SMILES: O=c1[nH]c(CO)nc(C(F)(F)F)c1Oc1ccc(F)c(F)c1] | LC-MS (Method 1): Rₜ = 0.90 min; MS (ESpos): m/z = 323 (M + H)⁺ ¹H NMR (400 MHz, DMSO-d₆): δ = 4.40 (d, 2H), 5.80 (t, 1H), 6.82-6.93 (m, 1H), 7.29 (m, 1H), 7.39 (q, 1H), 13.24 (br. s, 1H). |
| 54 | 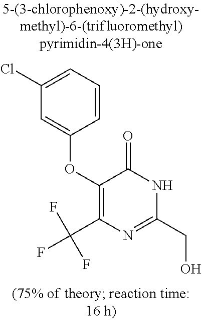 [SMILES: O=c1[nH]c(CO)nc(C(F)(F)F)c1Oc1cccc(Cl)c1] | LC-MS (Method 1): Rₜ = 0.93 min; MS (ESpos): m/z = 321 (M + H)⁺ ¹H NMR (400 MHz, DMSO-d₆): δ = 4.40 (d, 2H), 5.79 (t, 1H), 6.94-7.04 (m, 1H), 7.13-7.18 (m, 2H), 7.32-7.39 (m, 1H), 13.24 (br. s, 1H). |
| 55 | 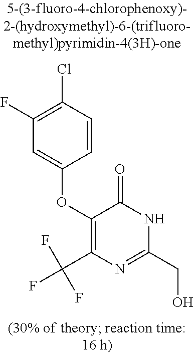 [SMILES: O=c1[nH]c(CO)nc(C(F)(F)F)c1Oc1ccc(Cl)c(F)c1] | LC-MS (Method 1): Rₜ = 0.96 min; MS (ESpos): m/z = 339 (M + H)⁺ ¹H NMR (400 MHz, DMSO-d₆): δ = 4.41 (d, 2H), 5.81 (t, 1H), 6.95 (dt, 1H), 7.29 (dd, 1H), 7.53 (t, 1H), 13.27 (br. s, 1H). |

### Example 56

### 1-[5-(3,4-Dichlorobenzyl)-6-oxo-4-(trifluoromethyl)-1,6-dihydropyrimidin-2-yl]urea

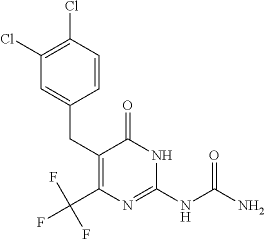 [SMILES: NC(=O)Nc1nc(C(F)(F)F)c(Cc2ccc(Cl)c(Cl)c2)c(=O)[nH]1]

A mixture of 502 mg (3.6 mmol) of potassium carbonate, 320 mg (2.2 mmol) of 1H-pyrazole-1-carboximidamide hydrochloride and 200 mg (0.5 mmol) of ethyl 2-(3,4-dichlorobenzyl)-4,4,4-trifluoro-3-oxobutanoate [CAS 179110-12-4; WO 2012/041817, Intermediate 56) in 5.9 ml of dioxane was stirred at 85° C. for 1 h. 1 ml of 1 N hydrochloric acid was then added, and the mixture was concentrated under reduced pressure. The residue was purified by preparative HPLC (mobile phase: acetonitrile/water gradient with 0.1% of formic acid). 31 mg (11% of theory) of the title compound were obtained as a byproduct of the reaction.

LC-MS (Method 1): Rₜ=1.06 min; MS (ESpos): m/z=381 (M+H)⁺

¹H NMR (400 MHz, DMSO-d₆): δ=3.82 (s, 2H), 6.40 (br. s, 1H), 7.15 (dd, 1H), 7.31-7.48 (br. s, 1H), 7.42 (d, 1H), 7.52 (d, 1H), 10.48 (br. s, 1H), 12.38 (br. s, 1H).

The exemplary compound listed in Table 13 was prepared analogously to Example 2 by reacting guanidine hydrochloride with the appropriate phenoxy-substituted trifluoromethyl keto ester:

TABLE 13

| Exam- | | |
|---|---|---|
| ple | IUPAC name/structure | |
| No. | (yield, reaction conditions) | Analytical data |
| 57 | 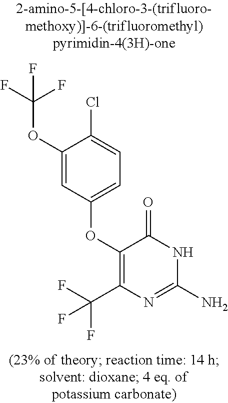 [SMILES: Nc1nc(C(F)(F)F)c(Oc2ccc(Cl)c(OC(F)(F)F)c2)c(=O)[nH]1] | LC-MS (Method 4): Rₜ = 2.01 min; MS (ESpos): m/z = 389.9 (M + H)⁺ ¹H NMR (400 MHz, DMSO-d₆): δ = 7.04- 7.10 (m, 3H), 7.24 (d, 1H), 7.58 (d, 1H), 11.84 (br. s, 1H). |

The exemplary compounds listed in Table 14 were prepared analogously to Example 2 by reacting guanidine hydrochloride with the appropriate pyridylmethyl-substituted trifluoromethyl keto esters:

TABLE 14

| Exam- | | |
|---|---|---|
| ple | IUPAC name/structure | |
| No. | (yield, reaction conditions) | Analytical data |
| 58 | 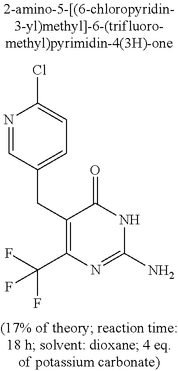 [SMILES: Nc1nc(C(F)(F)F)c(Cc2ccc(Cl)nc2)c(=O)[nH]1] | LC-MS (Method 6): Rₜ = 0.98 min; MS (ESpos): m/z = 305.0 (M + H)⁺ ¹H NMR (400 MHz, DMSO-d₆): δ = 3.75 (s, 2H), 6.09 (br. s, 2H), 7.39 (d, 1H), 7.59 (dd, 1H), 8.22 (d, 1H), 11.54 (br. s, 1H). |
| 59 | 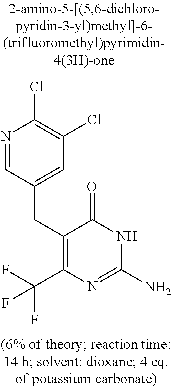 [SMILES: Nc1nc(C(F)(F)F)c(Cc2cnc(Cl)c(Cl)c2)c(=O)[nH]1] | LC-MS (Method 1): Rₜ = 0.88 min; MS (ESpos): m/z = 339.1 (M + H)⁺ ¹H NMR (400 MHz, DMSO-d₆): δ = 3.78 (s, 2H), 6.99 (br. s, 2H), 7.87 (s, 1H), 8.20 (s, 1H), 11.51 (br. s, 1H). |

### Example 60

### 2-Amino-5-{[4-chloro-3-(trifluoromethyl)phenyl]sulphinyl}-6-(trifluoromethyl)pyrimidin-4(3H)-one

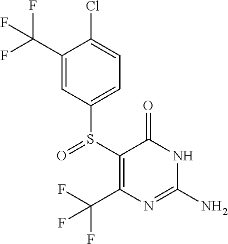 [SMILES: Nc1nc(C(F)(F)F)c(S(=O)c2ccc(Cl)c(C(F)(F)F)c2)c(=O)[nH]1]

At room temperature, 29 mg (74 μmol) of 2-amino-5-{[4-chloro-3-(trifluoromethyl)phenyl]sulphanyl}-6-(trifluoromethyl)pyrimidin-4(3H)-one (Example 17) were dissolved in 1.5 ml of acetic acid, and 30 μl of hydrogen peroxide (30% by weight in water) were added. The reaction mixture was stirred at 45° C. for 4 h. After addition of 1 ml of N,N-dimethylformamide, the mixture was purified directly by preparative HPLC (mobile phase: acetonitrile/water gradient with 0.1% of formic acid). The product-containing fractions were concentrated and the residue was dried under reduced pressure. This gave 20 mg (66% of theory) of the title compound.

LC-MS (Method 1): Rₜ=0.89 min; MS (ESpos): m/z=406.0 (M+H)⁺

¹H NMR (400 MHz, DMSO-d₆): δ=7.14 (br. s, 1H), 7.75 (dd, 1H), 7.83 (d, 1H), 7.99 (d, 1H), 8.65 (br. s, 1H), 11.73 (br. s, 1H).

The exemplary compounds listed in Table 15 were prepared analogously to Example 60:

TABLE 15

| Exam- | | |
|---|---|---|
| ple | IUPAC name/structure | |
| No. | (yield) | Analytical data |
| 61 | 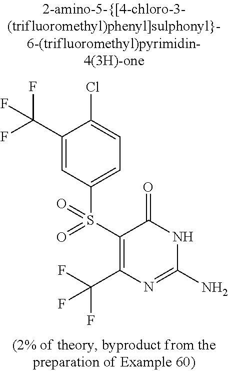 [SMILES: Nc1nc(C(F)(F)F)c(S(=O)(=O)c2ccc(Cl)c(C(F)(F)F)c2)c(=O)[nH]1] | LC-MS (Method 1): Rₜ = 0.95 min; MS (ESpos): m/z = 421.9 (M + H)⁺ |
| 62 | 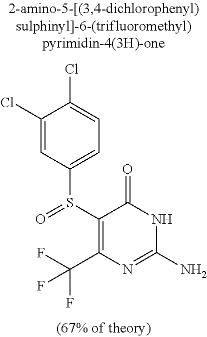 [SMILES: Nc1nc(C(F)(F)F)c(S(=O)c2ccc(Cl)c(Cl)c2)c(=O)[nH]1] | LC-MS (Method 1): Rₜ = 0.85 min; MS (ESpos): m/z = 372.0 (M + H)^{⁺} ¹H NMR (400 MHz, DMSO-d₆): δ = 7.12 (br. s, 1H), 7.44 (dd, 1H), 7.73-7.75 (m, 2H), 8.59 (br. s, 1H), 11.65 (br. s, 1H). |
| 63 | 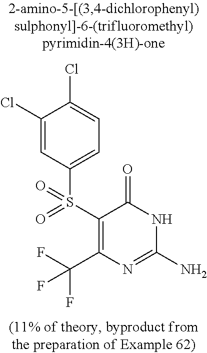 [SMILES: Nc1nc(C(F)(F)F)c(S(=O)(=O)c2ccc(Cl)c(Cl)c2)c(=O)[nH]1] | LC-MS (Method 1): Rₜ = 0.88 min; MS (ESpos): m/z = 388.0 (M + H)⁺ |

### Example 64

### 5-(3,4-Dichlorophenoxy)-2-(ethylamino)-6-(trifluoromethyl)pyrimidin-4(3H)-one

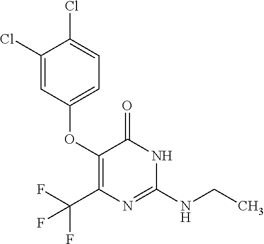 [SMILES: CCNc1nc(C(F)(F)F)c(Oc2ccc(Cl)c(Cl)c2)c(=O)[nH]1]

At −78° C., 58 μl (1.0 mmol) of glacial acetic acid were added to 1.0 ml (1.0 mmol) of a 1 M solution of ethylamine in THF and 5 pellets of molecular sieve (4 Å), and the mixture was then warmed to 0° C. 50 mg (0.1 mmol) of 5-(3,4-dichlorophenoxy)-2-(methylsulphonyl)-6-(trifluoromethyl)pyrimidin-4(3H)-one were then added, and the reaction mixture was heated in a microwave apparatus at 150° C. for 1.5 h. The reaction mixture was then filtered and the filtrate was purified by preparative HPLC (mobile phase: acetonitrile/water gradient with 0.1% trifluoroacetic acid). The product-containing fractions were concentrated and the residue was dried under reduced pressure. This gave 45 mg (99% of theory) of the title compound.

LC-MS (Method 1): Rₜ=1.12 min; MS (ESpos): m/z=368.1 (M+H)⁺

¹H NMR (400 MHz, DMSO-d₆): δ=1.13 (t, 3H), 3.30 (q, 2H), 6.92 (br. s, 1H), 7.01 (dd, 1H), 7.32 (d, 1H), 7.52 (d, 1H), 11.74 (br. s, 1H).

The exemplary compound listed in Table 16 was prepared analogously to Example 64:

TABLE 16

| Exam- | | |
|---|---|---|
| ple | IUPAC name/structure | |
| No. | (yield) | Analytical data |
| 65 | 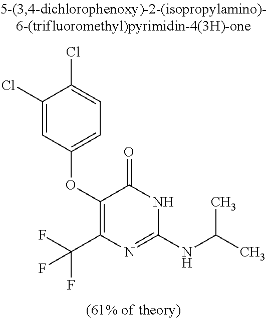 [SMILES: CC(C)Nc1nc(C(F)(F)F)c(Oc2ccc(Cl)c(Cl)c2)c(=O)[nH]1] | LC-MS (Method 1): Rₜ = 1.17 min; MS (ESpos): m/z = 382.1 (M + H)⁺ ¹H NMR (400 MHz, DMSO- d₆): δ = 1.17 (d, 6H), 4.00 (sept, 1H), 6.79 (br. s, 1H), 7.01 (dd, 1H), 7.31 (d, 1H), 7.52 (d, 1H), 11.45 (br. s, 1H). |

The exemplary compounds listed in Table 17 were prepared analogously to Example 2 or Example 25A by reacting the respective guanidines or amidines (carboximidamides) or their salts with the appropriate substituted trifluoromethyl keto esters:

TABLE 17

| Exam- | | |
|---|---|---|
| ple | IUPAC name/structure | |
| No. | (yield, reaction conditions) | Analytical data |
| 66 | 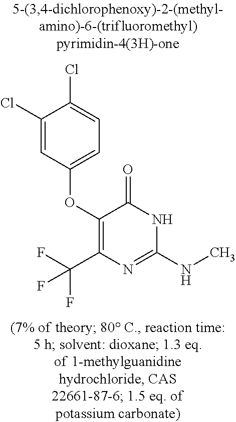 [SMILES: CNc1nc(C(F)(F)F)c(Oc2ccc(Cl)c(Cl)c2)c(=O)[nH]1] | LC-MS (Method 1): Rₜ = 1.06 min; MS (ESpos): m/z = 354.0 (M + H)⁺ ¹H NMR (400 MHz, DMSO-d₆): δ = 2.82 (s, 3H), 6.87 (br. s, 1H), 7.02 (dd, 1H), 7.31 (d, 1H), 7.53 (d, 1H), 11.94 (br. s, 1H). |
| 67 | 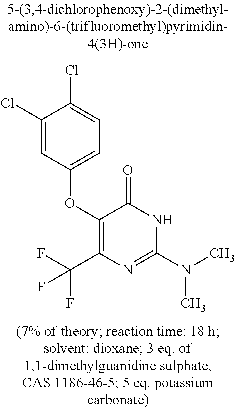 [SMILES: CN(C)c1nc(C(F)(F)F)c(Oc2ccc(Cl)c(Cl)c2)c(=O)[nH]1] | LC-MS (Method 1): Rₜ = 1.23 min; MS (ESpos): m/z = 366.1 (M + H)⁺ ¹H NMR (400 MHz, DMSO-d₆): δ = 3.07 (s, 6H), 7.11 (dd, 1H), 7.38 (d, 1H), 7.51 (d, 1H), 11.62 (br. s, 1H). |
| 68 | 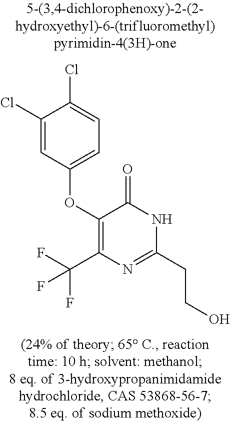 [SMILES: O=c1[nH]c(CCO)nc(C(F)(F)F)c1Oc1ccc(Cl)c(Cl)c1] | LC-MS (Method 1): Rₜ = 1.01 min; MS (ESpos): m/z = 367.1 (M + H)⁺ ¹H NMR (400 MHz, DMSO-d₆): δ = 2.72- 2.77 (m, 2H), 3.73-3.81 (m, 2H), 4.82 (br. s, 1H), 7.14 (dd, 1H), 7.43 (d, 1H), 7.54 (d, 1H), 13.10 (br. s, 1H). |
| 69 | 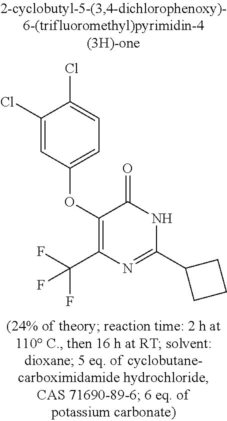 [SMILES: O=c1[nH]c(C2CCC2)nc(C(F)(F)F)c1Oc1ccc(Cl)c(Cl)c1] | LC-MS (Method 1): Rₜ = 1.20 min; MS (ESpos): m/z = 379.1 (M + H)⁺ ¹H NMR (400 MHz, DMSO-d₆): δ = 1.77- 1.90 (m, 1H), 1.93-2.07 (m, 1H), 2.25 (br. s, 2H), 2.32-2.44 (m, 2H), 3.45-3.58 (m, 1H), 7.09 (dd, 1H), 7.43 (d, 1H), 7.56 (d, 1H), 13.24 (br. s, 1H). |
| 70 | 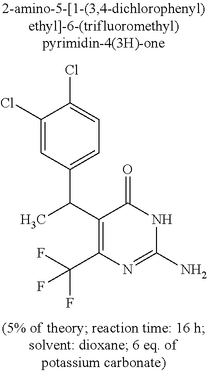 [SMILES: CC(c1ccc(Cl)c(Cl)c1)c1c(C(F)(F)F)nc(N)[nH]c1=O] | LC-MS (Method 1): Rₜ = 1.06 min; MS (ESpos): m/z = 352.1 (M + H)⁺ ¹H NMR (500 MHz, CD₃OD): δ = 1.64 (d, 3H), 4.17 (q, 1H), 7.18- 7.21 (m, 1H), 7.37 (d, 1H), 7.42-7.45 (m, 1H). |
| 71 | 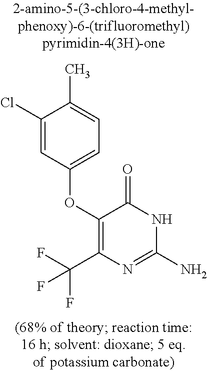 [SMILES: Cc1ccc(Oc2c(C(F)(F)F)nc(N)[nH]c2=O)cc1Cl] | LC-MS (Method 1): Rₜ = 0.93 min; MS (ESpos): m/z = 320.1 (M + H)⁺ ¹H NMR (400 MHz, DMSO-d₆): δ = 2.25 (s, 3H), 6.83 (dd, 1H), 7.00 (br. s, 3H), 7.24 (d, 1H), 11.76 (br. s, 1H). |
| 72 | 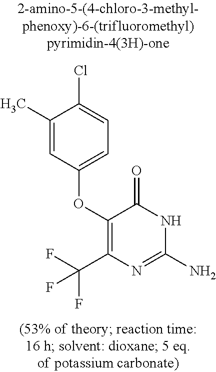 [SMILES: Cc1cc(Oc2c(C(F)(F)F)nc(N)[nH]c2=O)ccc1Cl] | LC-MS (Method 1): Rₜ = 0.94 min; MS (ESneg): mix = 318.0 (M − H)⁻ ¹H NMR (400 MHz, DMSO- d₆): δ = 2.28 (s, 3H), 6.77 (dd, 1H), 6.91- 7.04 (m, 3H), 7.28 (d, 1H), 11.76 (br. s, 1H). |
| 73 | 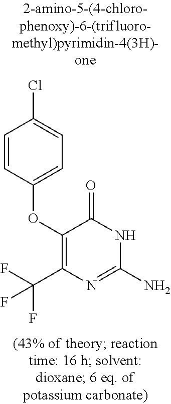 [SMILES: Nc1nc(C(F)(F)F)c(Oc2ccc(Cl)cc2)c(=O)[nH]1] | LC-MS (Method 4): Rₜ = 1.80 min: MS (ESpos): m/z = 306.0 (M + H)⁺ ¹H NMR (400 MHz, DMSO-d₆): δ = 6.90- 7.07 (m, 4H), 7.29- 7.35 (m, 2H), 11.79 (br. s, 1H). |
| 74 | 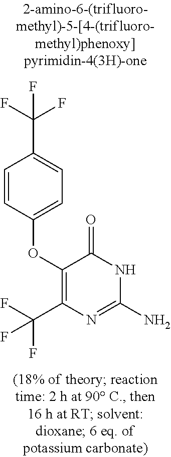 [SMILES: Nc1nc(C(F)(F)F)c(Oc2ccc(C(F)(F)F)cc2)c(=O)[nH]1] | LC-MS (Method 1): Rₜ = 0.93 min; MS (ESneg): m/z = 338.1 (M − H)⁻ ¹H NMR (400 MHz, DMSO-d₆): δ = 6.96- 7.11 (m, 2H), 7.14 (d, 2H), 7.65 (d, 2H), 11.81-12.12 (m, 1H). |
| 75 | 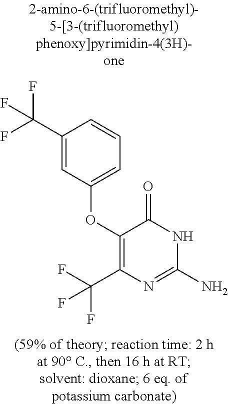 [SMILES: Nc1nc(C(F)(F)F)c(Oc2cccc(C(F)(F)F)c2)c(=O)[nH]1] | LC-MS (Method 1): Rₜ = 0.91 min; MS (ESpos): m/z = 340.1 (M + H)⁺ ¹H NMR (400 MHz, DMSO-d₆): δ = 6.94- 7.10 (m, 2H), 7.24-7.29 (m, 2H), 7.38 (d, 1H), 7.49-7.55 (m, 1H), 11.82 (br. s, 1H). |

### B. ASSESSMENT OF PHARMACOLOGICAL EFFICACY

The pharmacological activity of the compounds according to the invention can be demonstrated by in vitro and in vivo studies, as known to the person skilled in the art. The application examples which follow describe the biological action of the compounds according to the invention, without restricting the invention to these examples.

### Abbreviations and Acronyms:

BSA bovine serum albumin
DMEM Dulbecco's modified Eagle's medium
DMSO dimethyl sulphoxide
FCS foetal calf serum
HEPES 4-(2-hydroxyethyl)piperazine-1-ethanesulphonic acid
LPS lipopolysaccharide(s)
MEM minimum essential medium
PBMC peripheral blood mononuclear cells
PBS phosphate-buffered saline solution
PEG polyethylene glycol
RNA ribonucleic acid(s)
Tris tris(hydroxymethyl)aminomethane
v/v ratio by volume (of a solution)
w/v weight to volume ratio (of a solution)
WBC white blood cells

### B-1. Functional Ca²⁺ Release Test

The antagonistic action of test substances on CCR2 was determined in a functional Ca²⁺ release test. Binding of CCL2/MCP-1 to CCR2 leads to a change in the conformation of the receptor resulting in Gi/Gq protein activation and intracellular signal cascade. This involves, inter alia, an intracellular Ca²⁺ release. The test cell used was a Chem-1 cell line transfected with human CCR2 (ChemiSCREEN™ CCR2B Calcium-Optimized FLIPR Cell Line, Merck Millipore).

The test substances were dissolved in dimethyl sulphoxide (DMSO) at a concentration of 10 mM and serially diluted with DMSO in steps of 1:3.16 for a 10-point dose/activity analysis. According to the desired test concentrations, the substances were pre-diluted in Tyrode with 2 mM CaCl₂ and 0.05% BSA.

The cells, cultivated in DMEM high glucose [supplemented with 10% FCS, 1 mM pyruvate, 15 mM HEPES, 500 μg/ml geniticin and non-essential amino acids (NEAA)], were sown at 5000 cells/25 μl in 384 well, μCLEAR/black cell culture plates from Greiner (#781092) and incubated at 37° C. for 24 h. The sowing medium consisted of DMEM high glucose [supplemented with 5% FCS, 1 mM pyruvate, 15 mM HEPES, 50 U/ml penicillin, 50 μg/ml streptomycin and non-essential amino acids (NEAA)]. The medium was then removed and the cells were charged for 60 min at 37° C. with Fluo-4 dye [25 μl Tyrode with 3 μM Fluo-4 AM (1 mM DMSO stock solution), 0.4 mg/ml Brilliant Black, 2.5 mM probenicid, 0.03% Pluronic F-127]. The cells were pre-incubated for 10 min with 10 μl of the test substances diluted in buffer, and 20 μl of agonist solution (MCP-1 in Tyrode with 0.05% BSA) were then added. MCP-1 was employed at the concentration which corresponds to the EC₅₀ which had been determined in a preliminary test (usually about 5 nM). Ca²⁺ release was monitored over a period of 120 s in 1 s increments in a proprietary fluorescence imaging reader. The molar concentration of the test substance which caused 50% inhibition of the MCP-1 effect (IC₅₀) was determined using a 4-parameter logistic function (Hill function).

The IC₅₀ values determined in this manner from this assay for individual working examples are given in Table 1 below (in some cases as means of a plurality of independent individual determinations):

TABLE 1

| Example No. | IC₅₀ [nM] |
|---|---|
| 1 | 19 |
| 2 | 3.3 |
| 3 | 3.4 |
| 4 | 6.5 |
| 5 | 8.7 |
| 6 | 5.4 |
| 7 | 11 |
| 8 | 33 |
| 9 | 38 |
| 10 | 43 |
| 11 | 42 |
| 12 | 54 |
| 13 | 69 |
| 14 | 71 |
| 15 | 2.3 |
| 16 | 31 |
| 17 | 4.1 |
| 18 | 33 |
| 19 | 63 |
| 20 | 300 |
| 21 | 230 |
| 22 | 110 |
| 23 | 280 |
| 24 | 270 |
| 25 | 15 |
| 26 | 31 |
| 27 | 63 |
| 28 | 44 |
| 29 | 91 |
| 30 | 100 |
| 31 | 120 |
| 32 | 220 |
| 33 | 15 |
| 34 | 150 |
| 35 | 150 |
| 36 | 110 |
| 37 | 480 |
| 38 | 320 |
| 39 | 160 |
| 40 | 150 |
| 41 | 270 |
| 42 | 400 |
| 43 | 270 |
| 44 | 90 |
| 45 | 8.0 |
| 46 | 20 |
| 47 | 49 |
| 48 | 31 |
| 49 | 91 |
| 50 | 65 |
| 51 | 14 |
| 52 | 7.8 |
| 53 | 70 |
| 54 | 10 |
| 55 | 6.2 |
| 56 | 17 |
| 57 | 1.8 |
| 58 | 381 |
| 59 | 11 |
| 60 | 117 |
| 61 | 110 |
| 62 | 542 |
| 63 | 326 |
| 64 | 13 |
| 65 | 121 |
| 66 | 9.8 |
| 67 | 55 |
| 68 | 49 |
| 69 | 32 |
| 70 | 45 |
| 71 | 2.4 |
| 72 | 1.5 |
| 73 | 38 |
| 74 | 50 |
| 75 | 21 |

B-2a. Functional 13-Arrestin Recruiting Test with Human MCP-1

The antagonistic action of test substances on CCR2 was determined in a β-arrestin test. The PathHunter β-arrestin GPCR test system (DiscoveRx Corporation, Ltd.) is a cell-based functional method for detecting binding of β-arrestin to an activated receptor. The molecular basis is a β-galactosidase complementation measured by the enzymatic conversion of a chemiluminescent substrate. The test cell used was a U2OS β-arrestin cell line transfected with murine CCR2 (93-0543C₃, DiscoveRx Corporation, Ltd.).

The test substances were dissolved in dimethyl sulphoxide (DMSO) at a concentration of 10 mM and serially diluted with DMSO in steps of 1:3.16 for a 10-point dose/activity analysis. According to the desired test concentrations, the substances were pre-diluted in Tyrode with 2 mM CaCl₂ and 0.05% BSA.

The cells, cultivated in MEM Eagle (supplemented with 10% FCS, 50 U/ml of penicillin, 50 μg/ml of streptomycin, 250 μg/ml of hygromycin and 500 μg/ml of geniticin), were sown at 2000 cells/25 μl in 384 well, μCLEAR/black cell culture plates from Greiner (#781092) and incubated at 37° C. for 24 h. The sowing medium consisted of Opti-MEM (supplemented with 1% FCS, 50 U/ml of penicillin and 50 μg/ml of streptomycin). The cells were pre-incubated for 10 min with 10 μl of the test substances diluted in buffer, and 10 μl of agonist solution [human MCP-1 (PeproTech, #300-04) in Tyrode with 0.05% BSA] were then added. The human MCP-1 was employed at the concentration which corresponds to the EC₅₀ which had been determined in a preliminary test (usually about 3 nM). After 90 min of incubation at 37° C., the solution was removed, and recruitment of β-arrestin to CCR2 was detected with the aid of the PathHunter detection reagent (93-001, DiscoveRx Corporation, Ltd.) according to the instructions of the manufacturer. Luminescence was measured after an incubation time of 60 min using a proprietary luminescence imaging measuring instrument. The molar concentration of the test substance which caused 50% inhibition of the MCP-1 effect (IC₅₀) was determined using a 4-parameter logistic function (Hill function).

The IC₅₀ values determined in this manner from this assay for individual working examples are given in Table 2a below (in some cases as means of a plurality of independent individual determinations):

TABLE 2a

| Example No. | IC₅₀ [nM] |
|---|---|
| 1 | 200 |
| 2 | 23 |
| 3 | 49 |
| 4 | 220 |
| 5 | 210 |
| 6 | 150 |
| 7 | 300 |
| 8 | 160 |
| 9 | 450 |
| 10 | 1300 |
| 11 | 560 |
| 12 | 1000 |
| 13 | 330 |
| 14 | 810 |
| 15 | 130 |
| 16 | 640 |
| 17 | 160 |
| 18 | 370 |
| 19 | 980 |
| 20 | 1200 |
| 21 | 590 |
| 22 | 740 |
| 23 | 1200 |
| 24 | 1900 |
| 25 | 170 |
| 26 | 240 |
| 27 | 360 |
| 28 | 290 |
| 29 | 800 |
| 30 | 550 |
| 31 | 220 |
| 32 | 540 |
| 33 | 74 |
| 34 | 520 |
| 35 | 3700 |
| 36 | 1600 |
| 37 | 3800 |
| 38 | 3300 |
| 39 | 1800 |
| 40 | 970 |
| 41 | 2000 |
| 42 | 2800 |
| 43 | 880 |
| 44 | 420 |
| 45 | 130 |
| 46 | 440 |
| 47 | 830 |
| 48 | 300 |
| 49 | 380 |
| 50 | 460 |
| 51 | 230 |
| 52 | 140 |
| 53 | 620 |
| 54 | 140 |
| 55 | 120 |
| 56 | 330 |
| 67 | 1000 |
| 68 | 370 |

B-2b. Functional β-Arrestin Recruiting Test with Murine MCP-1

The test was carried out in a manner identical to that described above under B-2a, but using murine MCP-1 (PeproTech, #250-10) as agonist.

The IC₅₀ values determined in this way from this assay for individual working examples are given in Table 2b below (in some cases as means of a plurality of independent individual determinations):

TABLE 2b

| Example No. | IC₅₀ [nM] |
|---|---|
| 1 | 312 |
| 2 | 69 |
| 3 | 280 |
| 57 | 2270 |
| 58 | 60000 |
| 59 | 7280 |
| 60 | 1430 |
| 61 | 2950 |
| 62 | 1340 |
| 63 | 2200 |
| 64 | 2700 |
| 65 | 1630 |
| 66 | 506 |
| 69 | 523 |
| 70 | 3710 |
| 71 | 1720 |
| 72 | 545 |
| 73 | 2310 |
| 74 | 4160 |
| 75 | 9490 |

### B-3. Test of Selectivity for Human CC Receptors

The antagonistic effect of test substances on human CC receptors was determined in functional

Ca²⁺ release tests using Ca²⁺-sensitive fluorescent dyes. The test cells used were Chem-1 or Chem-5 cell lines transfected with the respective receptor (ChemiSCREEN™ CCR Calcium-Optimized FLIPR Cell Lines, Merck Millipore; CCR1: HTS005C; CCR3: HTS008C; CCR4: HTS009C; CCR5 rhesus monkey: HTS010C; CCR6: HTS011C; CCR7: HTS012C; CCR8: HTS013C; CCR9: HTS036C; CCR10: HTS014C).

The substance test was carried out in a FLIPR tetra instrument (Molecular Devices). The agonist in question was added in a concentration corresponding to the EC₈₀. Ca²⁺ release was measured over a period of 180 sec.

### B-4. Test of Selectivity for Murine CC Receptors

The antagonistic effect of test substances on murine CC receptors was determined in the PathHunter β-arrestin GPCR test system (DiscoveRx Corporation, Ltd.). The test cells used were U2OS or CHO-K1 β-arrestin cell lines transfected with the respective murine receptor (DiscoveRx Corporation, Ltd.; mCCR1: 93-0561C3; mCCR3: 93-0522C2; mCCR4: 93-0515C2; mCCR5: 93-0470C2; mCCR6: 93-0694C2; mCCR7: 93-0528C2; mCCR8: 93-0556C2; mCCR9: 93-0734C2).

The substance test was carried out with an EnVision microplate reader (Perkin Elmer) which detects the chemiluminescent conversion of the 3-galactosidase substrate. The agonist in question was added in a concentration corresponding to the ECK).

### B-5. Activity Test for CCR2 (Rat) and CCR5 (Rat)

The antagonistic effect of test substances on CCR2 (rat) and CCR5 (rat) was determined in functional Ca²⁺ release tests using the Ca²⁺-sensitive photoprotein aequorin [Vakili et al., J. Immunol. 167, 3406 (2001); Fichna et al., J. Pharmacol. Exp. Ther. 317, 1150 (2006); Silvano et al., Mol. Pharmacol. 78, 925 (2010)]. The test cells used were CHO-K1 cell lines transfected with the respective receptor and aequorin (Euroscreen SA; rCCR2: FAST-0616A; rCCR5: FAST-0617A).

Luminescent detection of Ca²⁺ release was carried out using a Functional Drug Screening System 6000 (FDSS 6000) luminometer (Hamamatsu). The agonist in question was added in a concentration corresponding to the EC₈₀.

### B-6. Thp-1 Migration Assay

The migration of THP-1 cells is analysed using a CytoSelect 96-well cell migration assay (5 μm membrane pores), Fluormetric (BioCat GmbH) or a comparable assay, and the effect of test substances on the migration behaviour is investigated. Alternatively, macrophages are isolated from whole blood (canine, porcine or human) and used for carrying out a migration assay.

### B-7. THP-1 Gene Expression Assay

THP-1 cells are incubated for 7-24 h with 9-cis-retinoic acid to initiate cell differentiation. During the incubation, test substance is added to the medium, and the RNA is then isolated (TRIzol®, Invitrogen). After work-up of the RNA and reverse transcription (ImProm-II™ Reverse Transcription System, Promega A3800), an MCP-1 gene expression analysis is carried out using TaqMan.

### B-8. Human Whole Blood Assay (PBMC Assay)/MCP-1-Induced Gene Expression

The blood is removed into heparin monovettes (Sarstedt) and the blood is then collected and 2.5 ml each are pipetted into the wells of a 12-well plate. 2.5 μl of solvent or test substance solution are pipetted into each well, the contents of the individual wells are mixed for about 5 min on a plate shaker and the plates are then incubated in an incubator at 37° C. for 20 min. The hMCP-1 (100 ng/ml) is then added, followed by about 4 min of mixing on a plate shaker and subsequent incubation in an incubator at 37° C. for 4 h. The blood is then transferred into PAXgene® blood RNA tubes (PreAnalytix) and, after work-up of the RNA and reverse transcription (ImProm-II™ Reverse Transcription System, Promega A3800), a gene expression analysis is carried out using TaqMan.

B-9. Acute Myocardial Infarction (aMI) in the Rat

Male Wistar rats (280-300 g; Harlan Nederland) are anaesthetized with 160 mg/kg of ketamine and 8 mg/kg of xylazine, intubated, connected to a ventilation pump (ugo basile 7025 rodent; 0.4-0.5 litre/min, 60 x/min) and ventilated with 60% compressed air/40% O₂. The body temperature is maintained at 37-38° C. by a heating mat. If appropriate, 0.03 mg/kg s.c. of Temgesic® may be administered as analgesic. The area to be operated on is disinfected (for example with Cutasept), the thorax of the animal is opened between the 3rd and the 4th rib and fixated using a rib spreader. The heart of the animal is exposed under the auricula atrii and a 5-0 Prolene thread is passed underneath about 2 mm from the end of the auricula atrii. Both ends of the thread are pushed into a PE50 plunger and the ends of the thread are coiled around a needle holder. Owing to the resulting tension, the coronary artery of the left ventricle (LAD) is clamped. A bulldog clamp is placed on top of the PE50 plunger and used to occlude the LAD (occlusion time 30 minutes). After this time, the bulldog clamp is loosened and the PE50 plunger is removed; the thread remains in place. The thorax is closed again, and the muscle layers and the epidermis are sutured using coated Vicryl L 5-0 (V990H). Antisedan® i.m. is then injected to reverse anaesthesia.

After 1-4 days of treatment with the test substance, the animals are again anaesthetized (2% isoflurane/compressed air/O₂) and a pressure catheter (Millar SPR-320 2F) is inserted via the carotid artery into the left ventricle after measurement of the systemic blood pressure. The heart rate, left ventricular pressure (LVP), left-ventricular end-diastolic pressure (LVEDP), contractility (dp/dt) and relaxation rate (tau) are measured there and analysed with the aid of the Powerlab system (AD Instruments) and LabChart software. A blood sample is then taken to determine the plasma levels of the substance and plasma biomarkers, and the animals are sacrificed. Area at risk (the non-perfused area) and infarct size are determined by perfusion with Evans Blue (0.2%) and subsequent TTC staining

B-10. Chronic Myocardial Infarction (cMI) in the Rat

Male Wistar rats (280-300 g; Harlan Nederland) are anaesthetized with 5% isoflurane in an anaesthesia cage, intubated, connected to a ventilation pump (ugo basile 7025 rodent; 0.4-0.5 litre/min, 60 x/min) and ventilated with 5% enflurane/compressed air/O₂. The body temperature is maintained at 37-38° C. by a heating mat. If appropriate, 0.03 mg/kg s.c. of Temgesic® may be administered as analgesic. The chest is opened laterally between the third and fourth ribs, and the heart is exposed. The coronary artery of the left ventricle (LAD) is permanently ligated with an occlusion thread (Prolene Ethicon 5-0, EH7401H) passed underneath shortly below its origin (below the left atrium). The thorax is closed again, and the muscle layers and the epidermis are sutured using coated Vicryl L 5-0 (V990H). The surgical suture is wetted with spray dressing (for example Nebacetin® N spray dressing, active ingredient neomycin sulphate), and anaesthesia is then terminated. Alternatively, the occlusion thread may initially be passed around the LAD without occluding it. After closure of the thorax and a healing phase (up to 1 week later), the LAD is then occluded by pulling the occlusion thread, which had been led outside of the body.

The animals are randomized by troponine determination and divided into individual treatment groups and a control group with no substance treatment. A further control included is a sham group in which only the surgical procedure, but not the LAD occlusion, was performed. Treatment with the test substance takes place over 8 weeks by gavage or by adding the test substance to the feed or drinking water.

After treatment for 8 weeks, the animals are again anaesthetized (2% isoflurane/compressed air/O₂) and a pressure catheter (Millar SPR-320 2F) is inserted via the carotid artery into the left ventricle. The heart rate, left ventricular pressure (LVP), left-ventricular end-diastolic pressure (LVEDP), contractility (dp/dt) and relaxation rate (tau) are measured there and analysed with the aid of the Powerlab system (AD Instruments) and LabChart software. A blood sample is then taken to determine the plasma levels of the substance and plasma biomarkers, and the animals are sacrificed. The heart (heart chambers, left ventricle plus septum, right ventricle), liver, lung and kidney are removed and weighed.

### B-11. Acute Lung Injury (ALI) in the Rat

Male Sprague Dawley rats (200-250 g; Charles River) are anaesthetized with 5% isoflurane in an anaesthesia cage. In the tolerance stage, the animals are intubated, with the aid of a guide wire, with a peripheral venous catheter (Brauniile, 16G), and the harmful substance (3 mg/kg of LPS in 100 μl of physiological saline) is administered via the tube. Control animals receive 100 μl of saline. 24 hours after administration of the harmful substance, a pulmonary lavage is carried out. Prior to the lavage, the animals are weighed again to determine the lung index (weight of the lung/body weight). For the lavage, the animals are anaesthetized with isoflurane. The trachea is prepared, and a Braunüle (16G) is inserted and fixed. Via the Braunüle, the lung is rinsed three times with 1.5 ml of physiological saline. The lavage is stored on ice, and the lavages of individual animals are combined and measured on a CellDyn 3700 to determine the number of inflammatory cells (leukocytes, neutrophiles, monocytes).

### B-12. Acute Lung Injury (ALI) in the Mouse

Male mice (Balb/cAnN, about 20 g; Charles River) are anaesthetized with compressed air/oxygen/5% isoflurane. Using a pipette, 100 μl of a solution of the harmful substance to be administered (3 mg/kg of LPS or 10 ng of LPS/MCP-1; see Maus et al., Am. J. Resp. Crit. Care Med. 2001, 164 (3), 406-411) are administered deep into the mouth above the larynx. The animal inhales all of the liquid. 24 to 48 hours after the administration of the harmful substance, pulmonary lavage is carried out. To this end, the mice are anaesthetized again as described above. The thorax is opened and the trachea is exposed. An indwelling cannula (20 G) is introduced into the trachea and fixed with a thread. Via the cannula, 0.5 ml of physiological saline is administered to the lung. This is used to rinse the lung three times. The lavage obtained in this manner is transferred into a vessel. In this manner, the lung is rinsed with a total of 1.5 ml of saline. The lavage is stored on ice, and the inflammatory cells (leukocytes, neutrophiles and monocytes) are quantified on a CellDyn 3700.

B-13. Analysis of db/db mice

Leptin receptor-deficient db/db mice (Jackson Laboratory) serve as murine model of type 2 diabetes. These animals have, firstly, contractile defects of the heart and, secondly, also renal dysfunction [Belke et al., in Animal Models in Diabetes Research, Methods in Molecular Biology, Vol. 933 (2012); Sayyed et al., Kidney Int 2011, 80, 68-78; Li et al., Acta Pharmacol. Sin. 2010, 31, 560-569]. Male db/db mice with or without unilateral nephrectomy are treated with test substances, and the effect on heart and kidney function is examined.

### B-14. Analysis in the Renal Ischaemia Reperfusion Model (Mouse and Rat)

Experimental data confirm a reduction of the reperfusion damage after renal ischaemia/reperfusion in CCR2-knock out animals [Furuichi et al., J. Am. Soc. Nephrol. 2003, 14, 2503-2515]. In this model, mice or rats are treated with test substances and the effect on kidney function is examined.

### B-15. Analysis in the UUO Model (Mouse and Rat)

Experimental data confirm reduced fibrosis in the unilateral ureteral obstruction (UUO) model in CCR2-knock out animals [Kitagawa et al., Am. J. Pathol. 2004, 165 (1), 237-246]. In this model, mice or rats are treated with test substances and the effect on kidney function is examined.

### B-16. Streptozotocin-Induced Diabetes (Mouse and Rat)

Experimental data confirm reduced kidney damage in the streptozotocin-(STZ)-induced type 1 diabetes model in CCR2-knock out animals or animals that were treated with a CCR2 antagonist [Awad et al., Am. J. Physiol. Renal Physiol. 2011, 301 (6), F1358-F1366; Novikova et al., J. Diabetes Res. 2013, online, Article-ID 965832; WO 2012/041817-A1, pages 87-88]. In this model, mice or rats are treated with test substances and the effect on kidney function is examined.

### B-17. Alport Mouse Model

The effect of test substances can also be demonstrated in the Alport mouse model of kidney damage [Clauss et al., J. Pathol. 2009, 218 (1), 40-47].

### B-18. MCP-1-Induced Monocyte Recruitment in the Rat

Male Sprague Dawley rats (200-250 g; Charles River) are anaesthetized with 5% isoflurane in an anaesthesia cage. In the tolerance stage, MCP-1 (10 μg in 200 μl of NaCl solution) is administered via the tail vein, thus inducing the recruitment of monocytes from bone marrow. 60 minutes after the administration of MCP-1, the rats are re-anaesthetized and sacrificed painlessly, and the blood count (neutrophiles, monocytes) is determined (Advia 2120i, Siemens). The effect of test substances on the MCP-1-induced increase of monocytes measured in the blood is examined.

### C. WORKING EXAMPLES FOR PHARMACEUTICAL COMPOSITIONS

The compounds according to the invention can be converted to pharmaceutical formulations as follows:

### Tablet:

### Composition:

100 mg of the compound according to the invention, 50 mg of lactose (monohydrate), 50 mg of corn starch (native), 10 mg of polyvinylpyrrolidone (PVP 25) (BASF, Ludwigshafen, Germany) and 2 mg of magnesium stearate.

Tablet weight 212 mg, diameter 8 mm, radius of curvature 12 mm

### Production:

The mixture of inventive compound, lactose and starch is granulated with a 5% solution (w/w) of the PVP in water. The granules are dried and mixed with the magnesium stearate for 5 minutes. This mixture is compressed in a conventional tablet press (see above for format of the tablet). The guide value used for the pressing is a pressing force of 15 kN.

Suspension which can be Administered Orally:

### Composition:

1000 mg of the compound according to the invention, 1000 mg of ethanol (96%), 400 mg of Rhodigel® (xanthan gum from FMC, Pennsylvania, USA) and 99 g of water.

A single dose of 100 mg of the inventive compound corresponds to 10 ml of oral suspension.

### Production:

The Rhodigel is suspended in ethanol; the inventive compound is added to the suspension. The water is added while stirring. The mixture is stirred for about 6 h before swelling of the Rhodigel is complete.

### Solution for Oral Administration:

### Composition:

500 mg of the inventive compound, 2.5 g of polysorbate and 97 g of polyethylene glycol 400. A single dose of 100 mg of the inventive compound corresponds to 20 g of oral solution.

### Production:

The inventive compound is suspended in the mixture of polyethylene glycol and polysorbate while stirring. The stirring operation is continued until dissolution of the inventive compound is complete.

i.v. Solution:

The inventive compound is dissolved in a concentration below the saturation solubility in a physiologically acceptable solvent (e.g. isotonic saline, 5% glucose solution and/or 30% PEG 400 solution). The solution is subjected to sterile filtration and dispensed into sterile and pyrogen-free injection vessels.

1. A compound of the formula (I) 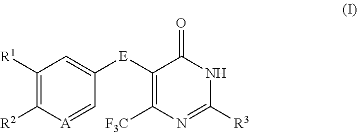
in which
A represents C—H, C—F or N,
E represents CH₂, CH(CH₃), 0, S, S(═O) or S(═O)₂,
R¹ and R², independent of one another represent hydrogen, fluorine, chlorine, methyl, trifluoromethyl or trifluoromethoxy,
where at least one of the two radicals R¹ and R² represents fluorine, chlorine, trifluoromethyl or trifluoromethoxy, and
R³ represents (C₁-C₄)-alkyl which may be substituted by hydroxy, represents cyclopropyl or cyclobutyl or represents a group of the formula —NR^{4A}R^{4B}, —NH—C(═O)—R⁵, —NH—C(═O)—NH₂ or —CH₂—C(═O)—NH₂ in which R^{4A}, R^{4B} and R⁵, independent of one another, represent hydrogen or (C₁-C₄)-alkyl, and their salts, solvates and solvates of the salts.

2. The compound of the formula (I) according to claim 1 in which
A represents C—H, C—F or N,
E represents CH₂, O or S,
R¹ and R², independent of one another, represent hydrogen, fluorine, chlorine, methyl or trifluoromethyl, where at least one of the two radicals R¹ and R² represents fluorine, chlorine or trifluoromethyl, and
R³ represents (C₁-C₄)-alkyl, which may be substituted by hydroxy, represents cyclopropyl or cyclobutyl or represents a group of the formula —NR^{4A}R^{4B}, —NH—C(═O)—R⁵, —NH—C(═O)—NH₂ or —CH₂—C(═O)—NH₂ in which R^{4A}, R^{4B} and R⁵, independent of one another, represent hydrogen or (C₁-C₄)-alkyl, and their salts, solvates and solvates of the salts.

3. The compound of the formula (I) according to claim claim 1 in which
A represents C—H or C—F,
E represents CH₂, O or S,
R¹ represents fluorine, chlorine or trifluoromethyl,
R² represents hydrogen, fluorine, chlorine, methyl or trifluoromethyl and
R³ represents (C₁-C₄)-alkyl which may be substituted by hydroxy, represents cyclopropyl or represents a group of the formula —NR^{4A}R^{4B} or —CH₂—C(═O)—NH₂ in which R^{4A} and R^{4B} independently of one another represent hydrogen, methyl or ethyl, and their salts, solvates and solvates of the salts.

4. The compound of the formula (I) according to claim claim 1 in which
A represents C—H,
E represents CH₂ or O,
R¹ represents fluorine, chlorine or trifluoromethyl,
R² represents fluorine or chlorine and
R³ represents methyl, hydroxymethyl, ethyl, n-propyl, cyclopropyl or a group of the formula —NR^{4A}R^{4B} or —CH₂—C(═O)—NH₂ in which R^{4A} and R^{4B} both represent hydrogen, and their salts, solvates and solvates of the salts.

5. A process for preparing a compound of the formula (I) as defined in claim 1, characterized in that
[A] a compound of the formula (II) 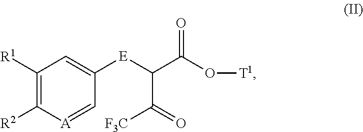
in which A, R¹ and R² have the meanings given in claim 1,
E¹ represents CH₂ or O and
T¹ represents methyl, ethyl, n-propyl or n-butyl, is condensed with a compound of the formula (III) 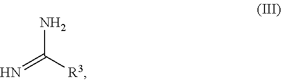
in which R³ has the meaning given in claim 1, or a salt thereof to give a compound of the formula (I-A) 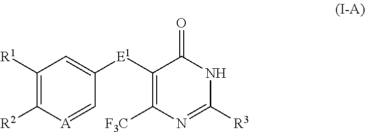
in which A, E¹, R¹, R² and R³ have the meanings given above, or
[B] a compound of the formula (IV) 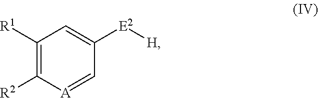
in which A, R¹ and R² have the meanings given in claim 1, and
E² represents O or S, is reacted in the form of an alkali metal salt or in the presence of a base with a compound of the formula (V) 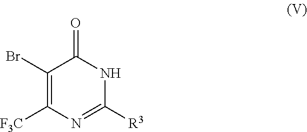
in which R³ has the meaning given in claim 1 to give a compound of the formula (I-B) 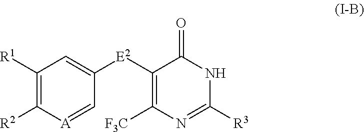
in which A, E², R¹, R² and R³ have the meanings given above, and the resulting compounds of the formulae (I-A) and (I-B) are optionally converted with the appropriate (i) solvents and/or (ii) acids into their solvates, salts and/or solvates of the salts.

6. The compound as defined in claim 1 for treatment and/or prevention of diseases.

7. The compound as defined in claim 1 for use in a method for the treatment and/or prevention of acute coronary syndrome, myocardial infarction, acute and chronic heart failure, acute and chronic kidney failure and acute lung damage.

8. Use of a compound as defined in claim 1 for preparing a medicament for the treatment and/or prevention of acute coronary syndrome, myocardial infarction, acute and chronic heart failure, acute and chronic kidney failure and acute lung damage.

9. A medicament comprising a compound as defined in claim 1 in combination with one or more inert, nontoxic, pharmaceutically suitable excipients.

10. A medicament comprising a compound as defined in claim 1 in combination with one or more further active compounds selected from the group of the antihyperglycaemic agents (antidiabetics), the hypotensive agents, the platelet aggregation inhibitors, the anticoagulants and the HMG-CoA reductase inhibitors (statins).

11. A medicament according to claim 9 for the treatment and/or prevention of acute coronary syndrome, myocardial infarction, acute and chronic heart failure, acute and chronic kidney failure and acute lung damage.

12. A method for treatment and/or prevention of acute coronary syndrome, myocardial infarction, acute and chronic heart failure, acute and chronic kidney failure and acute lung damage in humans and animals by administration of an effective amount of at least one compound as defined in any of claim 1.

13. A method for treatment and/or prevention of acute coronary syndrome, myocardial infarction, acute and chronic heart failure, acute and chronic kidney failure and acute lung damage in humans and animals by administration of an effective amount of a medicament as defined in claim 9.
